(19) United States
(12) Patent Application Publication
(10) Pub. No.: US 2023/0174612 A1
(43) Pub. Date: Jun. 8, 2023

(54) NOTCH RECEPTORS WITH ZINC FINGER-CONTAINING TRANSCRIPTIONAL EFFECTOR

(71) Applicants: The Regents of the University of California, Oakland, CA (US); Trustees of Boston University, Boston, MA (US)

(72) Inventors: Kole T. ROYBAL, San Francisco, CA (US); Iowis ZHU, San Francisco, CA (US); Raymond LIU, San Francisco, CA (US); Ahmad S. KHALIL, Boston, MA (US); Divya ISRANI, Boston, MA (US)

(21) Appl. No.: 17/995,765

(22) PCT Filed: Mar. 24, 2021
(86) PCT No.: PCT/US21/23911

§ 371 Date: Oct. 7, 2022

Related U.S. Application Data
(63) Continuation of application No. PCT/US2020/052244, filed on Sep. 23, 2020.
(60) Provisional application No. 63/007,795, filed on Apr. 9, 2020.
(60) Provisional application No. 63/007,795, filed on Apr. 9, 2020.

(51) Int. Cl. C07K 14/705; C07K 16/28; A61K 35/17; C12N 15/86

(52) U.S. Cl. CPC C07K 14/705; C07K 14/70517; C07K 16/2803; A61K 35/17; C12N 15/86; C07K 2319/81; C07K 2317/622; C07K 2319/03; C07K 2319/035; C07K 2317/24; C07K 2319/715; C12N 2740/15043

(57) ABSTRACT

The present disclosure generally relates to, inter alia, a new class of chimeric Notch receptors containing a synthetic zinc finger transcriptional effector (synZTE) module, engineered to modulate gene expression and cellular activities in a ligand-dependent manner. The new Notch receptors surprisingly retain the ability to transduce signals in response to ligand binding despite that the Notch extracellular subunit (NEC), which includes the negative regulatory region (NRR) previously believed to be essential for the functioning of Notch receptors, is partly or completely deleted. In addition, the synZTE is designed to bind orthogonal DNA target sequences in target organisms which in turn facilitates precise regulation of therapeutic gene expression with minimal off-target activity. Also provided are compositions and methods useful for producing such receptors, nucleic acids encoding same, host cells genetically modified with the nucleic acids, as well as methods for modulating an activity of a cell and/or for the treatment of various health conditions such as cancers.

### CROSS-REFERENCE TO RELATED APPLICATION

The present application claims priority to U.S. Provisional Patent Application Ser. No. 63/007,795, filed on Apr. 9, 2020; and International Application No. PCT/US2020/052244, filed on Sep. 23, 2020. The content of the above-referenced application is herein expressly incorporated by reference in its entirety, including any drawings.

### STATEMENT REGARDING FEDERALLY SPONSORED R&D

This invention was made with government support under grant no. OD025751 awarded by The National Institutes of Health and grant no. HR0011-623666-00 awarded by The Defense Advanced Research Projects Agency. The government has certain rights to the invention.

### INCORPORATION OF THE SEQUENCE LISTING

This application contains a Sequence Listing, which is hereby incorporated by reference in its entirety. The accompanying Sequence Listing text file, named “048536_675002WO_Sequence_Listing_ST25.txt,” was created on Mar. 14, 2021, and is 689 KB.

### FIELD

The present disclosure relates generally to new synthetic cellular receptors that bind cell-surface ligands and having selectable specificities and activities. The disclosure also provides compositions and methods useful for producing such receptors, nucleic acids encoding same, host cells genetically modified with the nucleic acids, as well as methods for modulating gene expression, modulating an activity of a cell, and/or for the treatment of various health conditions or diseases, such as cancer.

### BACKGROUND

The use of targeted cell therapy with precise regulation of therapeutic gene expression is a core strategy for the treatment of many diseases or health disorders. For example, recent advances in synthetic biology are enabling the development of new gene therapies and engineered-cell therapies. However, an important problem limiting the development of engineered cell therapies in humans is the regulation of therapeutic gene expression to reduce or eliminate interactions causing significant side effects on administration of an engineered therapeutic cell such as, off-target activity, on-target, off-tumor activity, and difficulties in modulating or turning off the therapeutic engineered cell's activity when needed. A possible solution to these problems is to modulate therapeutic gene expression and/or cellular behavior in a precise manner through the development and delivery of synthetic therapeutic systems, for example, using synthetic cellular receptors capable of binding cell-surface ligands, and capable of targeting responsive elements to conditionally induce or silence therapeutic gene expression, and/or modulate an activity of a target cell.

Examples of some first-generation synthetic therapeutic systems include synthetic derivatives of Notch receptors, which are often referred to as “SynNotch receptors” and contain structural modifications of the core force-sensing module of wild-type Notch receptors to regulate customizable intracellular trans-activators with user-defined ligand binding domains by replacing the extracellular ligand-binding domain, which in wild-type Notch contains multiple EGF-like repeats, with an antibody derivative, and replacing the cytoplasmic domain with a transcription activator of choice, while still relying on the functionality of the Notch NRR (K T Roybal et al., Cell 2016 Oct. 6; 167(2):419-32) and L. Morsut et al., Cell (2016) 164:780-91). Generally, the signaling of these first-generation SynNotch correlates with ligand binding, but it is often difficult to adjust the sensitivity and response of the receptor. Additionally, these engineered proteins are large and approach the packaging limits of traditional lentiviral delivery schemes, preventing efficient delivery and expression, and the addition of other useful molecular components. For example, the Notch regulatory regions (NRRs), previously believed to be essential for the functioning of Notch and SynNotch receptors, spans approximately 160 amino acids, making this domain alone the size of some mature proteins such as insulin or epidermal growth factor (EGF). This is believed to cause expression of the first-generation SynNotch receptors less efficient and, due to vector capacity-related size constraints, the resulting SynNotch receptors can exceed the capacity of some cloning and transfection vectors.

The disclosures provided herein address these shortcomings and provide additional benefits.

### SUMMARY

The present disclosure relates generally to a new class of chimeric Notch receptors containing a synthetic zinc finger transcriptional effector (synZTE) module, engineered to modulate gene expression and cellular activities in a ligand-dependent manner. The activity of these synZTE-containing Notch receptors can be controlled by the presence of an extracellular ligand, allowing for spatial and temporal control of specific gene expression in mammalian cells, as well as for use in modulating cell activities or in treating various health conditions or diseases. Particularly, provided herein are synZTE-containing Notch receptors that, surprisingly, retain the ability to transduce signals in response to ligand binding despite that the Notch extracellular subunit (NEC), which includes the negative regulatory region (NRR), is partly or completely removed. Additionally, these new synZTE-containing Notch receptors are functional, whereas SynNotch receptors fail to exhibit a detectable signal. In some embodiments, these new receptors incorporate a synthetic DNA-binding zinc finger protein domain (“synZF protein domain”) that is designed to bind orthogonal DNA target sequences, and have little or no binding to existing DNA sequences in organisms, which in turn allows precise regulation of therapeutic gene expression with minimal off-target activity. In some embodiments described below, the synZF-containing protein domain is operably linked to an effector domain through which the engineered Notch receptor exerts it effect. As described in greater detail below, the effector domain can be a transcriptional effector domain such as, for example, a transcription activating domain, a transcription repressor domain, or an epigenetic effector domain. Without being bound to any particular theory, it is believed that by partly or completely removing the native Notch NEC and NRR, this design of the synZTE-containing Notch receptors disclosed herein allows for nucleic acids encoding the receptors to be made smaller than existing first-generation SynNotch-encoding polynucleotides, which in turn facilitates the use of viral vectors having more limited capacity, and/or facilitates the inclusion of additional elements that would otherwise be excluded by vector capacity-related size constraints.

In one aspect, provided herein are chimeric polypeptides including, from N-terminus to C-terminus: (a) an extracellular ligand-binding domain having a binding affinity for a selected ligand; (b) a linking polypeptide having: (i) at least about 80%, 85%, 90%, 95%, 96%, 97%, 98%, 99%, or 100% sequence identity to a Notch juxtamembrane domain (JMD) wherein a LIN-12-Notch repeat (LNR) and/or a heterodimerization domain (HD) of a Notch receptor has been deleted; (ii) at least about 80%, 85%, 90%, 95%, 96%, 97%, 98%, 99%, or 100% sequence identity to a polypeptide hinge domain; or (iii) a sequence of about 2 to about 40 amino acid residues; (c) a transmembrane domain having at least about 80%, 85%, 90%, 95%, 96%, 97%, 98%, 99%, or 100% sequence identity to the transmembrane domain of a Type 1 transmembrane receptor and including one or more ligand-inducible proteolytic cleavage sites; and (d) an intracellular domain including a zinc finger-containing transcriptional effector (ZTE), wherein binding of the selected ligand to the extracellular binding domain induces cleavage at a ligand-inducible proteolytic cleavage site within the transmembrane domain.

Non-limiting exemplary embodiments of the chimeric polypeptides provided herein include one or more of the following features. In some embodiments, the chimeric polypeptide further includes a stop-transfer-sequence (STS) in between the transmembrane domain and the intracellular domain. In some embodiments, the STS is operably linked between the transmembrane domain and the intracellular domain. In some embodiments, the linking polypeptide includes an amino acid sequence having at least about 80%, 85%, 90%, 95%, 96%, 97%, 98%, 99%, or 100% sequence identity to a Notch JMD according to any one of SEQ ID NOS: 11-19. In some embodiments, the linking polypeptide has a length ranging from 1 to 40 amino acid residues. In some embodiments, the linking polypeptide includes a glycine-serine linker. In some embodiments, the linking polypeptide has the amino acid sequence (GGS)n, wherein n is an integer from 1 to about 50. In some embodiments, n is 18, 15, 12, 9, 6, or 3. In some embodiments, n is 3. In some embodiments, the linking polypeptide includes an amino acid sequence at least about 80%, 85%, 90%, 95%, 96%, 97%, 98%, 99%, or 100% sequence identity to any one of SEQ ID NOS: 25-28.

In some embodiments, the linking polypeptide of the chimeric polypeptides disclosed herein includes a hinge domain capable of promoting oligomer formation of the chimeric polypeptide via intermolecular disulfide bonding. In some embodiments, the hinge domain is derived from a CD8α hinge domain, a CD28 hinge domain, a PD-1 hinge domain, a CTLA4 hinge domain, an OX40 hinge domain, an IgG1 hinge domain, an IgG2 hinge domain, an IgG3 hinge domain, and an IgG4 hinge domain, or a functional variant of any thereof. In some embodiments, the hinge domain is derived from a CD8α hinge domain or a functional variant thereof. In some embodiments, the hinge domain is derived from a CD28 hinge domain or a functional variant thereof. In some embodiments, the hinge domain is derived from an OX40 hinge domain or a functional variant thereof. In some embodiments, the hinge domain is derived from an IgG4 hinge domain or a functional variant thereof. In some embodiments, the hinge domain includes an amino acid sequence having at least about 80%, 85%, 90%, 95%, 96%, 97%, 98%, 99%, or 100% sequence identity to any one of SEQ ID NOS: 20-24.

In some embodiments, the stop-transfer-sequence (STS) between the transmembrane domain and the intracellular domain includes an amino acid sequence having at least about 80%, 85%, 90%, 95%, 96%, 97%, 98%, 99%, or 100% sequence identity to any one of SEQ ID NOS: 39-54. In some embodiments, the transmembrane domain includes an amino acid sequence having at least 80%, 85%, 90%, 95%, 96%, 97%, 98%, 99%, or 100% sequence identity to any one of SEQ ID NOS: 29-38.

In some embodiments, the ZTE of the chimeric polypeptide disclosed herein includes: (a) a first domain including a DNA-binding zinc finger protein domain (ZF protein domain), and (b) a second domain through which the ZTE exerts its effect (effector domain), wherein the ZTE has a structure according to Formula I:

[effector domain]ₐ-[ZF protein domain]-[effector domain]_{b} (Formula I),

wherein a and b are each independently an integer from 0 to about 5, and at least one of a and b is not 0; wherein the ZF protein domain includes 1 to about 10 zinc finger arrays (ZFA); wherein the ZFA includes about 6 to about 8 zinc finger motifs having the Formula II (from N-terminal to C-terminal):

X_{c}CX_{d}CXₑ-(helix)-HX_{f}H-L² (Formula II),

wherein L² is a linker peptide having about 4-6 amino acid residues, C is Cys, H is His, each X is independently any amino acid, c is an integer from 0 to 3, d is an integer from 1 to 5, e is an integer from 2 to 7, f is an integer from 3 to 6, and (helix) is a peptide domain of about 6 amino acids that forms an α-helix, wherein the ZFA is capable of binding a specific nucleic acid sequence.

In some embodiments, the ZFA of the ZTE is capable of specifically binding to a target nucleic acid sequence selected from the group consisting of SEQ ID NOs: 61-71. In some embodiments, the ZFA includes a sequence having at least about 90% identity to a sequence selected from the group consisting of SEQ ID NOs: 55-60. In some embodiments, the ZFA has a sequence having about 100% identity to a sequence selected from the group consisting of SEQ ID NOs: 55-60.

In some embodiments, the effector domain of the ZTE includes an effector domain selected from the group consisting of a transcription activating domain, a transcription repressor domain, or an epigenetic effector domain. In some embodiments, the effector domain includes a transcription activating domain selected from the group consisting of Herpes Simplex Virus Protein 16 (HSV VP16) activation domain; an activation domain consisting of four tandem copies of VP16 (VP64); a p65 activation domain of NFκB; an Epstein-Barr virus R transactivator activation domain (Rta); a tripartite activator consisting of VP64, p65, and Rta activation domains (VPR); and a histone acetyltransferase core domain of the human E1A-associated protein p300 (p300 HAT core activation domain). In some embodiments, the effector domain includes a transcription repressor domain selected from the group consisting of a Kruppel associated box repression domain (KRAB); a Repressor Element Silencing Transcription Factor repression domain (REST); a WRPW motif of the hairy-related basic helix-loop-helix repressor proteins repression domain (WRPW); a DNA (cytosine-5)-methyltransferase 3B repression domain (DNMT3B); and an HP1 alpha chromoshadow repression domain. In some embodiments, the effector domain includes an epigenetic effector domain selected from the group consisting of a DNA methyltransferase DNMT (DNMT1, DNMT3), HAT1, GCN5, PCAF, MLL, SET, DOT1, SUV39H, G9a, KAT2A/B, EZH1/2, TET1/2, a SIRT family protein effector domain, LSD1, and a KDM family protein effector domain. In some embodiments, the effector domain includes a domain from a human protein. In some embodiments, the intracellular domain further includes a nuclear transport signal sequence.

In another aspect, provided herein are recombinant nucleic acids including a nucleotide sequence that encodes a chimeric polypeptide as disclosed herein. In some embodiments, the nucleotide sequence is incorporated into an expression cassette or an expression vector. In some embodiments, the expression vector is a viral vector. In some embodiments, the viral vector is a lentiviral vector, an adenovirus vector, an adeno-associated virus vector, or a retroviral vector.

In some embodiments, the recombinant nucleic acid further includes a response element, wherein the response element includes: (a) a ZFA target sequence; (b) an engineered responsive promoter operably linked to the ZF target sequence; and (c) a polynucleotide of interest. In some embodiments, the polynucleotide of interest encodes a regulatory RNA, a regulatory protein, a therapeutic protein, or a detectable label. In some embodiments, the detectable label is a fluorescent protein. In some embodiments, the therapeutic protein is a chimeric antigen receptor (CAR). In some embodiments, the regulatory RNA is an siRNA, shRNA, or miRNA.

In another aspect, provided herein are recombinant cells including (a) a chimeric polypeptide as disclosed herein and/or (b) a recombinant nucleic acid as disclosed herein. Also provided, in a related aspect, are cell cultures including at least one recombinant cell as disclosed herein and a culture medium. In some embodiments, the recombinant cell is a eukaryotic cell. In some embodiments, the eukaryotic cell is a mammalian cell. In some embodiments, the mammalian cell is an immune cell, a neuron, an epithelial cell, and endothelial cell, or a stem cell. In some embodiments, the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell.

In some embodiments, the recombinant cell further includes an engineered response element including i) a ZFA target sequence to which a ZFA of the ZTE of the chimeric polypeptide specifically binds, ii) a promoter sequence, wherein the nucleic acid target sequence is operably linked to the 5′ end of the promoter sequence, and iii) a polynucleotide of interest operably linked to the promoter sequence, wherein binding of the ZTE to the ZFA target sequence modulates transcription initiation of a polynucleotide of interest. In some embodiments, the engineered response element is present in a nucleic acid vector, plasmid, DNA minicircle, minichromosome, or chromosome. In some embodiments, the polynucleotide of interest encodes a protein, regulatory RNA, or an antisense oligonucleotide. In some embodiments, the ZFA target sequence includes a sequence that is orthogonal to the recombinant cell genome. In some embodiments, the ZFA target sequence includes a nucleotide sequence selected from the group consisting of SEQ ID NOs: 61-71.

Another aspect relates to methods for making an engineered cells that include: (a) providing a cell capable of protein expression; and (b) transducing the cell with a recombinant nucleic acid as disclosed herein. In some embodiments, the method further includes (c) transducing the cell with a recombinant nucleic acid that encodes a response element, wherein the response element includes: (i) a ZFA target sequence; (ii) an engineered responsive promoter operably linked to the ZF target sequence; and (iii) a polynucleotide of interest.

In another aspect, provided herein are pharmaceutical compositions including a pharmaceutical acceptable carrier and one or more of the following: (a) a recombinant nucleic acid as disclosed herein, and (b) a recombinant cell as disclosed herein. In some embodiments, the disclosed pharmaceutical composition includes a recombinant nucleic acid as disclosed herein and a pharmaceutically acceptable carrier. In some embodiments, the recombinant nucleic acid is encapsulated in a viral capsid or a lipid nanoparticle.

In another aspect, provided herein are methods for modulating an activity of a target cell in an individual, including administering to the individual an effective number of the recombinant cells as disclosed herein, wherein the recombinant cells modulate an activity of the target cell in the individual.

Another aspect relates to methods for modulating an activity of a cell, including: (a) providing a recombinant cell as disclosed herein; and (b) contacting the recombinant cell with the selected ligand, wherein binding of the selected ligand to the extracellular ligand-binding domain results in cleavage of a ligand-inducible proteolytic cleavage site and release of the intracellular domain, wherein the release of the intracellular domain results in modulation of an activity of the recombinant cell. In some embodiments, the release of the intracellular domain results in binding of the ZTE of the released intracellular domain to a ZFA target sequence, which results in modulation of the expression initiation of a polynucleotide of interest, which results in modulation of an activity of the recombinant cell. In some embodiments, the activity of the cell to be modulated is selected from the group consisting of: expression of a selected gene, proliferation, apoptosis, non-apoptotic death, differentiation, dedifferentiation, migration, secretion of a molecule, cellular adhesion, and cytolytic activity. In some embodiments, the ZTE modulates expression of a gene. In some embodiments, the ZTE modulates expression of a heterologous gene product. In some embodiments, the gene product is selected from the group consisting of chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, an RNA guided nuclease, a site-specific nuclease, a T cell receptor, a toxin, a toxin derived protein, a transcriptional regulator, a transcriptional activator, a transcriptional repressor, a translational regulator, a translational activator, a translational repressor, an activating immuno-receptor, an antibody, an apoptosis inhibitor, an apoptosis inducer, an engineered T cell receptor, an immuno-activator, an immuno-inhibitor, an immune cell receptor, and an inhibiting immuno-receptor. In some embodiments, the released ZTE modulates differentiation of the cell, and wherein the cell is an immune cell, a stem cell, a progenitor cell, or a precursor cell.

Another aspect relates to methods for treating a health condition, (e.g., disease) in an individual, including administering to the individual an effective number of the recombinant cells of the disclosure, wherein the recombinant cells treat the health condition in the individual.

In another aspect, some embodiments of the disclosure relate to kits for modulating an activity of a cell, inhibiting a target cancer cell, or treating a health condition (e.g., disease) in an individual in need thereof, wherein the systems include one or more of: a chimeric polypeptide of the disclosure; a nucleic acid of the disclosure; a recombinant cell of the disclosure; and/or a pharmaceutical composition of the disclosure. In a related aspect, some embodiments of the disclosure relate to kits for modulating an activity of a cell, wherein the kits include: (a) a chimeric polypeptide of the disclosure; (b) a nucleic acid of the disclosure; and (c) and an engineered response element including: (i) a ZFA target sequence; (ii) an engineered responsive promoter operably linked to the ZFA target sequence; and (iii) a polynucleotide of interest; wherein binding of the ZTE to the nucleic acid target sequence modulates transcription initiation of the polynucleotide of interest.

Yet another aspect of the disclosure is the use of one or more of: a chimeric polypeptide of the disclosure; a polynucleotide of the disclosure; a recombinant cell of the disclosure; and a pharmaceutical composition of the disclosure; for the treatment of a health condition, such as a disease. In some embodiments, the disease is cancer.

Another aspect of the disclosure is the use of one or more of: a chimeric polypeptide of the disclosure; a polynucleotide of the disclosure; a recombinant cell of the disclosure; or a pharmaceutical composition of the disclosure; for the manufacture of a medicament for the treatment of a health condition.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 schematically illustrates non-limiting examples of engineered Notch receptor variants. SynNotch variants leverage the core force-sensing module of Notch (ligand-inducible cleavage at the S3 site) to regulate customizable intracellular transactivators with user-defined ligand binding domains. In MiniNotch variants, much of the regulatory region is further truncated. HingeNotch variants additionally feature disulfide-mediated oligomerization due to the insertion of a Hinge domain (for instance, a hinge domain from CD8).

FIG. 2 schematically summarizes the results from experiments performed to assess functionality of three exemplary engineered Notch receptors variants coupled with zinc finger-based transcriptional effectors (synZTE).

FIG. 3 schematically summarizes the results of experiments performed to further illustrate incapability of two exemplary human SynNotch receptor derivatives in accordance with some embodiments of the disclosure, which contained either zinc-finger transcriptional effector ZF3 or ZF10. In these experiments, Jurkat T-cells were transduced with anti-CD19 SynNotch receptors containing either ZF3 or ZF10 transcriptional effectors with unique DNA binding specificities, along with their cognate mCitrine reporter. Reporter gene expression data indicates receptor-mediated activation with antigen-negative cells (+K562) vs. antigen-positive K562 cells (+K562 CD19) after 24 hours of co-incubation. The results demonstrate that the synNotch receptors failed to activate.

FIG. 4 summarizes the results of experiments performed to assess functionality of Hinge-Notch receptors in accordance with some embodiments of the disclosure, each having one of six exemplary zinc-finger transcriptional effectors: ZF2, ZF3, ZF4, ZF6, ZF10, and ZF11. In these experiments, primary CD4+ T-cells from two different donors were transduced with anti-CD19 HingeNoch receptors containing one of six different SynTF transactivators with unique DNA binding specificities, along with their cognate BFP-expressing reporters. Reporter expression data indicates receptor activation with antigen-negative cells (+K562) vs. antigen-positive K562 cells (+K562 CD19) after 48 hours of co-incubation (N=2; error bars represent standard deviation).

FIG. 5 schematically summarizes the normalized fluorescence activation profiles of the T-cells described in FIG. 4 co-incubated with antigen-negative (crossed) or antigen-positive (slashed) K562 target cells.

FIG. 6 schematically illustrates expression levels of six exemplary HingeNotch-zinc-finger synTF receptors described in FIGS. 4 and 5. In FIG. 6, expression levels of HingeNotch-zinc-finger synTF receptors are indicated on vertical axis and the cognate reporter is indicated on horizontal axis.

FIGS. 7A-7C schematically summarize the results of experiments performed to optimize the functionality of synZTE-containing HingeNotch receptors. FIG. 7A shows a sequence schematic of loci within a lentiviral expression construct for an exemplary synZTE-containing HingeNotch ZF6, i.e., pDP1160 (SEQ ID NO: 7), that were interspersed with functionally unannotated sequences. These include (i) an alanine between the HingeNotch core functional region and the nuclear localization sequence (NLS) of the synZTE-containing HingeNotch (Linker 1), (ii) several potentially non-essential regions between the NLS and zinc-finger domain consisting of a polypeptide (Linker 2), (iii) the expression product of an XhoI restriction enzyme site (Linker 3), (iv) a flexible linker glycine-serine (Linker 4), (v) the expression product KpnI and NheI restriction enzyme sites (Linker 5), and also (vi) the expression product of BamHI and SbfI site restriction enzyme sites between the zinc finger and transactivation domain p65 (Linker 6)). Additionally, the 108 bp between the p65 transactivation domain and the WPRE were replaced with an 8 bp NotI site (Linker 7). Moreover, in one construct, the full-length transactivation domain p65 was replaced with a minimal sequence starting at residue P68. FIG. 7B summarizes BFP expression from Jurkat cells transduced with a ZF6BD-BFP reporter construct and a panel of anti-CD19 HingeNotch-ZF6 expression vectors bearing the indicated linker deletions or modifications. In these experiments, cells were stimulated with unmodified K562 cells (left panel) or CD19-expression K562 cells (right panel). FIG. 7C depicts percentage of BFP-expressing Jurkat cells (left panel) and BFP MFI (right panel) tabulated for the data presented in FIG. 7B.

FIGS. 8A-8C pictorially summarize of the expression profiles of the original synZTE-containing HingeNotch receptor versus partially minimized synZTE-HingeNotch variants bearing ZF6 or ZF10, as described in FIGS. 7A-7C above. As shown in FIG. 8A, the minimized versions that bear none of the linker sequences deleted but that retained the full-length transactivation domain p65 express equally or better than their counterparts with all linkers intact. FIG. 8B shows BFP expression from the construct referenced in FIG. 8A after stimulation with unmodified or CD19-expressing K562 cells. FIG. 8C shows percent BFP-expressing T-cells (left) and BFP MFI (right) tabulated for the data in FIG. 8B.

FIGS. 9A-9B schematically summarize the results of experiments performed modifying nuclear localization sequence (NLS) to modulate receptor activity. FIG. 9A shows BFP expression of primary CD4+ T-cells transduced with MiniNotch receptor variants bearing synthetic zinc finger-containing transcriptional activators (SynTFs) consisting of the ZF3 zinc finger and transactivation domain p65, with either the original SV40 NLS or the hNotch1 NLS. FIG. 9B shows BFP expression MFI quantified for the experiment shown in FIG. 9A.

FIG. 10 schematically summarizes the results of experiments performed to evaluate the activation performance of synZTE-containing HingeNotch receptors relative to Gal4-VP64.

FIG. 11 schematically summarizes the results of experiments performed to illustrate the specific target cell killing via synZTE-containing HingeNotch receptors.

### DETAILED DESCRIPTION OF THE DISCLOSURE

The present disclosure generally relates to, among other things, a new class of chimeric Notch receptors that include a synthetic zinc finger-containing transcriptional effector (synZTE) module and are engineered to modulate transcriptional regulation in a ligand-dependent manner. Particularly, the new receptors (termed “synZTE-containing Notch receptors”) surprisingly retain the ability to transduce signals in response to ligand binding despite that the Notch extracellular subunit (NEC), which includes the negative regulatory region (NRR) previously believed to be essential for the functioning of Notch receptors, is partly or completely removed. As described below, the new class of chimeric Notch receptors disclosed herein does not occur in nature, and can be engineered, designed, or modified so as to provide desired and/or improved properties, e.g., in modulating transcription. The activity of these synZTE-containing Notch receptors can be controlled by the presence of an extracellular ligand, allowing for spatial and temporal control of specific gene expression in mammalian cells, as well as for use in modulating cell activities or in treating various health conditions (e.g., diseases). The demonstration that the new synZTE-containing Notch receptors as disclosed herein are not only functional but demonstrate enhanced biologic activity is surprising and is completely contrary to the teachings in the field. In some embodiments, the chimeric Notch receptors disclosed herein bind a target cell-surface ligand, which triggers proteolytic cleavage of the chimeric receptor and release of a transcriptional effector (e.g., synZTE) that modulates a custom transcriptional program in the cell.

These chimeric receptors of the disclosure incorporate a synthetic DNA-binding zinc finger protein domain (synSF protein domain) that is designed to bind orthogonal DNA target sequences and has little or no binding activity to existing DNA sequences in organisms, which in turn facilitates precise regulation of therapeutic gene expression with minimal off-target activity.

The combination of a synZTE and a chimeric Notch receptor capable of specifically binding a target cell-surface ligand forms a unique expression system that is artificial, scalable, and regulatable, for the expressions of desired genes and response elements, with no or minimal effects on the expression of endogenous genes, meaning no or minimal off-site gene regulation of endogenous genes. Further, the present disclosure demonstrates that an augmented transactivation domain fusion (e.g., p65-GR1 or p65-HSF1) enables chimeric Notch receptors (e.g., HingeNotch receptors) bearing zinc finger DNA binding domains to activate to a higher output level. In some embodiments, the augmented output level can be similar to that of the original Gal4-VP64-containing receptor.

The disclosure also provides compositions and methods useful for producing such receptors, nucleic acids encoding same, cells genetically modified with the nucleic acids, as well as methods for modulating an activity of a cell and/or for the treatment of various health conditions or diseases such as cancers.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

The terms “administration” and “administering”, as used herein, refer to the delivery of a composition or formulation as disclosed herein by an administration route including, but not limited to, intravenous, intra-arterial, intracranial, intramuscular, intraperitoneal, subcutaneous, intramuscular, or combinations thereof. The term includes, but is not limited to, administration by a medical professional and self-administration.

“Cancer” refers to the presence of cells possessing several characteristics typical of cancer-causing cells, such as uncontrolled proliferation, immortality, metastatic potential, rapid growth and proliferation rate, and certain characteristic morphological features. Some types of cancer cells can aggregate into a mass, such as a tumor, but some cancer cells can exist alone within a subject. A tumor can be a solid tumor, a soft tissue tumor, or a metastatic lesion. As used herein, the term “cancer” also encompasses other types of non-tumor cancers. Non-limiting examples include blood cancers or hematologic malignancies, such as leukemia, lymphoma, and myeloma. Cancer can include premalignant, as well as malignant cancers.

The terms “cell”, “cell culture”, “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the originally cell, cell culture, or cell line.

The term “modulating,” in relation to the expression or activity of a polypeptide refers a change in the expression or activity of the polypeptide. Modulation includes both activation (e.g., increase, induce, stimulate) and repression or inhibition (e.g., decrease, reduce, inhibit), or otherwise affecting the expression or activity of the polypeptide. The term may also refer to decreasing, reducing, inhibiting, increasing, inducing, activating, or otherwise affecting the activity of a gene encoding the polypeptide which can include, but is not limited to, modulating transcriptional activity.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the orthogonal DNA target sequences described herein or the promoter sequence in a nucleic acid construct, or in an engineered response element means that the orthogonal DNA target sequences and the promoters are in-frame and in proper spatial and distance away from a polynucleotide of interest coding for a protein or an RNA to permit the effects of the respective binding by transcription factors or RNA polymerase on transcription.

As used herein, the term “orthogonal DNA sequence elements” refers to those DNA sequences that are not found or are rarely represented in the eukaryotic genome in nature.

As used herein, the term “orthogonus” when use in context with nucleic acid sequences such as DNA refers to those not naturally found in nature.

The term “percent identity” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

As used herein, and unless otherwise specified, a “therapeutically effective amount” of an agent is an amount sufficient to provide a therapeutic benefit in the treatment or management of a disease, e.g., the cancer, or to delay or minimize one or more symptoms associated with the disease. A therapeutically effective amount of a compound means an amount of therapeutic agent, alone or in combination with other therapeutic agents, which provides a therapeutic benefit in the treatment or management of the disease. The term “therapeutically effective amount” can encompass an amount that improves overall therapy of the disease, reduces or avoids symptoms or causes of the disease, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, etc. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Notch Receptors

Notch receptors are transmembrane proteins that mediate cell-cell contact signaling and play a central role in development and other aspects of cell-to-cell communication. Notch receptors have a modular domain organization. The Notch extracellular subunit (NEC) of wild type Notch receptors consist of a series of N-terminal epidermal growth factor receptor (EGFR)-like repeats that are responsible for ligand binding. O-linked glycosylation of these EGFR repeats, including modification by O-fucose, Fringe, and Rumi glycosyltransferases, also modulates the activity of Notch receptors in response to different ligand subtypes in flies and mammals. The EGFR repeats are followed by three LIN-12/Notch repeat (LNR) modules, which are unique to Notch receptors, and are widely reported to participate in preventing premature receptor activation. The heterodimerization (HD) domain of Notch1 is divided by furin cleavage, so that its N-terminal part terminates the Notch extracellular subunit (NEC), and its C-terminal half constitutes the beginning of the Notch transmembrane (NTM) subunit. Following the extracellular HD-C region of the NEC is a transmembrane segment and an intracellular region (ICN), which includes a transcriptional activator.

Notch receptors mediate cell-cell contact signaling and play a central role in development and other aspects of cell-to-cell communication, e.g., communication between two contacting cells, in which one contacting cell is a “receiver” cell and the other contacting cell is a “sender” cell. Notch receptors expressed in a receiver cell recognize their ligands (e.g., the delta/serrate/lag, or “DSL” family of proteins), expressed on a sending cell. The engagement of notch and delta on these contacting cells leads to two-step proteolysis of the notch receptor that ultimately causes the release of the intracellular portion of the receptor from the membrane into the cytoplasm. Notch has a metalloprotease cleavage site (denoted “S2”), which is normally protected from cleavage by the Notch negative regulatory region (NRR), a domain consisting of three LNR modules and an HD of the NEC. It is believed that this two-step proteolysis is regulated by the force exerted by the sending cell: the DSL ligand pulls on the Notch receptor and changes the conformation of the negative regulatory region, exposing the metalloprotease site. That site is then cleaved by a constitutively active protease, releasing the extracellular binding portion and negative regulatory region (NRR) of the receptor. Release of the extracellular binding portion of the receptor in turn exposes another intramembrane cleavage site(s) (denoted “S3”), which is/are cleaved by gamma secretase within the cell membrane to release the nuclear homing intracellular domain from the cell membrane. W. R. Gordon et al., Dev Cell (2015) 33:729-36. This released domain alters receiver cell behavior by functioning as a transcriptional regulator.

Notch receptors are involved in and are required for a variety of cellular functions during development and are important for the function of a vast number of cell-types across species. Evolutionary divergence of vertebrates and invertebrates has been accompanied by at least two rounds of gene duplication involving the Notch receptors: flies possess a single Notch gene, worms two (GLP-1 and LIN-12), and mammals four (NOTCH1-4). Transduction of Notch signals relies on three key events: (i) ligand recognition, (ii) conformational exposure of the ligand-dependent cleavage site, and (iii) assembly of nuclear transcriptional activation complexes.

Canonical Notch signals are transduced by a process called regulated intramembrane proteolysis. Notch receptors are normally maintained in a resting, proteolytically resistant conformation on the cell surface, but ligand binding initiates a proteolytic cascade that releases the intracellular portion of the receptor (also known as intracellular notch (ICN) or Notch intracellular domain (NICD)) from the membrane. The critical, regulated cleavage step is effected by ADAM metalloproteases and occurs at a site called S2 immediately external to the plasma membrane. This truncated receptor, dubbed NEXT (for Notch extracellular truncation), remains membrane tethered until it is processed at site S3 by gamma secretase, a multiprotein enzyme complex.

After gamma secretase-mediated cleavage, the ICN ultimately enters the nucleus, where it assembles a transcriptional activation complex that contains a DNA-binding transcription factor termed CSL (C-promoter-binding factor in mammals; also known as RBP-J)/Suppressor of hairless in Drosophila melanogaster or Lag1 in Caenorhabditis elegans), and a transcriptional coactivator of the Mastermind/Lag-3 family. This complex then engages additional coactivator proteins such as p300 to recruit the basal transcription machinery and activate the expression of downstream target genes.

Additional information regarding Notch receptors and Notch-mediated cell signaling can be found in, for example, W. R. Gordon et al., Dev Cell (2015) 33:729-36 and W. R. Gordon et al., J. Cell Sci. (2008) 121:3109-19, both of which are hereby incorporated by reference.

### Compositions of the Disclosure

As described in greater detail below, one aspect of the present disclosure relates to a new class of chimeric Notch receptors that include a synthetic zinc finger-containing transcriptional effector (synZTE) module and are engineered to modulate transcriptional regulation in a ligand-dependent manner with various advantages over existing first-generation SynNotch receptors. For example, since natural Notch receptors are large with the NEC subunit containing several dozen tandem EGFR-like repeats, by partly or completely omitting the Notch regulatory regions, or even removing the entire NEC subunit, nucleic acids encoding the synZTE-containing Notch receptors of the disclosure can be made smaller than natural Notch receptors and existing SynNotch-encoding polynucleotides, which enables the use of vectors having more limited capacity, or the inclusion of additional elements that would otherwise be excluded by vector capacity-related size constraints.

In addition, these new receptors incorporate a synthetic DNA-binding zinc finger protein domain (synSF protein domain) that is designed to bind orthogonal DNA target sequences, and has little or no binding activity to existing DNA sequences in organisms. The combination of a synZTE and a chimeric Notch receptor capable of specifically binding a target cell-surface ligand forms a unique expression system that is artificial, scalable, and regulatable, for the expressions of desired genes and response elements, with no or minimal effects on the expression of endogenous genes, meaning no or minimal off-site gene regulation of endogenous genes.

### Chimeric Polypeptides

As described in greater detail herein, some embodiments of the present disclosure relate to novel, non-naturally occurring chimeric polypeptides engineered to modulate transcriptional regulation in a ligand-dependent manner. In particular, the new receptors, even though derived from Notch, do not require the Notch regulatory regions (NRRs) previously believed to be essential for the functioning of the receptors. Furthermore, the new engineered receptors described herein incorporate an extracellular oligomerization domain (e.g., hinge domain) to promote oligomerization to form higher order oligomeric, e.g., dimeric or trimeric, forms of the chimeric receptors. In some embodiments, the hinge domain includes polypeptide motifs capable of promoting oligomer formation of the chimeric polypeptide via intermolecular disulfide bonding. The extracellular oligomerization domain can replace part or all of the Notch extracellular domain. In some embodiments, the receptors disclosed herein bind a target cell-surface ligand, which triggers proteolytic cleavage of the receptors and release of a transcriptional regulator that modulates a custom transcriptional program in the cell.

In some embodiments, the chimeric polypeptide of the disclosure includes, from N-terminus to C-terminus: (a) an extracellular ligand-binding domain having a binding affinity for a selected ligand; (b) a linking polypeptide having a sequence of about 2 to about 40 amino acid residues; (c) a transmembrane domain having at least about 80% sequence identity to the transmembrane domain of a Type 1 transmembrane receptor and including one or more ligand-inducible proteolytic cleavage sites; and (d) an intracellular domain including a zinc finger-containing transcriptional effector (ZTE), wherein binding of the selected ligand to the extracellular binding domain induces cleavage at a ligand-inducible proteolytic cleavage site within the transmembrane domain. In some embodiments, the linking polypeptide has at least about 80% sequence identity to a Notch JMD wherein a LIN-12-Notch repeat (LNR) and/or a heterodimerization domain (HD) of a Notch receptor has been deleted. In some embodiments, the linking polypeptide has at least about 80% sequence identity to a hinge domain, e.g., an oligomerization domain containing one or more polypeptide motifs that promote oligomer formation of the chimeric polypeptides via intermolecular disulfide bonding.

Extracellular Ligand-Binding Domains (ECD)

In some embodiments, the ECD of the chimeric polypeptide receptors disclosed herein (e.g., synZTE-containing Notch receptors) has a binding affinity for one or more target ligands. The target ligand is expressed on a cell surface, or is otherwise anchored, immobilized, or restrained so that it can exert a mechanical force on the chimeric receptor. As such, without being bound to any particular theory, binding of the ECD of a chimeric receptor provided herein to a cell-surface ligand does not necessarily remove the target ligand from the target cell surface, but instead enacts a mechanical pulling force on the chimeric receptor. For example, an otherwise soluble ligand may be targeted if it is bound to a surface, or to a molecule in the extracellular matrix. In some embodiments, the target ligand is a cell-surface ligand. Non-limiting examples of suitable ligand types include cell surface receptors, adhesion proteins, carbohydrates, lipids, glycolipids, lipoproteins, and lipopolysaccharides that are surface-bound, integrins, mucins, and lectins. In some embodiments, the ligand is a protein. In some embodiments, the ligand is a carbohydrate.

In some embodiments, the ECD includes the ligand-binding portion of a receptor. In some embodiments, the ECD includes an antigen-binding moiety that binds to one or more target antigens. In some embodiments, the antigen-binding moiety includes one or more antigen-binding determinants of an antibody or a functional antigen-binding fragment thereof. One skilled in the art upon reading the present disclosure will readily understand that the term “functional fragment thereof” or “functional variant thereof” refers to a molecule having quantitative and/or qualitative biological activity in common with the wild-type molecule from which the fragment or variant was derived. For example, a functional fragment or a functional variant of an antibody is one which retains essentially the same ability to bind to the same epitope as the antibody from which the functional fragment or functional variant was derived. For instance, an antibody capable of binding to an epitope of a cell surface receptor may be truncated at the N-terminus and/or C-terminus, and the retention of its epitope binding activity assessed using assays known to those of skill in the art. In some embodiments, the antigen-binding moiety is selected from the group consisting of an antibody, a nanobody, a diabody, a triabody, or a minibody, an F(ab′)2 fragment, an F(ab) fragment, a single chain variable fragment (scFv), and a single domain antibody (sdAb), or a functional fragment thereof. In some embodiments, the antigen-binding moiety includes an scFv.

The antigen-binding moiety can include naturally-occurring amino acid sequences or can be engineered, designed, or modified to provide desired and/or improved properties such as, e.g., binding affinity. Generally, the binding affinity of an antigen-binding moiety, e.g., an antibody, for a target antigen (e.g., CD19 antigen) can be calculated by the Scatchard method described by Frankel et al., Mol. Immunol, 16:101-06, 1979. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. An antibody that “selectively binds” an antigen (such as CD19) is an antigen-binding moiety that does not significantly bind other antigens but binds the antigen with high affinity, e.g., with an equilibrium constant (K_{D}) of 100 nM or less, such as 60 nM or less, for example, 30 nM or less, such as, 15 nM or less, or 10 nM or less, or 5 nM or less, or 1 nM or less, or 500 pM or less, or 400 pM or less, or 300 pM or less, or 200 pM or less, or 100 pM or less.

A skilled artisan can select an ECD based on the desired localization or function of a cell that is genetically modified to express a chimeric polypeptide or synZTE-containing Notch receptor of the present disclosure. For example, a chimeric polypeptide or synZTE-containing Notch receptor with an ECD including an antibody specific for a HER2 antigen can target cells to HER2-expressing breast cancer cells. In some embodiments, the ECD of the synZTE-containing Notch receptors disclosed herein is capable of binding a tumor-associated antigen (TAA) or a tumor-specific antigen (TSA). A skilled artisan will understand that TAAs include a molecule, such as e.g., protein, present on tumor cells and on normal cells, or on many normal cells, but at much lower concentration than on tumor cells. In contrast, TSAs generally include a molecule, such as e.g., protein which is present on tumor cells but absent from normal cells.

In some embodiments of the disclosure, the antigen-binding moiety of the ECD is specific for an epitope present in an antigen that is expressed by a tumor cell, i.e., a tumor-associated antigen. The tumor-associated antigen can be an antigen associated with, e.g., a breast cancer cell, a B cell lymphoma, a pancreatic cancer, a Hodgkin lymphoma cell, an ovarian cancer cell, a prostate cancer cell, a mesothelioma, a lung cancer cell, a non-Hodgkin B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a neuroblastoma cell, a glioma, a glioblastoma, a colorectal cancer cell, etc. It will also be understood that a tumor-associated antigen may also be expressed by a non-cancerous cell. In some embodiments, the antigen-binding domain is specific for an epitope present in a tissue-specific antigen. In some embodiments, the antigen-binding domain is specific for an epitope present in a disease-associated antigen.

Non-limiting examples of suitable target antigens include CD19, B7H3 (CD276), BCMA(CD269), alkaline phosphatase placental-like 2 (ALPPL2), green fluorescent protein (GFP), enhanced green fluorescent protein (EGFP), and signal regulatory protein α (SIRPα), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD 117), MUC1, NCAM, PAP, PDGFR-β, PRSS21, PSCA, PSMA, ROR1, SSEA-4, TAG72, TEM1/CD248, TEM7R, TSHR, VEGFR2, ALPI, citrullinated vimentin, cMet, and Axl.

In some embodiments, the target antigen is selected from CD19, B7H3 (CD276), BCMA (CD269), CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Ra, KIT (CD117), MUC1, NCAM, PAP, PDGFR-β, PRSS21, PSCA, PSMA, ROR1, SSEA-4, TAG72, TEM1/CD248, TEM7R, TSHR, VEGFR2, ALPI, citrullinated vimentin, cMet, Axl, GPC2, human epidermal growth factor receptor 2 (Her2/neu), CD276 (B7H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1, AOC3 (VAP-1), CAM-3001, CCL11 (eotaxin-1), CD125, CD147 (basigin), CD154 (CD40L), CD2, CD20, CD23 (IgE receptor), CD25 (a subunit of the heterodimeric IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4p7, LFA-1 (CD11α), myostatin, OX-40, scleroscin, SOST, TGFβ1, TNF-α, VEGF-A, pyruvate kinase isoenzyme type M2 (tumor M2-PK), CD20, CD5, CD7, CD3, TRBC1, TRBC2, BCMA, CD38, CD123, CD93, CD34, CD1α, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin.

Further non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β (CD61), galactin, K-Ras (V-Ki-ras2 Kirsten rat sarcoma viral oncogene), Ral-B, GPC2, CD276 (B7H3), or IL-13Rα. In some embodiments, the antigen includes ALPPL2. In some embodiments, the antigen includes BCMA. In some embodiments, the antigen-binding moiety of the ECD is specific for a reporter protein, such as GFP and eGFP. Non-limiting examples of such antigen binding moiety include a LaG17 anti-GFP nanobody. In some embodiments, the antigen-binding moiety of the ECD includes an anti-BCMA fully-humanized VH domain (FHVH). In some embodiments, the antigen includes signal regulatory protein α (SIRPα).

Additional antigens suitable for targeting by the chimeric receptors disclosed herein include, but are not limited to GPC2, human epidermal growth factor receptor 2 (Her2/neu), CD276 (B7H3), IL-13Rα1, IL-13Rα2, α-fetoprotein (AFP), carcinoembryonic antigen (CEA), cancer antigen-125 (CA-125), CA19-9, calretinin, MUC-1, epithelial membrane protein (EMA), epithelial tumor antigen (ETA). Other suitable target antigens include, but are not limited to, tyrosinase, melanoma-associated antigen (MAGE), CD34, CD45, CD123, CD93, CD99, CD117, chromogranin, cytokeratin, desmin, glial fibrillary acidic protein (GFAP), gross cystic disease fluid protein (GCDFP-15), ALK, DLK1, FAP, NY-ESO, WT1, HMB-45 antigen, protein melan-A (melanoma antigen recognized by T lymphocytes; MART-1), myo-D1, muscle-specific actin (MSA), neurofilament, neuron-specific enolase (NSE), placental alkaline phosphatase, synaptophysin, thyroglobulin, thyroid transcription factor-1.

Additional antigens suitable for targeting by the chimeric receptors disclosed herein include, but are not limited to, those associated with an inflammatory disease such as, AOC3 (VAP-1), CAM-3001, CCL11 (eotaxin-1), CD125, CD147 (basigin), CD154 (CD40L), CD2, CD20, CD23 (IgE receptor), CD25 (a subunit of the heteromeric IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4p7, LFA-1 (CD11α), myostatin, OX-40, scleroscin, SOST, TGFβ1, TNF-α, and VEGF-A.

Further antigens suitable for targeting by the chimeric polypeptides and synZTE-containing Notch receptors disclosed herein include, but are not limited to the pyruvate kinase isoenzyme type M2 (tumor M2-PK), CD20, CD5, CD7, CD3, TRBC1, TRBC2, BCMA, CD38, CD123, CD93, CD34, CD1α, SLAMF7/CS1, FLT3, CD33, CD123, TALLA-1, CSPG4, DLL3, Kappa light chain, Lamba light chain, CD16/FcγRIII, CD64, FITC, CD22, CD27, CD30, CD70, GD2 (ganglioside G2), GD3, EGFRvIII (epidermal growth factor variant III), EGFR and isovariants thereof, TEM-8, sperm protein 17 (Sp17), mesothelin. Additional non-limiting examples of suitable antigens include PAP (prostatic acid phosphatase), prostate stem cell antigen (PSCA), prostein, NKG2D, TARP (T cell receptor gamma alternate reading frame protein), Trp-p8, STEAP1 (six-transmembrane epithelial antigen of the prostate 1), an abnormal ras protein, an abnormal p53 protein, integrin β3 (CD61), galactin, K-Ras (V-Ki-ras2 Kirsten rat sarcoma viral oncogene), and Ral-B. In some embodiments, the antigen is GPC2, CD19, Her2/neu, CD276 (B7H3), IL-13Rα1, or IL-13Rα2.

In some embodiments, antigens suitable for targeting by the chimeric polypeptides and synZTE-containing Notch receptors disclosed herein include ligands derived from a pathogen. For example, the antigen can be HER2 produced by HER2-positive breast cancer cells. In some embodiments, the antigen can be CD19 that is expressed on B-cell leukemia. In some embodiments, the antigen can be EGFR that is expressed on glioblastoma multiform (GBM) but much less expressed so on healthy CNS tissue. In some embodiments, the antigen can be CEA that is associated with cancer in adults, for example colon cancer.

In some embodiments, the ligand is selected from the group consisting of CD1, CD1α, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), enhanced green fluorescent protein (EGFP), and signal regulatory protein α (SIRPα). In some embodiments, the antigen-binding moiety of the ECD is specific for a cell surface target, where non-limiting examples of cell surface targets include CD19, CD30, Her2, CD22, ENPP3, EGFR, CD20, CD52, CD11α, and α-integrin. In some embodiments, the chimeric polypeptides and synZTE-containing Notch receptors disclosed herein include an ECD having an antigen-binding moiety that binds CD19, CEA, HER2, MUC1, CD20, or EGFR.

In some embodiments, the chimeric polypeptides and synZTE-containing Notch receptors disclosed herein include an ECD containing an antigen-binding moiety that binds CD19. In some embodiments, the ECD includes an amino acid sequence having at least 70%, at least 75%, at least 80%, at least 85%, at least 90%, at least 95%, at least 96%, at least 97%, at least 98%, at least 99% sequence identity to SEQ ID NO: 10 in the Sequence Listing. In some embodiments, the ECD includes an amino acid sequence having at least 90% sequence identity to SEQ ID NO: 10. In some embodiments, the ECD includes an amino acid sequence having at least 95% sequence identity to SEQ ID NO: 10. In some embodiments, the ECD includes an amino acid sequence having 100% sequence identity to SEQ ID NO: 10. In some embodiments, the ECD includes an amino acid sequence having a sequence selected from the group consisting of SEQ ID NO: 10, wherein one, two, three, four, or five of the amino acid residues in SEQ ID NO: 10 is/are substituted by a different amino acid residue.

Linking Polypeptide/JMD

As described in greater detail below, the Notch extracellular domains located N-terminally to the TMD of the chimeric polypeptide of the disclosure include a linking polypeptide sequence disposed between the extracellular binding domain (ECD) and the transmembrane domain (TMD). In some embodiments, the length and amino acid composition of the linking polypeptide sequence can be optimized to vary the orientation and/or proximity of ECD and TMD relative to one another to achieve a desired activity of the chimeric polypeptides and receptors as disclosed herein. In some embodiments, the length and amino acid composition of the linking polypeptide sequence can be varied as a “tuning” tool to achieve a tuning effect that would enhance or reduce the biological activity of the disclosed chimeric polypeptides and receptors. Additional information regarding the relative glycine content, length, amino acid composition, and the flexibility/stiffness of glycerin-serine linking polypeptide can be determined by any methodologies known in the art as suitable for such purposes, for example as determined by Förster resonance energy transfer (FRET) efficiencies as described in Rosmalen M. et al., Biochemistry (2017), 56:6565-74.

In some embodiments, an single-chain peptide including about two to 100 amino acid residues (aa) e.g., 4, 5, 6, 7, 8, 9, 10, 11, 12, 13, 14, 15, 16, 17, 18, 19, 20, etc. amino acid residues) is used as a linking polypeptide in the disclosed chimeric receptors. In some embodiments, the linking polypeptide sequence has a length ranging from about 5 to about 50, about 10 to about 60, about 20 to about 70, about 30 to about 80, about 40 to about 90, about 50 to about 100, about 60 to about 80, about 70 to about 100, about 30 to about 60, about 20 to about 80, about 30 to about 90 amino acid residues. In some embodiments, the linking polypeptide sequence has a length ranging from about 1 to about 10, about 5 to about 15, about 10 to about 20, about 15 to about 25, about 20 to about 40, about 30 to about 50, about 40 to about 60, about 50 to about 70 amino acid residues. In some embodiments, the linking polypeptide sequence has a length ranging from about 40 to about 70, about 50 to about 80, about 60 to 8 about 0, about 70 to about 90, or about 80 to about 100 amino acid residues. In some embodiments, the linking polypeptide sequence has a length ranging from about 1 to about 10, about 5 to about 15, about 10 to about 20, about 15 to about 25 amino acid residues.

In some embodiments, the linking polypeptide sequence has a length ranging from about 1 to about 40 amino acid residues. In some embodiments, the linking polypeptide sequence has a length ranging from 1 to about 10, about 5 to about 20, about 10 to about 30, about 15 to about 40, about 10 to about 40, about 15 to about 40, about 20 to about 40, about 25 to about 40, about 30 to about 40, about 5 to about 0, about 15 to about 30 amino acid residues. In some embodiments, the linking polypeptide sequence has a length ranging from about 5 to about 40, about 10 to about 35, about 15 to about 35, about 20 to about 35, about 5 to about 20, about 5 to about 25, about 5 to about 30, about 5 to about 35 amino acid residues.

In certain embodiments, the linking polypeptide contains only glycine and/or serine residues (e.g., glycine-serine linking polypeptide). Examples of such linking polypeptides include: Gly, Ser; Gly Gly Ser; Ser Gly Gly; Gly Ser Gly; Gly Gly Gly Ser (SEQ ID NO: 80); Ser Gly Gly Gly (SEQ ID NO: 81); Ser Gly Ser Gly (SEQ ID NO: 82); Gly Gly Gly Gly Ser (SEQ ID NO: 83); Ser Gly Gly Gly Gly (SEQ ID NO: 84); Gly Gly Gly Gly Gly Ser (SEQ ID NO: 85); Ser Gly Gly Gly Gly Gly (SEQ ID NO: 86); Gly Gly Gly Gly Gly Gly Ser (SEQ ID NO: 87); Ser Gly Gly Gly Gly Gly Gly (SEQ ID NO: 88); (Gly Gly Gly Gly Ser)n (SEQ ID NO: 89), wherein n is an integer of one or more; and (Ser Gly Gly Gly Gly)n (SEQ ID NO: 90), wherein n is an integer of one or more. In some embodiments, the linking polypeptide sequence includes at least one glycine residue. In some embodiments, the linking polypeptide sequence includes at least one serine residue. In some embodiments, the linking polypeptide sequences are modified such that the amino acid sequence Gly Ser Gly (GSG) (that occurs at the junction of traditional Gly/Ser linker polypeptide repeats) is not present. For example, in some embodiments, the linking polypeptide includes an amino acid sequence selected from the group consisting of: (GGGXX)nGGGGS (SEQ ID NO: 91) and GGGGS(XGGGS)n (SEQ ID NO: 92), where X is any amino acid that can be inserted into the sequence and not result in a polypeptide comprising the sequence GSG, and n is 0 to 4. In some embodiments, the sequence of a linking polypeptide is (GGGX1X2)nGGGGS (SEQ ID NO: 93) and X1 is P and X2 is S and n is 0 to 4. In some embodiments, the sequence of a linking polypeptide is (GGGX1X2)nGGGGS (SEQ ID NO: 94) and X1 is G and X2 is Q and n is 0 to 4. In some other embodiments, the sequence of a linking polypeptide is (GGGX1X2)nGGGGS (SEQ ID NO: 95) and X1 is G and X2 is A and n is 0 to 4. In some embodiments, the sequence of a linking polypeptide is GGGGS(XGGGS)n (SEQ ID NO: 96), and X is P and n is 0 to 4. In some embodiments, a linking polypeptide of the disclosure comprises or consists of the amino acid sequence (GGGGA)₂GGGGS (SEQ ID NO: 97). In some embodiments, a linking polypeptide comprises or consists of the amino acid sequence (GGGGQ)₂GGGGS (SEQ ID NO: 98). In some embodiments, a linking polypeptide comprises or consists of the amino acid sequence (GGGPS)₂GGGGS (SEQ ID NO: 99). In some embodiments, a linking polypeptide comprises or consists of the amino acid sequence GGGGS(PGGGS)₂ (SEQ ID NO: 100).

In some embodiments, a linking polypeptide the amino acid sequence (GGS)n wherein n is an integer from 1 to 50, for example, from 1 to 10, from 5 to 15, from 10 to 20, from 15 to 25, from 20 to 30, from 25 to 35, from 30 to 40, from 35 to 45, or from 40 to 50. In some embodiments, a linking polypeptide the amino acid sequence (GGS)n wherein n is an integer from 1 to 10. In some embodiments, a linking polypeptide the amino acid sequence (GGS)n wherein n is an integer from 10 to 20. In some embodiments, a linking polypeptide the amino acid sequence (GGS)n wherein n is an integer from 20 to 30. In some embodiments, a linking polypeptide the amino acid sequence (GGS)n wherein n is an integer from 30 to 40. In some embodiments, a linking polypeptide the amino acid sequence (GGS)n wherein n is an integer from 40 to 50. In some embodiments, n is 18, i.e., (GGS)₁₈. In some embodiments, n is 15, i.e., (GGS)₁₅. In some embodiments, n is 12, i.e., (GGS)₁₂. In some embodiments, n is 9, i.e., (GGS)₉. In some embodiments, n is 6, i.e., (GGS)₆. In some embodiments, n is 3, i.e., (GGS)₃. In some embodiments, the sequence is selected so that it does not include a common protease cleavage site. In some embodiments, the sequence is selected so that it does not include a glycosylation site.

In some embodiments, a linking polypeptide comprises or consists of an amino acid sequence having at least 80% sequence identity, such as, at least 80%, at least 85%, at least 90%, at least 95%, at least 96%, at least 97%, at least 98%, or 99% sequence identity to a sequence set forth in SEQ ID NOS: 25-28 in the Sequence Listing. In some embodiments, a linking polypeptide comprises or consists of an amino acid sequence having at least 80% sequence identity to a sequence set forth in SEQ ID NOS: 25-28. In some embodiments, a linking polypeptide comprises or consists of an amino acid sequence having at least 90% sequence identity to a sequence set forth in SEQ ID NOS: 25-28. In some embodiments, a linking polypeptide comprises or consists of an amino acid sequence having at least 95% sequence identity to a sequence set forth in SEQ ID NOS: 25-28. In some embodiments, a linking polypeptide comprises or consists of an amino acid sequence having about 100% sequence identity to a sequence set forth in SEQ ID NOS: 25-28. In some embodiments, a linking polypeptide comprises or consists of an amino acid sequence selected from the group consisting of SEQ ID NOS: 25-28, wherein one, two, three, four, or five of the amino acid residues in any one of SEQ ID NOS: 25-28 is/are substituted by a different amino acid residues.

In some embodiments, the linking polypeptide has substantial sequence identity with a Notch receptor JMD which is partly or completely devoid of the NRR and/or the HD. In some embodiments, the linking polypeptide has at least about 80%, 85%, 90%, 95%, 96%, 97%, 98%, 99%, or 100% sequence identity to a Notch JMD wherein a LNR and/or a HD of a Notch receptor has been deleted. In some embodiments, the linking polypeptide has at least about 80%, 85%, 90%, 95%, 96%, 97%, 98%, 99%, or 100% sequence identity to a Notch JMD wherein at least one LNR of a Notch receptor has been deleted. In some embodiments, the linking polypeptide has at least about 80%, 85%, 90%, 95%, 96%, 97%, 98%, 99%, or 100% sequence identity to a Notch JMD wherein at least two LNRs of a Notch receptor has been deleted. In some embodiments, the linking polypeptide has at least about 80%, 85%, 90%, 95%, 96%, 97%, 98%, 99%, or 100% sequence identity to a Notch JMD wherein one, or two, or all three LNRs of a Notch receptor has been deleted. In some embodiments, the linking polypeptide has at least about 80%, 85%, 90%, 95%, 96%, 97%, 98%, 99%, or 100% sequence identity to a Notch JMD which does not include a Notch NRR or HD of a Notch receptor, e.g., complete absence of the Notch extracellular subunit (NEC).

In some embodiments, the linker polypeptide sequence includes a sequence having at least 80% sequence identity, such as, at least 80%, at least 85%, at least 90%, at least 95%, at least 96%, at least 97%, at least 98%, or 99% sequence identity to a N-JMD sequence selected from the group consisting of SEQ ID NOS: 11-18 in the Sequence Listing. In some embodiments, the linker polypeptide sequence includes an amino acid sequence having at least 90% sequence identity to a N-JMD domain selected from the group consisting of SEQ ID NOS: 11-18. In some embodiments, the linker polypeptide sequence includes an amino acid sequence having at least 95% sequence identity to a N-JMD domain selected from the group consisting of SEQ ID NOS: 19-27. In some embodiments, the linker polypeptide sequence includes an amino acid sequence having about 100% sequence identity to a N-JMD domain selected from the group consisting of SEQ ID NOS: 19-27. In some embodiments, the linker polypeptide sequence includes a N-JMD domain having a sequence selected from the group consisting of SEQ ID NOS: 11-18, wherein one, two, three, four, or five of the amino acid residues in any one of the SEQ ID NOS: 11-18 is/are substituted by a different amino acid residue.

Hinge Domain

As described in greater detail herein, the linking polypeptide of the chimeric Notch receptors in accordance with some embodiments of the disclosure incorporate an extracellular oligomerization domain to promote formation of oligomeric forms, e.g., dimeric or trimeric form of the chimeric receptors. It is believed, without being bound by any theory, that this design allows oligomerization/clustering of extracellular domains (ECD) and subsequently brings together intracellular domains (ICD) to activate cell signaling, e.g. T-cell signaling. In these instances, the Notch ECDs located N-terminally to the TMD include an oligomerization domain (e.g., a polypeptide hinge domain) containing one or more polypeptide motifs that promote oligomer formation of the chimeric polypeptides via intermolecular disulfide bonding. In these instances, the hinge domain generally includes a flexible oligo- or polypeptide connector region disposed between the ECD and the TMD. Thus, the polypeptide hinge domain provides flexibility between the ECD and TMD and also provides sites for intermolecular disulfide bonding between two or more chimeric polypeptide monomers to form an oligomeric complex. In some embodiments, the hinge domain includes motifs that promote dimer formation of the chimeric polypeptides disclosed herein. In some embodiments, the hinge domain includes motifs that promote trimer formation of the chimeric polypeptides disclosed herein (e.g., a hinge domain derived from OX40).

Hinge polypeptide sequences suitable for the compositions and methods of the disclosure can be naturally-occurring hinge polypeptide sequences (e.g., those from naturally-occurring immunoglobulins). Alternatively, a hinge polypeptide sequence can be a synthetic sequence that corresponds to a naturally-occurring hinge polypeptide sequence, or can be an entirely synthetic hinge sequence, or can be engineered, designed, or modified to provide desired and/or improved properties, e.g., modulating transcription. Suitable hinge polypeptide sequences include, but are not limited to, those derived from IgA, IgD, and IgG subclasses, such as IgG1 hinge domain, IgG2 hinge domain, IgG3 hinge domain, and IgG4 hinge domain, or a functional variant thereof. In some embodiments, the hinge polypeptide sequence contains one or more CXXC motifs. In some embodiments, the hinge polypeptide sequence contains one or more CPPC motifs. Additional information in this regard can be found in, for example, a recent review by Vidarsson G. et al., Frontiers Immunol. (Oct. 20, 2014) 5:520, which is hereby incorporated by reference in its entirety.

Accordingly, in some embodiments, the hinge domain of the chimeric Notch receptors disclosed herein includes a hinge polypeptide sequence derived from an IgG1 hinge domain or a functional variant thereof. In some embodiments, the hinge domain includes a hinge polypeptide sequence derived from an IgG2 hinge domain or a functional variant thereof. In some embodiments, the hinge domain includes a hinge polypeptide sequence derived from an IgG3 hinge domain or a functional variant thereof. In some embodiments, the hinge domain includes a hinge polypeptide sequence derived from an IgG4 hinge domain or a functional variant thereof. In some embodiments, the hinge domain includes a hinge polypeptide sequence derived from an IgA hinge domain or a functional variant thereof. In some embodiments, the hinge domain includes a hinge polypeptide sequence derived from an IgD hinge domain or a functional variant thereof.

Additional hinge polypeptide sequences suitable for the compositions and methods disclosed herein include, but are not limited to, hinge polypeptide sequences derived from a CD8α hinge domain, a CD28 hinge domain, a CD152 hinge domain, a PD-1 hinge domain, a CTLA4 hinge domain, an OX40 hinge domain, an FcγRIIIα hinge domain, and functional variants thereof. In some embodiments, the hinge domain includes a hinge polypeptide sequence derived from a CD8α hinge domain or a functional variant thereof. In some embodiments, the hinge domain includes a hinge polypeptide sequence derived from a CD28 hinge domain or a functional variant thereof. In some embodiments, the hinge domain includes a hinge polypeptide sequence derived from an OX40 hinge domain or a functional variant thereof. In some embodiments, the hinge domain includes a hinge polypeptide sequence derived from an IgG4 hinge domain or a functional variant thereof.

The length and/or amino acid composition of the hinge domain are selected to confer flexibility and the capacity for oligomerization. One skilled in the art will readily appreciate that the length and amino acid composition of the hinge polypeptide sequence can be optimized to vary the orientation and/or proximity of the ECD and the TMD relative to one another, as well as of the chimeric polypeptide monomers to one another, to achieve a desired activity of the chimeric polypeptide of the disclosure. In some embodiments, a single-chain peptide including about one to 100 amino acid residues (e.g., 2, 3, 4, 5, 6, 7, 8, 9, 10, 11, 12, 13, 14, 15, 16, 17, 18, 19, 20, etc. amino acid residues) can be used as a hinge domain. In some embodiments, the hinge domain includes about 5 to 50, about 10 to 60, about 20 to 70, about 30 to 80, about 40 to 90, about 50 to 100, about 60 to 80, about 70 to 100, about 30 to 60, about 20 to 80, about 30 to 90 amino acid residues. In some embodiments, the hinge domain includes about 1 to 10, about 5 to 15, about 10 to 20, about 15 to 25, about 20 to 40, about 30 to 50, about 40 to 60, about 50 to 70 amino acid residues. In some embodiments, the hinge domain includes about 40 to 70, about 50 to 80, about 60 to 80, about 70 to 90, or about 80 to 100 amino acid residues. In some embodiments, the hinge domain includes about 1 to 10, about 5 to 15, about 10 to 20, about 15 to 25 amino acid residues. In some embodiments, the hinge domain includes a sequence having at least 80% sequence identity, such as, at least 80%, at least 85%, at least 90%, at least 95%, at least 96%, at least 97%, at least 98%, or 99% sequence identity to a sequence selected from the group consisting of SEQ ID NOS: 20-24 in the Sequence Listing. In some embodiments, the hinge domain includes an amino acid sequence having at least 90% sequence identity to a sequence selected from the group consisting of SEQ ID NOS: 20-24. In some embodiments, the hinge domain includes an amino acid sequence having at least 95% sequence identity to a sequence selected from the group consisting of SEQ ID NOS: 20-24. In some embodiments, the hinge domain includes an amino acid sequence having about 100% sequence identity to a sequence selected from the group consisting of SEQ ID NOS: 20-24. In some embodiments, the hinge domain includes an amino acid sequence having a sequence selected from the group consisting of SEQ ID NOS: 20-24, wherein one, two, three, four, or five of the amino acid residues in any one of the SEQ ID NOS: 20-24 is/are substituted by a different amino acid residue.

Transmembrane Domain (TMD)

As described in greater detail herein, the chimeric polypeptides and the engineered Notch receptors of the disclosure include a transmembrane domain having at least about 80% sequence identity to the TMD of a Type 1 transmembrane receptor and including one or more ligand-inducible proteolytic cleavage sites.

Examples of ligand-inducible proteolytic cleavage sites in a Notch receptor (e.g., S2 or S3) are as described above. Additional proteolytic cleavage sites suitable for the compositions and methods disclosed herein include, but are not limited to, a metalloproteinase cleavage site for a matrix metalloproteinase (MMP) selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (SEQ ID NO: 101) (wherein, X represents any residue; Hy, a hydrophobic residue), e.g., Pro-X-X-Hy-(Ser/Thr) (SEQ ID NO: 102), e.g., Pro-Leu/Gln-Gly-Met-Thr-Ser (SEQ ID NO: 103) or Pro-Leu/Gln-Gly-Met-Thr (SEQ ID NO: 104). Another example of a suitable protease cleavage site is a plasminogen activator cleavage site, e.g., a urokinase plasminogen activator (uPA) or a tissue plasminogen activator (tPA) cleavage site. Another example of a suitable protease cleavage site is a prolactin cleavage site. Specific examples of cleavage sequences of uPA and tPA include sequences including Val-Gly-Arg (SEQ ID NO: 105). Another example of a protease cleavage site that can be included in a proteolytically cleavable linker is a tobacco etch virus (TEV) protease cleavage site, e.g., Glu-Asn-Leu-Tyr-Thr-Gln-Ser (SEQ ID NO: 106), where the protease cleaves between the glutamine and the serine. Another example of a protease cleavage site that can be included in a proteolytically cleavable linker is an enterokinase cleavage site, e.g., Asp-Asp-Asp-Asp-Lys (SEQ ID NO: 107), where cleavage occurs after the lysine residue. Another example of a protease cleavage site that can be included in a proteolytically cleavable linker is a thrombin cleavage site, e.g., Leu-Val-Pro-Arg (SEQ ID NO: 108). Additional suitable linkers including protease cleavage sites include sequences cleavable by the following proteases: a PreScission™ protease (a fusion protein including human rhinovirus 3C protease and glutathione-S-transferase), a thrombin, cathepsin B, Epstein-Barr virus protease, MMP-3 (stromelysin), MMP-7 (matrilysin), MMP-9; thermolysin-like MMP, matrix metalloproteinase 2 (MMP-2), cathepsin L; cathepsin D, matrix metalloproteinase 1 (MMP-1), urokinase-type plasminogen activator, membrane type 1 matrix metalloproteinase (MT-MMP), stromelysin 3 (or MMP-11), thermolysin, fibroblast collagenase and stromelysin-1, matrix metalloproteinase 13 (collagenase-3), tissue-type plasminogen activator (tPA), human prostate-specific antigen, kallikrein (hK3), neutrophil elastase, and calpain (calcium activated neutral protease). Proteases that are not native to the cell in which the receptor is expressed (for example, TEV) can be used as a further regulatory mechanism, in which activation of the synthetic Notch receptor of the disclosure is reduced until the protease is expressed or otherwise provided. Additionally, a protease may be tumor-associated or disease-associated (expressed to a significantly higher degree than in normal tissue), and serve as an independent regulatory mechanism. For example, some matrix metalloproteases are highly expressed in certain cancer types.

Generally, the TMD suitable for the chimeric receptors disclosed herein can be any transmembrane domain of a Type 1 transmembrane receptor including at least one γ-secretase cleavage site. Detailed description of the structure and function of the γ-secretase complex as well as its substrate proteins, including amyloid precursor protein (APP) and Notch, can, for example, be found in a recent review by Zhang et al., Frontiers Cell Neurosci (2014). Non-limiting suitable TMDs from Type 1 transmembrane receptors include those from CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, and IFNAR2, wherein the TMD includes at least one γ-secretase cleavage site. Additional TMDs suitable for the compositions and methods described herein include, but are not limited to, transmembrane domains from Type 1 transmembrane receptors IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM. In some embodiments, the TMD of the chimeric polypeptides or Notch receptors of the disclosure is a TMD derived from the TMD of a member of the calsyntenin family, such as, alcadein alpha and alcadein gamma. In some embodiments, the TMD of the chimeric polypeptides or Notch receptors of the disclosure is a TMD derived from a different Notch receptor. For example, in a synthetic Notch receptor based on human Notch1, the Notch1 TMD can be substituted with a human Notch2 TMD, human Notch3 TMD, human Notch4 TMD, or a Notch TMD from a non-human animal such as Danio rerio, Drosophila melanogaster, Xenopus laebis, or Gallus.

Accordingly, in some embodiments, the transmembrane domain includes an amino acid sequence exhibiting at least 70%, at least 75%, at least 80%, at least 85%, at least 90%, at least 95%, at least 96%, at least 97%, at least 98%, at least 99% sequence identity to a polypeptide sequence having at least about 70% sequence identity to a transmembrane domain from a Type 1 transmembrane receptor that includes a γ-secretase cleavage site. In some embodiments, the transmembrane domain includes an amino acid sequence exhibiting at least 70% sequence identity to a transmembrane domain from a Type 1 transmembrane receptor that includes a γ-secretase cleavage site. In some embodiments, the transmembrane domain includes an amino acid sequence exhibiting at least about 80% sequence identity to a transmembrane domain from a Type 1 transmembrane receptor that includes a γ-secretase cleavage site. In some embodiments, the transmembrane domain includes an amino acid sequence exhibiting at least about 90% sequence identity to a transmembrane domain from a Type 1 transmembrane receptor that includes a γ-secretase cleavage site. In some embodiments, the transmembrane domain includes an amino acid sequence exhibiting at least about 95% sequence identity to a transmembrane domain from a Type 1 transmembrane receptor that includes a γ-secretase cleavage site. In some embodiments, the Type 1 transmembrane receptor is selected from the group consisting of CLSTN1, CLSTN2, APLP1, APLP2, LRP8, APP, BTC, TGBR3, SPN, CD44, CSF1R, CXCL16, CX3CL1, DCC, DLL1, DSG2, DAG1, CDH1, EPCAM, EPHA4, EPHB2, EFNB1, EFNB2, ErbB4, GHR, HLA-A, IFNAR2, IL1R1, IL1R2, IL6R, INSR, ERN1, ERN2, JAG2, KCNE1, KCNE2, KCNE3, KCNE4, KL, CHL1, PTPRF, SCN1B, SCN3B, NPR3, NGFR, PLXDC2, PAM, AGER, ROBO1, SORCS3, SORCS1, SORL1, SDC1, SDC2, SPN, TYR, TYRP1, DCT, VASN, FLT1, CDH5, PKHD1, NECTIN1, PCDHGC3, NRG1, LRP1B, CDH2, NRG2, PTPRK, SCN2B, Nradd, and PTPRM.

In some embodiments, the TMD includes an amino acid sequence exhibiting at least 70%, at least 75%, at least 80%, at least 85%, at least 90%, at least 95%, at least 96%, at least 97%, at least 98%, at least 99% sequence identity to one or more of SEQ ID NOS: 29-38 in the Sequence Listing. In some embodiments, the transmembrane domain includes an amino acid sequence having at least 90% sequence identity to a sequence selected from the group consisting of SEQ ID NOS: 29-38. In some embodiments, the transmembrane domain includes an amino acid sequence having at least 95% sequence identity to a sequence selected from the group consisting of SEQ ID NOS: 29-38. In some embodiments, the transmembrane domain includes an amino acid sequence having about 100% sequence identity to one or more of SEQ ID NOS: 29-38. In some embodiments, the transmembrane domain includes an amino acid sequence having a sequence selected from the group consisting of SEQ ID NOS: 29-38, wherein one, two, three, four, or five of the amino acid residues in any one of the SEQ ID NOS: 29-38 is/are substituted by a different amino acid residue.

In some embodiments, the amino acid substitution(s) within the TMD includes one or more substitutions within a “GV” motif of the TMD. In some embodiments, at least one of such substitution(s) is a substitution to alanine.

Stop-Transfer-Sequence (STS)

In some embodiments, the chimeric polypeptides and synZTE-containing Notch receptors of the disclosure include a stop-transfer-sequence (STS) which constitutes a highly-charged domain located C-terminally to the TMD. Without being bound to any particular theory, such a highly-charged domain disposed between the TMD and the ICD prevents the ICD from entering the membrane. The STS is linked to the TMD and the ICD in the following order, from N-terminus to C-terminus, TMD-STS-ICD. The length and/or amino acid composition of the STS can be selected to achieve the desired receptor sensitivity. In some embodiments, a single-chain peptide including about 4 to about 40 amino acid residues (e.g., 4, 5, 6, 7, 8, 9, 10, 11, 12, 13, 14, 15, 16, 17, 18, 19, or 20, etc. amino acid residues) can be used as a STS. In some embodiments, the STS includes about 4 to 15, about 6 to 20, about 8 to 25, about 10 to 30, about 12 to 35, about 14 to 40, about 5 to 40, about 10 to 35, about 15 to 30, about 20 to 25, about 20 to 40, about 10 to 30, about 4 to 20, or about 5 to 25 amino acid residues. In some embodiments, the STS includes about 4 to 10, about 5 to 12, about 6 to 14, about 7 to 18, about 8 to 20, about 9 to 22, about 10 to 24, or about 11 to 26 amino acid residues. In some embodiments, the STS includes about 4 to 10 residues, such as, 4, 5, 6, 7, 8, 9, or 10 amino acid residues.

In some embodiments, the STS comprises a sequence having at least 70% sequence identity, such as, at least 70%, at least 75%, at least 80%, at least 85%, at least 90%, at least 95%, at least 96%, at least 97%, at least 98%, or 99% sequence identity to a STS sequence from Notch1, Notch2, Notch3, Notch4, CSF1R, CXCL16, DAG1, GHR, PTPRF, AGER, KL, NRG1, LRP1B, Jag2, EPCAM, KCNE3, CDH2, NRG2, PTPRK, BTC, EPHA3, IL1R2, or PTPRM. In some embodiments, the STS comprises a sequence comprising only Lys (K) or Arg (R) in the first 4 residues. In some embodiments, the STS comprises one, two, three, four, five, or more basic residues. In some embodiments, the STS comprises five, four, three, two, one, or zero aromatic residues or residues with hydrophobic and/or bulky side chains.

In some embodiments, the STS includes a sequence having at least 80% sequence identity, such as, at least 80%, at least 85%, at least 90%, at least 95%, at least 96%, at least 97%, at least 98%, or 99% sequence identity to a sequence selected from the group consisting of SEQ ID NOS: 39-54 in the Sequence Listing. In some embodiments, the STS includes an amino acid sequence having at least 90% sequence identity to a sequence selected from the group consisting of SEQ ID NOS: 39-54. In some embodiments, the STS includes an amino acid sequence having at least 95% sequence identity to a sequence selected from the group consisting of SEQ ID NOS: 39-54. In some embodiments, the STS includes an amino acid sequence having about 100% sequence identity to a sequence selected from the group consisting of SEQ ID NOS: 39-54. In some embodiments, the STS includes an amino acid sequence having a sequence selected from the group consisting of SEQ ID NOS: 39-54, wherein one, two, three, four, or five of the amino acid residues in any one of the SEQ ID NOS: 39-54 is/are substituted by a different amino acid residue.

Intracellular Domain

The chimeric polypeptides and engineered Notch receptors of the disclosure include a transcriptional effector. The transcriptional effector of the disclosure is a polypeptide element that acts to activate or inhibit the transcription of a promoter-driven DNA sequence. Transcriptional effectors suitable for the compositions and methods of the disclosure can be naturally-occurring transcriptional regulators or can be engineered, designed, or modified so as to provide desired and/or improved properties, e.g., modulating transcription. As discussed above, the engineered Notch receptors of the present disclosure are advantageous in that they can provide the ability to trigger a custom transcriptional program in engineered cells. In some embodiments, transcriptional effector of the disclosure is a custom transcriptional regulator that drives transcription off a specific sequence that only appears once in the engineered cell.

In some embodiments, the engineered Notch receptors of the disclosure include a zinc finger-containing transcriptional effector (ZTE) which includes one or more zinc finger motifs (ZF). A ZF is a finger-shaped fold in a protein that permits it to interact with nucleic acid sequences such as DNA and RNA. Such finger-shaped fold is well known in the art. The fold is generally created by the binding of specific amino acids in the protein to a zinc atom, and is stabilized by the co-ordination of a zinc ion between four largely invariant (depending on zinc finger framework type) Cys and/or His residues.

The term “motif” as used herein refers to a structural motif. A ZF motif is a relatively small polypeptide domain having a supersecondary structure, and includes approximately 30 amino acids and folds to form an α-helix adjacent an antiparallel β-sheet (known as a ββα-fold), and is stabilized by a zinc ion. A ZF domain recognizes and binds to a nucleic acid triplet, or an overlapping quadruplet (as explained below), in a double-stranded DNA target sequence. Naturally-occurring zinc finger domains (also known as ZF proteins) have been well studied and described in the literature. Natural ZF proteins can regulate the expression of genes as well as nucleic acid recognition, reverse transcription and virus assembly. Additional information in this regard can be found in, for example, U.S. Pat. No. 10,138,493, which is expressly incorporated herein by reference.

C₂H₂ zinc fingers (C₂H₂-ZFs) are among the most prevalent type of vertebrate DNA-binding domain, and generally appear in tandem arrays (ZFAs), with sequential C₂H₂-ZFs each contacting three (or more) sequential bases. C₂H₂-ZFs can be assembled in a modular fashion. Given a set of modules with defined three-base specificities, modular assembly also presents a way to construct artificial proteins with specific DNA-binding preferences.

ZF-containing proteins generally contain strings or chains of ZF motifs, forming an array of ZF (ZFA). Thus, a ZF protein may include two or more ZFs, e.g., a ZFA consisting of 2 or more ZF motifs, which may be directly adjacent one another (e.g., separated by a short linker sequence), or may be separated by longer, flexible or structured polypeptide sequences. For example, a ZFA can have six ZF motifs (a 6-finger ZFA), seven ZF motifs (a 7-finger ZFA), or eight ZF motifs (an 8-finger ZFA), arranged in tandem. Directly adjacent ZF domains are generally expected to bind to contiguous nucleic acid sequences, e.g., to adjacent trinucleotides/triplets. In some cases, cross-binding may also occur between adjacent ZF and their respective target triplets, which may help to strengthen or enhance the recognition of the target sequence, and leads to the binding of overlapping quadruplet sequences. By comparison, distant ZF domains within the same protein may recognize, and/or bind to, non-contiguous nucleic acid sequences or even to different molecules (e.g., protein rather than nucleic acid).

In some embodiments, the engineered Notch receptors of the disclosure include a zinc finger-containing transcriptional effector (ZTE) having a DNA binding zinc finger protein domain (ZF protein domain) and another domain through which the protein exerts its effect (effector domain). As described in further detail below, exemplary effector domains suitable for the engineered Notch receptors of the disclosure include, but are not limited to, transcriptional activating domains, transcriptional repressor domains, epigenetic effector domains, and DNA modifying enzymes.

In some embodiments, the engineered Notch receptors of the disclosure include a ZTE with two or more, e.g., 3 or more, for example, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more (e.g., up to approximately 30 or 32) ZF motifs arranged adjacent one another in tandem, forming arrays of ZF motifs or ZFA. In some embodiments, the ZTE includes at least 3 ZF motifs, at least 4 ZF motifs, at least 5 ZF motifs, or at least 6 ZF motifs, at least 7 ZF motifs, at least 8 ZF motifs, at least 9 ZF motifs, at least 10 ZF motifs, at least 11 or at least 12 ZF motifs; and in some cases at least 18 ZF motifs. In some embodiments, the ZTE of the engineered Notch receptors disclosed herein contains up to 6, 7, 8, 10, 11, 12, 16, 17, 18, 22, 23, 24, 28, 29, 30, 34, 35, 36, 40, 41, 42, 46, 47, 48, 54, 55, 56, 58, 59, or 60 ZF motifs. In some embodiments, the ZTE of the disclosure bind to orthogonal target nucleic acid binding sites. That is, the ZFs or ZFAs in ZF domain of the ZTE binds orthogonal target nucleic acid sequences. In some embodiments, the orthogonal target nucleic acid binding sites are contiguous. In some embodiments, the ZTE of the engineered Notch receptors disclosed herein binds target orthogonal specific DNA sequences and have, for example, reduced or minimal functional binding potential in a eukaryotic genome.

In some embodiments of the disclosure, the ZTE includes: (a) a first domain including a DNA-binding zinc finger protein domain (ZF protein domain), and (b) a second domain through which the ZTE exerts its effect (effector domain), wherein the ZTE has the following formula I:

[effector domain]ₐ-[ZF protein domain]-[effector domain]_{b} (Formula I)

wherein a and b are each independently an integer from 0 to 5, and at least one of a and b is not 0; wherein the ZF protein domain includes 1 to about 10 zinc finger arrays (ZFA); wherein the ZFA includes about 6 to about 8 zinc finger motifs according to formula II (from N-terminal to C-terminal):

X_{c}CX_{d}CXₑ-(helix)-HX_{f}H-L² (Formula II)

wherein L² is a linker peptide having about 4-6 amino acid residues, C is Cys, H is His, each X is independently any amino acid, c is an integer from 0 to 3, d is an integer from 1 to 5, e is an integer from 2 to 7, f is an integer from 3 to 6, and (helix) is a peptide domain of about 6 amino acids that forms an α-helix, wherein the ZFA is capable of binding a specific nucleic acid sequence.

In some embodiments, the ZF protein domain of the engineered Notch receptors disclosed herein includes 1 to about 10 ZFA, each of which independently includes a sequence having at least about 90% identity to a sequence selected from the group consisting of SEQ ID NOS: 55-60. In some embodiments, the ZFA includes a sequence having at least about 90%, at least about 95%, at least about 96%, at least about 97%, at least about 98%, or at least about 99% sequence identity to a sequence selected from the group consisting of SEQ ID NOS: 55-60. In some embodiments, the ZFA sequence has at least about 90%, at least about 95%, at least about 96%, at least about 97%, at least about 98%, or at least about 99% sequence identity to the sequence of SEQ ID NO: 55 (ZF2). In some embodiments, the ZFA sequence has at least about 90%, at least about 95%, at least about 96%, at least about 97%, at least about 98%, or at least about 99% sequence identity to the sequence of SEQ ID NO: 56 (ZF3). In some embodiments, the ZFA sequence has at least about 90%, at least about 95%, at least about 96%, at least about 97%, at least about 98%, or at least about 99% sequence identity to the sequence of SEQ ID NO: 57 (ZF4). In some embodiments, the ZFA sequence has at least about 90%, at least about 95%, at least about 96%, at least about 97%, at least about 98%, or at least about 99% sequence identity to the sequence of SEQ ID NO: 58 (ZF6). In some embodiments, the ZFA sequence has at least about 90%, at least about 95%, at least about 96%, at least about 97%, at least about 98%, or at least about 99% sequence identity to the sequence of SEQ ID NO: 59 (ZF10). In some embodiments, the ZFA sequence has at least about 90%, at least about 95%, at least about 96%, at least about 97%, at least about 98%, or at least about 99% sequence identity to the sequence of SEQ ID NO: 60 (ZF11).

In some embodiments, the ZF protein domain of the engineered Notch receptors disclosed herein includes 1 to about 10 ZFA, each of which independently includes a sequence having about 100% sequence identity to a sequence selected from the group consisting of SEQ ID NOS: 55-60. In some embodiments, the ZFA sequence has about 100% sequence identity to the sequence of SEQ ID NO: 55 (ZF2). In some embodiments, the ZFA sequence has about 100% sequence identity to the sequence of SEQ ID NO: 56 (ZF3). In some embodiments, the ZFA sequence has about 100% sequence identity to the sequence of SEQ ID NO: 57 (ZF4). In some embodiments, the ZFA sequence has about 100% sequence identity to the sequence of SEQ ID NO: 58 (ZF6). In some embodiments, the ZFA sequence has about 100% sequence identity to the sequence of SEQ ID NO: 59 (ZF10). In some embodiments, the ZFA sequence has about 100% sequence identity to the sequence of SEQ ID NO: 60 (ZF11).

In some embodiments, the ZF protein domain includes multiple ZFAs having the same amino acid sequences. In some embodiments, the ZF protein domain includes multiple ZFAs whose amino acid sequences are different from one another.

In some embodiments, the ZF protein domain of the engineered Notch receptors disclosed herein includes one or more ZFAs that are independently capable of specifically binding to a target nucleic acid sequence selected from the group consisting of SEQ ID NOS: 61-71. In some embodiments, at least one ZFA is capable of specifically binding to a target nucleic acid sequence having the sequence of SEQ ID NO: 61. In some embodiments, at least one ZFA is capable of specifically binding to a target nucleic acid sequence having the sequence of SEQ ID NO: 62. In some embodiments, at least one ZFA is capable of specifically binding to a target nucleic acid sequence having the sequence of SEQ ID NO: 63. In some embodiments, at least one ZFA is capable of specifically binding to a target nucleic acid sequence having the sequence of SEQ ID NO: 64. In some embodiments, at least one ZFA is capable of specifically binding to a target nucleic acid sequence having the sequence of SEQ ID NO: 65. In some embodiments, at least one ZFA is capable of specifically binding to a target nucleic acid sequence having the sequence of SEQ ID NO: 66. In some embodiments, at least one ZFA is capable of specifically binding to a target nucleic acid sequence having the sequence of SEQ ID NO: 67. In some embodiments, at least one ZFA is capable of specifically binding to a target nucleic acid sequence having the sequence of SEQ ID NO: 68. In some embodiments, at least one ZFA is capable of specifically binding to a target nucleic acid sequence having the sequence of SEQ ID NO: 69. In some embodiments, at least one ZFA is capable of specifically binding to a target nucleic acid sequence having the sequence of SEQ ID NO: 70. In some embodiments, at least one ZFA is capable of specifically binding to a target nucleic acid sequence having the sequence of SEQ ID NO: 71.

As described herein, the zinc finger-containing transcriptional effector (ZTE) of the engineered Notch receptors disclosed herein includes a second domain through which the ZTE exerts its effect (effector domain). Exemplary effector domains suitable for the engineered Notch receptors of the disclosure include, but are not limited to, transcriptional activating domains, transcriptional repressor domains, epigenetic effector domains, and DNA modifying enzymes.

Accordingly, in some embodiments, the effector domain of the ZTE includes a transcription-activating domain (TAD). Non-limiting examples of transcription-activating domains suitable for use in the compositions and methods disclosed herein include Herpes Simplex Virus Protein 16 (HSV VP16) activation domain; an activation domain consisting of four tandem copies of VP16 (VP64); a p65 activation domain of NFκB; an Epstein-Barr virus R transactivator activation domain (Rta); a tripartite activator consisting of VP64, and Rta activation domains (VPR); and a histone acetyltransferase core domain of the human E1A-associated protein p300 (p300 HAT core activation domain). In some embodiments, the effector domain of the ZTE includes a p65 activation domain of NFκB.

In some embodiments, the ZTE of the engineered Notch receptors disclosed herein is further operably linked to an additional heterologous sequence. The additional sequence can be fused to the transactivation domain of the ZTp65 TAD. In principle, the additional sequence can be derived from any suitable endogenous proteins. Non-limiting examples of suitable sequences include those derived from heat shock factor 1 (HSF1) or the glucocorticoid receptor (GR). In some aspects, the full protein glucocorticoid receptor contains multiple transactivation domains with their own subnames. As used herein in the chimeric polypeptides provided herein, any of the transactivation domain of GR can be used. In some specific examples, the transactivation domain comprises the Glucocorticoid Receptor Tau1 (Minimal) domain, which is abbreviated to “GR1” in the present disclosure.

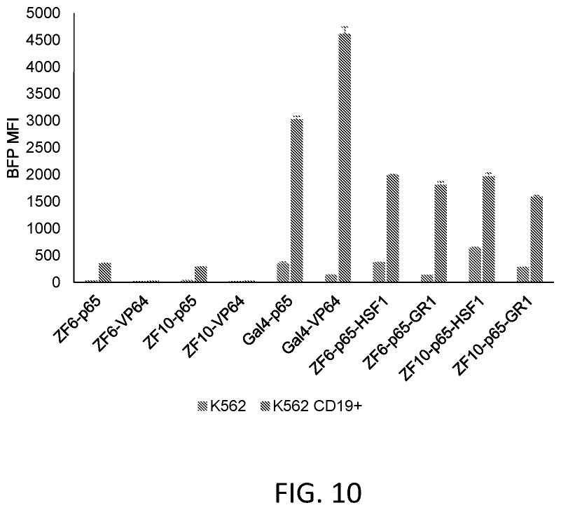

Thus, in some embodiments, the effector domain of the ZTE includes a fusion protein comprising p65 activation domain of NFκB and a sequence derived from HSF1. In other embodiments, the effector domain of the ZTE includes a fusion protein comprising p65 activation domain of NFκB and a sequence derived from GR1. Additional sequences suitable for the compositions and methods disclosed herein include, but are not limited to, sequences derived from the HIF1a, ATF6, ELF3, p53, ZNF473, FOXO, and Myb. Some exemplary constructs are provided herein, for example, in SEQ ID NOS: 130-133, 136, 137, 138, 139, 141, 142, 144, and 145. In some embodiments, these fusion TAD domains (e.g., the p65-HSF1 or the p65-GR1 TADs) produce more effective transcriptional activation of a gene of interest than TAD domains without the fusion of the additional sequence (e.g., the p65 TAD alone). See, for example, the results described in Example 1 and FIG. 10.

In some embodiments, the effector domain of the ZTE includes a transcription repressor domain. Non-limiting examples of transcription repressor domains suitable for use in the compositions and methods disclosed herein include a Kruppel associated box repression domain (KRAB); a Repressor Element Silencing Transcription Factor repression domain (REST); a WRPW motif of the hairy-related basic helix-loop-helix repressor proteins repression domain (WRPW); a DNA (cytosine-5)-methyltransferase 3B repression domain (DNMT3B); and an HP1 alpha chromoshadow repression domain. In some embodiments, the transcription repressor domain includes a KRAB repressor domain.

Accordingly, in some embodiments, the effector domain of the ZTE includes an epigenetic effector domain. Examples of epigenetic effector domain suitable for use in the compositions and methods disclosed herein include, but are not limited to, DNA methyltransferases DNMT (DNMT1, DNMT3), HAT1, GCN5, PCAF, MLL, SET, DOT1, SUV39H, G9a, KAT2A/B, EZH1/2, TET1/2, SIRT family protein effector domains, histone deacetylases, LSD1, and KDM family protein effector domains.

Effectors domains suitable for the compositions and methods of the disclosure can be naturally-occurring transcriptional regulators or can be engineered, designed, or modified so as to provide desired and/or improved properties, e.g., modulating transcription a eukaryotic cell. In some embodiments, the effector domain is derived from an animal protein. In some embodiments, the effector domain is derived from a mammalian protein. In some embodiments, the effector domain is derived from non-human primate protein. In some embodiments, the effector domain is derived from a human protein.

In some embodiments, the ICD of the chimeric receptors disclosed herein further includes a nuclear transport signal sequence (NLS). Nuclear localization signals (NLSs) are short peptide motifs that mediate the nuclear import of proteins by binding to their receptors, known as importins (karyopherins).

It will be understood by one having ordinary skill in the art that a transcriptional effector can be a transcriptional activator or a transcriptional repressor. In some embodiments, the transcriptional effector is a transcriptional repressor. In some embodiments, the transcriptional effector is a transcriptional activator. In some embodiments, the transcriptional effector directly regulates differentiation of the cell. In some embodiments, the transcriptional effector indirectly modulates (e.g., regulates) differentiation of the cell by modulating the expression of a second transcription factor.

Chimeric polypeptides and synZTE-containing Notch receptors of the present disclosure can be chimeric polypeptides of any length, including chimeric polypeptides that are generally between about 100 amino acids (aa) to about 1000 aa, e.g., from about 100 aa to about 200 aa, from about 150 aa to about 250 aa, from about 200 aa to about 300 aa, from about 250 aa to about 350 aa, from about 300 aa to about 400 aa, from about 350 aa to about 450 aa, from about 400 aa to about 500 aa in length. In some embodiments, the disclosed chimeric polypeptides are at least about 100, 200, 300, 400, 500, 600, 700, 750, 800, 850, 900, 950, or 1,000 aa in length. In some embodiments, the disclosed chimeric polypeptides are less than about 1,500, 1,400, 1,300, 1,200, 1,100, 1,000, 950, 900, 850, 800, 750, 700, 600, 500, 400, 350, 300, 250, or 200 aa in length. In some embodiments, the disclosed chimeric polypeptides are generally between about 400 aa to about 450 aa, from about 450 aa to about 500 aa, from about 500 aa to about 550 aa, from about 550 aa to about 600 aa, from about 600 aa to about 650 aa, from about 650 aa to about 700 aa, from about 700 aa to about 750 aa, from about 750 aa to about 800 aa, from about 800 aa to about 850 aa, from about 850 aa to about 900 aa, from about 900 aa to about 950 aa, or from about 950 aa to about 1000 aa in length. In some cases, the chimeric polypeptides of the present disclosure have a length of about 300 aa to about 400 aa. In some cases, the chimeric polypeptides of the present disclosure have a length of about 300 aa to about 350 aa. In some cases, the chimeric polypeptides of the present disclosure have a length of about 300 aa to about 325 aa. In some cases, the chimeric polypeptides of the present disclosure have a length of about 350 aa to about 400 aa. In some cases, the chimeric polypeptides of the present disclosure have a length of 750 aa to 850 aa.

Additional Domains

In some embodiments, the Notch extracellular domains located N-terminally to the TMD can further include an additional domain, for example a membrane localization signal such as a CD8A signal, a detectable marker such as a myc tag or his tag, and the like. Without being bound to any particular theory, it may be beneficial to incorporate additional domains N-terminally to the hinge domain. This is because, incorporating bulky features (such as an NRR) adjacent to the TMD would affect receptor activity, unless it is spaced far enough away. It is also contemplated that the chimeric polypeptides and synZTE-containing Notch receptors as described herein can be further engineered to include one or more additional features such as, a signal sequence, a detectable label, a tumor-specific cleavage site, a disease-specific cleavage site, or combinations thereof. For example, several proteases (such as matrix metalloproteases) are upregulated in cancers, allowing tumor-specific cleavage specificity not via a specific cleavage site but via higher levels of specific proteases. Additional information in this regard can be found in, for example, J. S. Dudani et al., Annu. Rev. Cancer Biol. (2018), 2:353-76, which is herein incorporated by reference.

In some embodiments, the synZTE-containing Notch receptor of the disclosure includes: (a) an ECD including an amino acid sequence having at least 80% sequence identity to SEQ ID NO: 10; (b) a linking polypeptide including an amino acid sequence having at least 80% sequence identity to SEQ ID NO: 11; (c) a TMD including an amino acid sequence having at least 80% sequence identity to SEQ ID NO: 31; (c) a stop-transfer-sequence domain including an amino acid sequence having at least 80% sequence identity to any one of SEQ ID NOS: 39-40; and (d) a ZTE including an amino acid sequence having at least 80% sequence identity to SEQ ID NO: 56.

In some embodiments, the synZTE-containing Notch receptor of the disclosure includes: (a) an ECD including an amino acid sequence having at least 90% sequence identity to SEQ ID NO: 10; (b) a linking polypeptide including an amino acid sequence having at least 90% sequence identity to SEQ ID NO: 11; (c) a TMD including an amino acid sequence having at least 90% sequence identity to SEQ ID NO: 31; (c) a stop-transfer-sequence domain including an amino acid sequence having at least 90% sequence identity to any one of SEQ ID NOS: 39-40; and (d) a ZTE including an amino acid sequence having at least 90% sequence identity to SEQ ID NO: 56.

In some embodiments, the synZTE-containing Notch receptor of the disclosure includes: (a) an ECD including an amino acid sequence having at least 95% sequence identity to SEQ ID NO: 10; (b) a linking polypeptide including an amino acid sequence having at least 95% sequence identity to SEQ ID NO: 11; (c) a TMD including an amino acid sequence having at least 95% sequence identity to SEQ ID NO: 31; (c) a stop-transfer-sequence domain including an amino acid sequence having at least 95% sequence identity to any one of SEQ ID NOS: 39-40; and (d) a ZTE including an amino acid sequence having at least 95% sequence identity to SEQ ID NO: 56.

In some embodiments, the synZTE-containing Notch receptor of the disclosure includes: (a) an ECD including an amino acid sequence having at least 80% sequence identity to SEQ ID NO: 10; (b) a hinge domain including an amino acid sequence having at least 80% sequence identity to SEQ ID NO: 21; (c) a TMD including an amino acid sequence having at least 80% sequence identity to SEQ ID NO: 31; (c) a stop-transfer-sequence domain including an amino acid sequence having at least 80% sequence identity to any one of SEQ ID NOS: 39-40; and (d) a ZTE including an amino acid sequence having at least 80% sequence identity to any one of SEQ ID NOS: 55-60.

In some embodiments, the synZTE-containing Notch receptor of the disclosure includes: (a) an ECD including an amino acid sequence having at least 90% sequence identity to SEQ ID NO: 10; (b) a hinge domain including an amino acid sequence having at least 90% sequence identity to SEQ ID NO: 21; (c) a TMD including an amino acid sequence having at least 90% sequence identity to SEQ ID NO: 31; (c) a stop-transfer-sequence domain including an amino acid sequence having at least 90% sequence identity to any one of SEQ ID NOS: 39-40; and (d) a ZTE including an amino acid sequence having at least 90% sequence identity to any one of SEQ ID NOS: 55-60.

In some embodiments, the synZTE-containing Notch receptor of the disclosure includes: (a) an ECD including an amino acid sequence having at least 95% sequence identity to SEQ ID NO: 10; (b) a hinge domain including an amino acid sequence having at least 95% sequence identity to SEQ ID NO: 21; (c) a TMD including an amino acid sequence having at least 95% sequence identity to SEQ ID NO: 31; (c) a stop-transfer-sequence domain including an amino acid sequence having at least 95% sequence identity to any one of SEQ ID NOS: 39-40; and (d) a ZTE including an amino acid sequence having at least 95% sequence identity to any one of SEQ ID NOS: 55-60.

In some embodiments, the synZTE-containing Notch receptor of the disclosure includes an amino acid sequence having at least about 80%, 90%, 95%, 96%, 97, 98%, 99%, or 100% sequence identity to a chimeric receptor disclosed herein. In some embodiments, provided herein are synZTE-containing Notch receptors including an amino acid sequence having at least about 80%, 90%, 95%, 96%, 97, 98%, 99%, or 100% sequence identity to any one of SEQ ID NOS: 1-9 and 113-123 identified in the Sequence Listing. In some embodiments, the chimeric polypeptide includes an amino acid sequence having at least about 80%, 90%, 95%, 96%, 97, 98%, 99%, or 100% sequence identity to SEQ ID NO: 1. In some embodiments, the synZTE-containing Notch receptor includes an amino acid sequence having at least about 80%, 90%, 95%, 96%, 97, 98%, 99%, or 100% sequence identity to SEQ ID NO: 2. In some embodiments, the synZTE-containing Notch receptor includes an amino acid sequence having at least about 80%, 90%, 95%, 96%, 97, 98%, 99%, or 100% sequence identity to SEQ ID NO: 3. In some embodiments, the synZTE-containing Notch receptor includes an amino acid sequence having at least about 80%, 90%, 95%, 96%, 97, 98%, 99%, or 100% sequence identity to SEQ ID NO: 4. In some embodiments, the synZTE-containing Notch receptor includes an amino acid sequence having at least about 80%, 90%, 95%, 96%, 97, 98%, 99%, or 100% sequence identity to SEQ ID NO: 5. In some embodiments, the synZTE-containing Notch receptor includes an amino acid sequence having at least about 80%, 90%, 95%, 96%, 97, 98%, 99%, or 100% sequence identity to SEQ ID NO: 6. In some embodiments, the synZTE-containing Notch receptor includes an amino acid sequence having at least about 80%, 90%, 95%, 96%, 97, 98%, 99%, or 100% sequence identity to SEQ ID NO: 7. In some embodiments, the synZTE-containing Notch receptor includes an amino acid sequence having at least about 80%, 90%, 95%, 96%, 97, 98%, 99%, or 100% sequence identity to SEQ ID NO: 8. In some embodiments, the synZTE-containing Notch receptor includes an amino acid sequence having at least about 80%, 90%, 95%, 96%, 97, 98%, 99%, or 100% sequence identity to SEQ ID NO: 9.

In some embodiments, the chimeric polypeptide includes an amino acid sequence having at least about 80%, 90%, 95%, 96%, 97, 98%, 99%, or 100% sequence identity to SEQ ID NO: 113. In some embodiments, the synZTE-containing Notch receptor includes an amino acid sequence having at least about 80%, 90%, 95%, 96%, 97, 98%, 99%, or 100% sequence identity to SEQ ID NO: 114. In some embodiments, the synZTE-containing Notch receptor includes an amino acid sequence having at least about 80%, 90%, 95%, 96%, 97, 98%, 99%, or 100% sequence identity to SEQ ID NO: 115. In some embodiments, the synZTE-containing Notch receptor includes an amino acid sequence having at least about 80%, 90%, 95%, 96%, 97, 98%, 99%, or 100% sequence identity to SEQ ID NO: 116. In some embodiments, the synZTE-containing Notch receptor includes an amino acid sequence having at least about 80%, 90%, 95%, 96%, 97, 98%, 99%, or 100% sequence identity to SEQ ID NO: 117. In some embodiments, the synZTE-containing Notch receptor includes an amino acid sequence having at least about 80%, 90%, 95%, 96%, 97, 98%, 99%, or 100% sequence identity to SEQ ID NO: 118. In some embodiments, the synZTE-containing Notch receptor includes an amino acid sequence having at least about 80%, 90%, 95%, 96%, 97, 98%, 99%, or 100% sequence identity to SEQ ID NO: 119. In some embodiments, the synZTE-containing Notch receptor includes an amino acid sequence having at least about 80%, 90%, 95%, 96%, 97, 98%, 99%, or 100% sequence identity to SEQ ID NO: 120. In some embodiments, the synZTE-containing Notch receptor includes an amino acid sequence having at least about 80%, 90%, 95%, 96%, 97, 98%, 99%, or 100% sequence identity to SEQ ID NO: 121. In some embodiments, the synZTE-containing Notch receptor includes an amino acid sequence having at least about 80%, 90%, 95%, 96%, 97, 98%, 99%, or 100% sequence identity to SEQ ID NO: 122. In some embodiments, the synZTE-containing Notch receptor includes an amino acid sequence having at least about 80%, 90%, 95%, 96%, 97, 98%, 99%, or 100% sequence identity to SEQ ID NO: 123. In some embodiments, the synZTE-containing Notch receptor includes an amino acid sequence having at least about 80%, 90%, 95%, 96%, 97, 98%, 99%, or 100% sequence identity to SEQ ID NO: 124. In some embodiments, the synZTE-containing Notch receptor includes an amino acid sequence having at least about 80%, 90%, 95%, 96%, 97, 98%, 99%, or 100% sequence identity to SEQ ID NO: 125. In some embodiments, the synZTE-containing Notch receptor includes an amino acid sequence having at least about 80%, 90%, 95%, 96%, 97, 98%, 99%, or 100% sequence identity to SEQ ID NO: 126. In some embodiments, the synZTE-containing Notch receptor includes an amino acid sequence having at least about 80%, 90%, 95%, 96%, 97, 98%, 99%, or 100% sequence identity to SEQ ID NO: 127. In some embodiments, the synZTE-containing Notch receptor includes an amino acid sequence having at least about 80%, 90%, 95%, 96%, 97, 98%, 99%, or 100% sequence identity to SEQ ID NO: 128. In some embodiments, the synZTE-containing Notch receptor includes an amino acid sequence having at least about 80%, 90%, 95%, 96%, 97, 98%, 99%, or 100% sequence identity to SEQ ID NO: 129. In some embodiments, the synZTE-containing Notch receptor includes an amino acid sequence having at least about 80%, 90%, 95%, 96%, 97, 98%, 99%, or 100% sequence identity to SEQ ID NO: 130. In some embodiments, the synZTE-containing Notch receptor includes an amino acid sequence having at least about 80%, 90%, 95%, 96%, 97, 98%, 99%, or 100% sequence identity to SEQ ID NO: 131. In some embodiments, the synZTE-containing Notch receptor includes an amino acid sequence having at least about 80%, 90%, 95%, 96%, 97, 98%, 99%, or 100% sequence identity to SEQ ID NO: 132. In some embodiments, the synZTE-containing Notch receptor includes an amino acid sequence having at least about 80%, 90%, 95%, 96%, 97, 98%, 99%, or 100% sequence identity to SEQ ID NO: 133. In some embodiments, the synZTE-containing Notch receptor includes an amino acid sequence having at least about 80%, 90%, 95%, 96%, 97, 98%, 99%, or 100% sequence identity to SEQ ID NO: 134. In some embodiments, the synZTE-containing Notch receptor includes an amino acid sequence having at least about 80%, 90%, 95%, 96%, 97, 98%, 99%, or 100% sequence identity to SEQ ID NO: 135. In some embodiments, the synZTE-containing Notch receptor includes an amino acid sequence having at least about 80%, 90%, 95%, 96%, 97, 98%, 99%, or 100% sequence identity to SEQ ID NO: 136. In some embodiments, the synZTE-containing Notch receptor includes an amino acid sequence having at least about 80%, 90%, 95%, 96%, 97, 98%, 99%, or 100% sequence identity to SEQ ID NO: 137. In some embodiments, the synZTE-containing Notch receptor includes an amino acid sequence having at least about 80%, 90%, 95%, 96%, 97, 98%, 99%, or 100% sequence identity to SEQ ID NO: 138. In some embodiments, the synZTE-containing Notch receptor includes an amino acid sequence having at least about 80%, 90%, 95%, 96%, 97, 98%, 99%, or 100% sequence identity to SEQ ID NO: 139. In some embodiments, the synZTE-containing Notch receptor includes an amino acid sequence having at least about 80%, 90%, 95%, 96%, 97, 98%, 99%, or 100% sequence identity to SEQ ID NO: 140. In some embodiments, the synZTE-containing Notch receptor includes an amino acid sequence having at least about 80%, 90%, 95%, 96%, 97, 98%, 99%, or 100% sequence identity to SEQ ID NO: 141. In some embodiments, the synZTE-containing Notch receptor includes an amino acid sequence having at least about 80%, 90%, 95%, 96%, 97, 98%, 99%, or 100% sequence identity to SEQ ID NO: 142. In some embodiments, the synZTE-containing Notch receptor includes an amino acid sequence having at least about 80%, 90%, 95%, 96%, 97, 98%, 99%, or 100% sequence identity to SEQ ID NO: 143. In some embodiments, the synZTE-containing Notch receptor includes an amino acid sequence having at least about 80%, 90%, 95%, 96%, 97, 98%, 99%, or 100% sequence identity to SEQ ID NO: 144. In some embodiments, the synZTE-containing Notch receptor includes an amino acid sequence having at least about 80%, 90%, 95%, 96%, 97, 98%, 99%, or 100% sequence identity to SEQ ID NO: 145.

### Nucleic Acid Molecules

In another aspect, provided herein are various nucleic acid molecules including nucleotide sequences encoding the chimeric polypeptides and synZTE-containing Notch receptors of the disclosure, including expression cassettes, and expression vectors containing these nucleic acid molecules operably linked to heterologous nucleic acid sequences such as, for example, regulatory sequences which facilitate in vivo expression of the receptor in a host cell.

Nucleic acid molecules of the present disclosure can be of any length, including for example, between about 1.5 Kb and about 50 Kb, between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, provided herein is a nucleic acid molecule including a nucleotide sequence encoding a chimeric polypeptide or synZTE-containing Notch receptor including, from N-terminus to C-terminus: (a) an extracellular ligand-binding domain having a binding affinity for a selected ligand; (b) a linking polypeptide having: (i) at least about 80%, 85%, 90%, 95%, 96%, 97%, 98%, 99%, or 100% sequence identity to a Notch juxtamembrane domain (JMD) wherein a LIN-12-Notch repeat (LNR) and/or a heterodimerization domain (HD) of a Notch receptor has been deleted; (ii) at least about 80%, 85%, 90%, 95%, 96%, 97%, 98%, 99%, or 100% sequence identity to a polypeptide hinge domain; or (iii) a sequence of about 2 to about 40 amino acid residues; (c) a transmembrane domain having at least about 80%, 85%, 90%, 95%, 96%, 97%, 98%, 99%, or 100% sequence identity to the transmembrane domain of a Type 1 transmembrane receptor and including one or more ligand-inducible proteolytic cleavage sites; and (d) an intracellular domain including a zinc finger-containing transcriptional effector (ZTE), wherein binding of the selected ligand to the extracellular binding domain induces cleavage at a ligand-inducible proteolytic cleavage site within the transmembrane domain.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the chimeric polypeptide or synZTE-containing Notch receptor as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that generally facilitate transfer of the nucleic acid molecule or integration into the genome of a cell or to a viral particle that mediates nucleic acid transfer. Viral particles will generally include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

In some embodiments, provided herein are nucleic acid molecules encoding a polypeptide with an amino acid sequence having at least about 80%, 90%, 95%, 96%, 97, 98%, 99%, or 100% sequence identity to a chimeric receptor disclosed herein. In some embodiments, provided herein are nucleic acid molecules encoding a polypeptide with an amino acid sequence having at least about 80%, 90%, 95%, 96%, 97, 98%, 99%, or 100% sequence identity to any one of SEQ ID NOS: 1-9 and 113-123 identified in the Sequence Listing. In some embodiments, the nucleic acid molecules encode a polypeptide with an amino acid sequence having at least about 80%, 90%, 95%, 96%, 97, 98%, 99%, or 100% sequence identity to SEQ ID NO: 1. In some embodiments, the nucleic acid molecules encode a polypeptide with an amino acid sequence having at least about 80%, 90%, 95%, 96%, 97, 98%, 99%, or 100% sequence identity to SEQ ID NO: 2. In some embodiments, the nucleic acid molecules encode a polypeptide with an amino acid sequence having at least about 80%, 90%, 95%, 96%, 97, 98%, 99%, or 100% sequence identity to SEQ ID NO: 3. In some embodiments, the nucleic acid molecules encode a polypeptide with an amino acid sequence having at least about 80%, 90%, 95%, 96%, 97, 98%, 99%, or 100% sequence identity to SEQ ID NO: 4. In some embodiments, the nucleic acid molecules encode a polypeptide with an amino acid sequence having at least about 80%, 90%, 95%, 96%, 97, 98%, 99%, or 100% sequence identity to SEQ ID NO: 5. In some embodiments, the nucleic acid molecules encode a polypeptide with an amino acid sequence having at least about 80%, 90%, 95%, 96%, 97, 98%, 99%, or 100% sequence identity to SEQ ID NO: 6. In some embodiments, the nucleic acid molecules encode a polypeptide with an amino acid sequence having at least about 80%, 90%, 95%, 96%, 97, 98%, 99%, or 100% sequence identity to SEQ ID NO: 7. In some embodiments, the nucleic acid molecules encode a polypeptide with an amino acid sequence having at least about 80%, 90%, 95%, 96%, 97, 98%, 99%, or 100% sequence identity to SEQ ID NO: 8. In some embodiments, the nucleic acid molecules encode a polypeptide with an amino acid sequence having at least about 80%, 90%, 95%, 96%, 97, 98%, 99%, or 100% sequence identity to SEQ ID NO: 9.

In some embodiments, the nucleic acid molecules encode a polypeptide with an amino acid sequence having at least about 80%, 90%, 95%, 96%, 97, 98%, 99%, or 100% sequence identity to SEQ ID NO: 113. In some embodiments, the nucleic acid molecules encode a polypeptide with an amino acid sequence having at least about 80%, 90%, 95%, 96%, 97, 98%, 99%, or 100% sequence identity to SEQ ID NO: 114. In some embodiments, the nucleic acid molecules encode a polypeptide with an amino acid sequence having at least about 80%, 90%, 95%, 96%, 97, 98%, 99%, or 100% sequence identity to SEQ ID NO: 115. In some embodiments, the nucleic acid molecules encode a polypeptide with an amino acid sequence having at least about 80%, 90%, 95%, 96%, 97, 98%, 99%, or 100% sequence identity to SEQ ID NO: 116. In some embodiments, the nucleic acid molecules encode a polypeptide with an amino acid sequence having at least about 80%, 90%, 95%, 96%, 97, 98%, 99%, or 100% sequence identity to SEQ ID NO: 117. In some embodiments, the nucleic acid molecules encode a polypeptide with an amino acid sequence having at least about 80%, 90%, 95%, 96%, 97, 98%, 99%, or 100% sequence identity to SEQ ID NO: 118. In some embodiments, the nucleic acid molecules encode a polypeptide with an amino acid sequence having at least about 80%, 90%, 95%, 96%, 97, 98%, 99%, or 100% sequence identity to SEQ ID NO: 119. In some embodiments, the nucleic acid molecules encode a polypeptide with an amino acid sequence having at least about 80%, 90%, 95%, 96%, 97, 98%, 99%, or 100% sequence identity to SEQ ID NO: 120. In some embodiments, the nucleic acid molecules encode a polypeptide with an amino acid sequence having at least about 80%, 90%, 95%, 96%, 97, 98%, 99%, or 100% sequence identity to SEQ ID NO: 121. In some embodiments, the nucleic acid molecules encode a polypeptide with an amino acid sequence having at least about 80%, 90%, 95%, 96%, 97, 98%, 99%, or 100% sequence identity to SEQ ID NO: 122. In some embodiments, the nucleic acid molecules encode a polypeptide with an amino acid sequence having at least about 80%, 90%, 95%, 96%, 97, 98%, 99%, or 100% sequence identity to SEQ ID NO: 123. In some embodiments, the nucleic acid molecules encode a polypeptide with an amino acid sequence having at least about 80%, 90%, 95%, 96%, 97, 98%, 99%, or 100% sequence identity to SEQ ID NO: 124. In some embodiments, the nucleic acid molecules encode a polypeptide with an amino acid sequence having at least about 80%, 90%, 95%, 96%, 97, 98%, 99%, or 100% sequence identity to SEQ ID NO: 125. In some embodiments, the nucleic acid molecules encode a polypeptide with an amino acid sequence having at least about 80%, 90%, 95%, 96%, 97, 98%, 99%, or 100% sequence identity to SEQ ID NO: 126. In some embodiments, the nucleic acid molecules encode a polypeptide with an amino acid sequence having at least about 80%, 90%, 95%, 96%, 97, 98%, 99%, or 100% sequence identity to SEQ ID NO: 127. In some embodiments, the nucleic acid molecules encode a polypeptide with an amino acid sequence having at least about 80%, 90%, 95%, 96%, 97, 98%, 99%, or 100% sequence identity to SEQ ID NO: 128. In some embodiments, the nucleic acid molecules encode a polypeptide with an amino acid sequence having at least about 80%, 90%, 95%, 96%, 97, 98%, 99%, or 100% sequence identity to SEQ ID NO: 129. In some embodiments, the nucleic acid molecules encode a polypeptide with an amino acid sequence having at least about 80%, 90%, 95%, 96%, 97, 98%, 99%, or 100% sequence identity to SEQ ID NO: 130. In some embodiments, the nucleic acid molecules encode a polypeptide with an amino acid sequence having at least about 80%, 90%, 95%, 96%, 97, 98%, 99%, or 100% sequence identity to SEQ ID NO: 131. In some embodiments, the nucleic acid molecules encode a polypeptide with an amino acid sequence having at least about 80%, 90%, 95%, 96%, 97, 98%, 99%, or 100% sequence identity to SEQ ID NO: 132. In some embodiments, the nucleic acid molecules encode a polypeptide with an amino acid sequence having at least about 80%, 90%, 95%, 96%, 97, 98%, 99%, or 100% sequence identity to SEQ ID NO: 133. In some embodiments, the nucleic acid molecules encode a polypeptide with an amino acid sequence having at least about 80%, 90%, 95%, 96%, 97, 98%, 99%, or 100% sequence identity to SEQ ID NO: 134. In some embodiments, the nucleic acid molecules encode a polypeptide with an amino acid sequence having at least about 80%, 90%, 95%, 96%, 97, 98%, 99%, or 100% sequence identity to SEQ ID NO: 135. In some embodiments, the nucleic acid molecules encode a polypeptide with an amino acid sequence having at least about 80%, 90%, 95%, 96%, 97, 98%, 99%, or 100% sequence identity to SEQ ID NO: 136. In some embodiments, the nucleic acid molecules encode a polypeptide with an amino acid sequence having at least about 80%, 90%, 95%, 96%, 97, 98%, 99%, or 100% sequence identity to SEQ ID NO: 137. In some embodiments, the nucleic acid molecules encode a polypeptide with an amino acid sequence having at least about 80%, 90%, 95%, 96%, 97, 98%, 99%, or 100% sequence identity to SEQ ID NO: 138. In some embodiments, the nucleic acid molecules encode a polypeptide with an amino acid sequence having at least about 80%, 90%, 95%, 96%, 97, 98%, 99%, or 100% sequence identity to SEQ ID NO: 139. In some embodiments, the nucleic acid molecules encode a polypeptide with an amino acid sequence having at least about 80%, 90%, 95%, 96%, 97, 98%, 99%, or 100% sequence identity to SEQ ID NO: 140. In some embodiments, the nucleic acid molecules encode a polypeptide with an amino acid sequence having at least about 80%, 90%, 95%, 96%, 97, 98%, 99%, or 100% sequence identity to SEQ ID NO: 141. In some embodiments, the nucleic acid molecules encode a polypeptide with an amino acid sequence having at least about 80%, 90%, 95%, 96%, 97, 98%, 99%, or 100% sequence identity to SEQ ID NO: 142. In some embodiments, the nucleic acid molecules encode a polypeptide with an amino acid sequence having at least about 80%, 90%, 95%, 96%, 97, 98%, 99%, or 100% sequence identity to SEQ ID NO: 143. In some embodiments, the nucleic acid molecules encode a polypeptide with an amino acid sequence having at least about 80%, 90%, 95%, 96%, 97, 98%, 99%, or 100% sequence identity to SEQ ID NO: 144. In some embodiments, the nucleic acid molecules encode a polypeptide with an amino acid sequence having at least about 80%, 90%, 95%, 96%, 97, 98%, 99%, or 100% sequence identity to SEQ ID NO: 145.

In some embodiments, the nucleic acid molecules disclosed herein further include a response element, wherein the response element includes: (a) a ZFA target sequence; (b) an engineered responsive promoter operably linked to the ZF target sequence; and (c) a polynucleotide of interest. In some embodiments, the polynucleotide of interest encodes a regulatory RNA, a regulatory protein, a therapeutic protein, or a detectable label.

Engineered responsive promoter are designed by placing instances of the targetable DNA sequences (e.g., ZF binding sites) upstream of constitutive promoters. The targetable DNA sequences are operably linked to the promoters such that the occupancy of synTFs on the targetable DNA sequences regulates the activity of the promoter in gene expression. The combination of synTFs and a targetable DNA sequence-promoter forms a unique expression system that is artificial, scalable, and regulatable, for the expression of desired genes placed within the expression systems, with no or minimal effects on the expression of endogenous genes, meaning no or minimal off-site gene regulation of endogenous genes.

In some embodiments of engineered responsive promoter described, the promoter described herein can be a full-length functional promoter or a minimal promoter having very limited or no transcription initiation therefrom absent the assistance of added transcription factors. Non-limiting examples of full-length functional promoters include CMV, UBCbc, EF1 alpha, SV40, PGK, CAG, beta actin, U6 and H1. Non-limiting examples of minimal promoters include minimal CMV, and minimal TK and any synthetically designed promoters composed of core minimal promoter elements and regulating enhancer elements (e.g., HSE, TRE, NFAT/AP-1 binding elements).

The polynucleotide of interest can be a regulatory or signaling nucleic acid, or can encode any protein that can be expressed by the engineered cell. In some embodiments, the protein is a detectable label. In some embodiments, the detectable label is a fluorescent protein or a chromogenic protein. Suitable fluorescent proteins include GFP, mCherry, mTomato, mStrawberry, and other. In some embodiments, the protein is a therapeutic protein. In some embodiments, the therapeutic protein is a chimeric antigen receptor (CAR). In some embodiments, the therapeutic protein is a therapeutic antibody. In some embodiments, the therapeutic antibody is an antibody capable of specifically binding to an immune checkpoint receptor, such as CTLA-4, PD-1, PD-L1, or others. In some embodiments, the protein is a cytokine. In some embodiments, the cytokine is IL-12 or IFNγ. In some embodiments, the polynucleotide of interest encodes a regulatory nucleic acid. In some embodiments, the regulatory nucleic acid is an RNA. In some embodiments, the regulatory RNA is an siRNA, shRNA, or miRNA.

The nucleic acid sequences encoding the chimeric receptors can be optimized for expression in the host cell of interest. For example, the G-C content of the sequence can be adjusted to average levels for a given cellular host, as calculated by reference to known genes expressed in the host cell. Methods for codon usage optimization are known in the art. Codon usages within the coding sequence of the chimeric receptor disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the chimeric receptors disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any chimeric receptor or synZTE-containing Notch receptor disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

DNA vectors can be introduced into eukaryotic cells via conventional transformation or transfection techniques. Suitable methods for transforming or transfecting cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.). For example, a chimeric receptor as disclosed herein can be produced in a eukaryotic cell, such as a mammalian cell (e.g., COS cells, NIH 3T3 cells, or HeLa cells). These cells are available from many sources, including the American Type Culture Collection (Manassas, Va.). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a chimeric receptor) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

### Recombinant Cells and Cell Cultures

The nucleic acid of the present disclosure can be introduced into a host cell, such as, for example, a human T lymphocyte, to produce a recombinant cell containing the nucleic acid molecule. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, adeno-associated virus (AAV) is engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell (T_{H}), a cytotoxic T cell (T_{CTL}), or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+T cytotoxic lymphocyte cell selected from the group consisting of naïve CD8+ T cells, central memory CD8+ T cells, effector memory CD8+ T cells, and bulk CD8+ T cells. In some embodiments of the cell, the cell is a CD4+T helper lymphocyte cell selected from the group consisting of naïve CD4+ T cells, central memory CD4+ T cells, effector memory CD4+ T cells, and bulk CD4+ T cells. In some embodiments, the cell can be obtained by leukapheresis performed on a sample obtained from a subject. In some embodiments, the subject is a human subject. In some embodiments, the human subject is a patient.

In some embodiments, the recombinant cell further includes the recombinant cell further includes an engineered response element including i) a ZFA target sequence to which a ZFA of the ZTE of the chimeric polypeptide specifically binds, ii) a promoter sequence, wherein the nucleic acid target sequence is operably linked to the 5′ end of the promoter sequence, and iii) a polynucleotide of interest operably linked to the promoter sequence, wherein binding of the ZTE to the ZFA target sequence modulates transcription initiation of a polynucleotide of interest.

In some embodiments, the ZFA target sequence of the engineered response element includes a sequence that is orthogonal to the recombinant cell genome. In some embodiments, the ZFA target sequence includes a nucleotide sequence selected from the group consisting of SEQ ID NOS: 61-71.

In some embodiments, the engineered response element is present in a nucleic acid vector, plasmid, DNA minicircle, minichromosome, or host chromosome. In some embodiments, the engineered response element is incorporated into the same nucleic acid molecule that encodes a chimeric polypeptide or synZTE-containing Notch receptor of the disclosure. In some embodiments, the engineered response element is incorporated into a second expression vector that is separate from the nucleic acid molecule encoding the chimeric polypeptide or synZTE-containing Notch receptor of the disclosure. In some embodiments, the polynucleotide of interest encodes a protein, regulatory RNA, or an antisense oligonucleotide. In some embodiments, the protein is heterologous to the recombinant cell. A heterologous protein is one that is not normally found in the cell, e.g., not normally produced by the cell. Exemplary types of proteins suitable for use with the compositions and methods disclosed herein include cytokines, cytotoxins, chemokines, immunomodulators, pro-apoptotic factors, anti-apoptotic factors, hormones, immune cell receptors, differentiation factors, dedifferentiation factors, or reporters. In some embodiments, the immune cell receptor is a T-cell receptor (TCR). In some embodiments, the immune cell receptor is a chimeric antigen receptor (CAR).

In another aspect, some embodiments of the disclosure relate to methods for making a recombinant cell, including (a) providing a cell capable of protein expression and (b) contacting the provided cell with a recombinant nucleic acid of the disclosure. In some embodiments, the method for making a recombinant cell further includes (c) transducing the cell with a recombinant nucleic acid that encodes a response element, wherein the response element includes: (i) a ZFA target sequence; (ii) an engineered responsive promoter operably linked to the ZF target sequence; and (iii) a polynucleotide of interest.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Pharmaceutical Compositions

In some embodiments, the chimeric polypeptides, synZTE-containing Notch receptors, nucleic acids, and recombinant cells of the disclosure can be incorporated into compositions, including pharmaceutical compositions. Such compositions generally include one or more of the nucleic acids of the disclosure, and/or recombinant cells of the disclosure, and a pharmaceutically acceptable excipient, e.g., carrier.

In some embodiments, the composition includes a recombinant nucleic acid as disclosed herein and a pharmaceutically acceptable excipient. In some embodiments, the recombinant nucleic acid is encapsulated in a viral capsid or a lipid nanoparticle.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

### Methods of the Disclosure

Administration of any one of the therapeutic compositions described herein, e.g., nucleic acids, recombinant cells, and pharmaceutical compositions, can be used to treat subjects for relevant health conditions or diseases, such as cancers and chronic infections. In some embodiments, the nucleic acids, recombinant cells, and pharmaceutical compositions described herein can be incorporated into therapeutic agents for use in methods of treating an individual who has, who is suspected of having, or who may be at high risk for developing one or more autoimmune disorders or diseases associated with checkpoint inhibition. Exemplary autoimmune disorders and diseases can include, without limitation, celiac disease, type 1 diabetes, Graves' disease, inflammatory bowel disease, multiple sclerosis, psoriasis, rheumatoid arthritis, and systemic lupus erythematosus.

Accordingly, in one aspect, some embodiments of the disclosure relate to methods for modulating (e.g., inhibiting) an activity of a target cell in an individual, the methods include administering to the individual a first therapy including one or more of nucleic acids, recombinant cells, and pharmaceutical compositions as disclosed herein, wherein the first therapy modulates (e.g., inhibits) an activity of the target cell. For example, the target cell may be inhibited if its proliferation is reduced, if its pathologic or pathogenic behavior is reduced, if it is destroyed or killed, etc. Inhibition includes a reduction of the measured pathologic or pathogenic behavior of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cells disclosed herein, wherein the recombinant cells inhibit an activity of the target cells in the individual. Generally, the target cells of the disclosed methods can be any cell type in an individual and can be, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell. In some embodiments, the target cell is a cancer cell. In some embodiments, the modulation of the activity the target cell results in the death of the target cell.

In another aspect, some embodiments of the disclosure relate to methods for the treatment of a health condition (e.g., disease) in an individual in need thereof, the methods include administering to the individual a first therapy including one or more of the recombinant cells including a chimeric polypeptide or synZTE-containing Notch receptor as disclosed herein, and/or pharmaceutical compositions as disclosed herein, wherein the first therapy treats the disease in the individual. In some embodiments, the methods include administering to the individual a first therapy including an effective number of the recombinant cells as disclosed herein, wherein the recombinant cells treat the health condition.

In another aspect, some embodiments of the disclosure relate to methods for assisting in the treatment of a health condition (e.g., disease) in an individual in need thereof, the methods including administering to the individual a first therapy including one or more of chimeric polypeptides, synZTE-containing Notch receptors, nucleic acids, recombinant cells, and pharmaceutical compositions as disclosed herein, and a second therapy, wherein the first and second therapies together treat the health condition in the individual. In some embodiments, the methods include administering to the individual a first therapy including an effective number of the recombinant cells as disclosed herein, wherein the recombinant cells treat the health condition.

### Administration of Recombinant Cells to an Individual

In some embodiments, the methods of the disclosure involve administering an effective amount or number of the recombinants cells of the disclosure to an individual in need of such treatment. This administering step can be accomplished using any method of implantation delivery in the art. For example, the recombinant cells can be infused directly in the individual's bloodstream or otherwise administered to the individual.

In some embodiments, the methods disclosed herein include administering, which term is used interchangeably with the terms “introducing,” implanting,” and “transplanting,” recombinant cells into an individual, by a method or route that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even the lifetime of the individual, i.e., long-term engraftment.

When provided prophylactically, the recombinant cells described herein can be administered to an individual in advance of any symptom of a disease or condition to be treated. Accordingly, in some embodiments the prophylactic administration of a recombinant cell population prevents the occurrence of symptoms of the disease or condition.

When provided therapeutically in some embodiments, recombinant cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source. In some embodiments, the recombinant cells are expanded in culture prior to administration to an individual in need thereof.

In some embodiments, the delivery of a recombinant cell composition (e.g., a composition including a plurality of recombinant cells according to any of the cells described herein) into an individual by a method or route results in at least partial localization of the cell composition at a desired site. A composition including recombinant cells can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, delivery by injection or infusion is a standard mode of administration.

In some embodiments, the recombinant cells are administered systemically, e.g., via infusion or injection. For example, a population of recombinant cells are administered other than directly into a target site, tissue, or organ, such that it enters, the individual's circulatory system and, thus, is subject to metabolism and other similar biological processes.

The efficacy of a treatment including any of the compositions provided herein for the treatment of a disease or condition can be determined by a skilled clinician. However, one skilled in the art will appreciate that a treatment is considered effective if any one or all of the signs or symptoms or markers of disease are improved or ameliorated. Efficacy can also be measured by failure of an individual to worsen as assessed by decreased hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

Measurement of the degree of efficacy is based on parameters selected with regard to the disease being treated and the symptoms experienced. In general, a parameter is selected that is known or accepted as correlating with the degree or severity of the disease, such as a parameter accepted or used in the medical community. For example, in the treatment of a solid cancer, suitable parameters can include reduction in the number and/or size of metastases, number of months of progression-free survival, overall survival, stage or grade of the disease, the rate of disease progression, the reduction in diagnostic biomarkers (for example without limitation, a reduction in circulating tumor DNA or RNA, a reduction in circulating cell-free tumor DNA or RNA, and the like), and combinations thereof. It will be understood that the effective dose and the degree of efficacy will generally be determined with relation to a single subject and/or a group or population of subjects. Therapeutic methods of the disclosure reduce symptoms and/or disease severity and/or disease biomarkers by at least about 1, 2, 3, 4, 5, 10, 15, 20, 25, 30, 35, 40, 45, 50, 55, 60, 65, 70, 75, 80, 85, 90, 95, 96, 97, 98, 99, or 100%.

As discussed above, a therapeutically effective amount includes an amount of a therapeutic composition that is sufficient to promote a particular beneficial effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments of the disclosed methods, the individual is a mammal. In some embodiments, the mammal is a human. In some embodiments, the individual has or is suspected of having a disease associated with inhibition of cell signaling mediated by a cell surface ligand or antigen. The diseases suitable for being treated by the compositions and methods of the disclosure include, but are not limited to, cancers, autoimmune diseases, inflammatory diseases, and infectious diseases. In some embodiments, the disease is a cancer or a chronic infection.

### Additional Therapies

As discussed above, the recombinant cells, and pharmaceutical compositions described herein can be administered in combination with one or more additional therapeutic agents such as, for example, chemotherapeutics or anti-cancer agents or anti-cancer therapies. Administration “in combination with” one or more additional therapeutic agents includes simultaneous (concurrent) and consecutive administration in any order. In some embodiments, the one or more additional therapeutic agents, chemotherapeutics, anti-cancer agents, or anti-cancer therapies is selected from the group consisting of chemotherapy, radiotherapy, immunotherapy, hormonal therapy, toxin therapy, and surgery. “Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

### Methods for Modulating an Activity of a Cell

In another aspect, provided herein are various methods for modulating an activity of a cell. The methods include the steps of: (a) providing an effective number of any of the recombinant cells provided herein, and (b) contacting it with a selected ligand, wherein binding of the selected ligand to the extracellular ligand-binding domain results in cleavage of a ligand-inducible proteolytic cleavage site and release of the intracellular domain (ICD) of the chimeric Notch receptor, wherein the release of the ICD results in modulation of an activity of the recombinant cell. One skilled in the art upon reading the present disclosure will appreciate that the disclosed methods can be carried out in vivo, ex vivo, or in vitro. Accordingly, in some embodiments, the contacting of the recombinant cells with the selected ligand is carried out in vivo. In some embodiments, the contacting of the recombinant cells with the selected ligand is carried out ex vivo. In some embodiments, the contacting of the recombinant cells with the selected ligand is carried out in intro. In some embodiments, the release of the ICD results in binding of the ZTE of the released intracellular domain to a ZFA target sequence, which results in modulation of the expression initiation of a polynucleotide of interest, which results in modulation of an activity of the recombinant cell.

Non-limiting exemplary cellular activities that can be modulated using the methods provide herein include, but are not limited to, gene expression, proliferation, apoptosis, non-apoptotic death, differentiation, dedifferentiation, migration, secretion of a gene product, cellular adhesion, and cytolytic activity.

In some embodiments, the released ZTE modulates expression of a gene product of the cell. In some embodiments, the released ZTE modulates expression of a heterologous gene product in the cell. A heterologous gene product is one that is not normally found in the native cell, e.g., not normally produced by the cell. For example, the cell can be genetically modified with a nucleic acid including a nucleotide sequence encoding the heterologous gene product.

In some embodiments, the gene product is a secreted gene product. In some embodiments, the gene product is a cell surface gene product. In some cases, the gene product is an intracellular gene product. In some embodiments, the released transcriptional regulator simultaneously modulates expression of two or more gene products in the cell.

In some embodiments, the gene product in the cell is selected from the group consisting of a chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, an RNA guided nuclease, a site-specific nuclease, a T-cell receptor (TCR), a chimeric antigen receptor (CAR), a toxin, a toxin-derived protein, a transcriptional regulator, a transcriptional activator, a transcriptional repressor, a translation regulator, a translational activator, a translational repressor, an activating immuno-receptor, an antibody, an apoptosis inhibitor, an apoptosis inducer, an engineered T cell receptor, an immuno-activator, an immuno-inhibitor, an immune cell receptor, and an inhibiting immuno-receptor. In some embodiments, the immune cell receptor is a T-cell receptor (TCR). In some embodiments, the immune cell receptor is a chimeric antigen receptor (CAR).

In some embodiments, the released transcriptional regulator modulates differentiation of the cell. In some embodiments, the cell is an immune cell, a stem cell, a progenitor cell, or a precursor cell.

The chimeric receptors and synZTE-containing Notch receptors of the present disclosure provide a higher degree of expression than an existing first-generation SynNotch receptor, when using identical binding domains and ICDs. Depending on the ligand/binding domain pair and their affinity, the chimeric polypeptides and synZTE-containing Notch receptors of the disclosure can provide expression enhancement of about 10%, about 15%, about 20%, about 25%, about 30%, about 35%, about 40%, about 45%, or about 50% higher than a corresponding first-generation SynNotch receptor.

Additionally, the chimeric receptors and synZTE-containing Notch receptors of the disclosure can provide transcriptional regulation that responds to the degree of T cell activation, independent of ligand binding. For example, when expressed in a T cell, some receptors of the disclosure provide a stronger ligand-induced signal when the T-cell is activated as compared to the ligand-induced signal when the T-cell is not activated. This permits additional flexibility in use, for example in cases where it is desired to enhance or suppress a T cell response when activated despite the absence of the chimeric receptor ligand.

### Kits

Also provided herein are various kits for the practice of a method described herein. A kit can include one or more of the chimeric polypeptides, synZTE-containing Notch receptors, recombinant nucleic acids, recombinant cells, or pharmaceutical compositions as provided and described herein. For example, provided herein, in some embodiments, are kits that include one or more of: a chimeric polypeptide as described herein, a synZTE-containing Notch receptor as described herein, a recombinant nucleic acids as described herein, a recombinant cell as described herein, or a pharmaceutical composition as described herein.

In other examples, provided herein are kits that include: (a) a chimeric polypeptide of the disclosure; (b) a recombinant nucleic acid of the disclosure; and (c) an engineered response element including: (i) a ZFA target sequence; (ii) an engineered responsive promoter operably linked to the ZFA target sequence; and (iii) a polynucleotide of interest; wherein binding of the ZTE to the nucleic acid target sequence modulates transcription initiation of the polynucleotide of interest.

In some embodiments, the kits of the disclosure further include one or more syringes (including pre-filled syringes) and/or catheters (including pre-filled syringes) used to administer one any of the recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the chimeric receptor polypeptides.

In some embodiments, the components of a kit can be in separate containers. In some other embodiments, the components of a kit can be combined in a single container.

In some embodiments, a kit can further include instructions for using the components of the kit to practice the methods disclosed herein. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate. No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature cited above.

Additional embodiments are disclosed in further detail in the following examples, which are provided by way of illustration and are not in any way intended to limit the scope of this disclosure or the claims.

### Example 1

### Design and Construction of Chimeric Receptor and Response Element Constructs

This Example describes the design and construction of a family of chimeric Notch receptors with zinc finger-containing transcriptional effector. Detailed information for various exemplary receptors of the disclosure can be found in Tables 1 and 2 below.

TABLE 1
This table provides a brief description for each of the chimeric Notch receptors,
their corresponding components, as well as corresponding sequence identifiers
as set forth in the Sequence Listing. ECD: extracellular domain; N-JMD: N-terminal
juxtamembrane domain; TMD: transmembrane domain; STS: stop-transfer-sequence;
TF: zinc finger-containing transcriptional effector (synZTE).

| | Receptor | | | | | | Full |
|---|---|---|---|---|---|---|---|
| Construct ID | Description | ECD | N-JMD | TMD | STS | TF | sequence |
| pRay050Z3 | pRay050 with ZF3 | SEQ ID | SEQ ID | SEQ ID | SEQ ID | SEQ ID | SEQ ID |
| | replacing G4VP64 | NO: 10 | NO: 11 | NO: 31 | NO: 39 | NO: 56 | NO: 1 |
| pIZ621Z3 | “miniNotch1” with | SEQ ID | SEQ ID | SEQ ID | SEQ ID | SEQ ID | SEQ ID |
| | Notch2 STS and ZF3 | NO: 10 | NO: 11 | NO: 31 | NO: 40 | NO: 56 | NO: 2 |
| | replacing G4VP64 | | | | | | |
| pIZ343Z3 | truncated CD8 Hinge | SEQ ID | SEQ ID | SEQ ID | SEQ ID | SEQ ID | SEQ ID |
| | receptor with Notch1 | NO: 10 | NO: 21 | NO: 31 | NO: 39 | NO: 56 | NO: 3 |
| | TMD and STS, with | | | | | | |
| | ZF3 replacing | | | | | | |
| | G4VP64 | | | | | | |
| pIZ361Z3 | truncated CD8 Hinge | SEQ ID | SEQ ID | SEQ ID | SEQ ID | SEQ ID | SEQ ID |
| | receptor with Notch1 | NO: 10 | NO: 21 | NO: 31 | NO: 40 | NO: 56 | NO: 4 |
| | TMD and Notch2 | | | | | | |
| | STS, with ZF3 | | | | | | |
| | replacing G4VP64 | | | | | | |
| pDP1158 | truncated CD8 Hinge | SEQ ID | SEQ ID | SEQ ID | SEQ ID | SEQ ID | SEQ ID |
| | receptor with Notch1 | NO: 10 | NO: 21 | NO: 31 | NO: 40 | NO: 55 | NO: 5 |
| | TMD and Notch2 | | | | | | |
| | STS, with ZF2 | | | | | | |
| | replacing G4VP64 | | | | | | |
| pDP1159 | truncated CD8 Hinge | SEQ ID | SEQ ID | SEQ ID | SEQ ID | SEQ ID | SEQ ID |
| | receptor with Notch1 | NO: 10 | NO: 21 | NO: 31 | NO: 40 | NO: 57 | NO: 6 |
| | TMD and Notch2 | | | | | | |
| | STS, with ZF4 | | | | | | |
| | replacing G4VP64 | | | | | | |
| pDP1160 | truncated CD8 Hinge | SEQ ID | SEQ ID | SEQ ID | SEQ ID | SEQ ID | SEQ ID |
| | receptor with Notch1 | NO: 10 | NO: 21 | NO: 31 | NO: 40 | NO: 58 | NO: 7 |
| | TMD and Notch2 | | | | | | |
| | STS, with ZF6 | | | | | | |
| | replacing G4VP64 | | | | | | |
| pDP1161 | truncated CD8 Hinge | SEQ ID | SEQ ID | SEQ ID | SEQ ID | SEQ ID | SEQ ID |
| | receptor with Notch1 | NO: 10 | NO: 21 | NO: 31 | NO: 40 | NO: 59 | NO: 8 |
| | TMD and Notch2 | | | | | | |
| | STS, with ZF10 | | | | | | |
| | replacing G4VP64 | | | | | | |
| pDP1162 | truncated CD8 Hinge | SEQ ID | SEQ ID | SEQ ID | SEQ ID | SEQ ID | SEQ ID |
| | receptor with Notch1 | NO: 10 | NO: 21 | NO: 31 | NO: 40 | NO: 60 | NO: 9 |
| | TMD and Notch2 | | | | | | |
| | STS, with Z11 | | | | | | |
| | replacing G4VP64 | | | | | | |

TABLE 2
This table provides a brief description for each of the chimeric Notch receptors and
the respective components (with components separated by hyphens). Unless otherwise
noted, the entry refers to a protein of human origin. For example, “Notch1, Notch1”
indicates that two sequence from Notch1 were fused to generate this protein module.

| Construct | | | | | | |
|---|---|---|---|---|---|---|
| ID | Receptor Description | ECD | N-JMD | TMD | STS | TF |
| pRay050Z3 | antiCD19scFv- | CD8a signal | Notch1, | Notch1 | Notch1 | synthetic |
| | Notch1deltaNRR-ZF3 | peptide, myc-tag, | Notch1 | | | zinc finger |
| | | anti-CD19 scFv | | | | ZF3 |
| pIZ621Z3 | antiCD19scFv- | CD8a signal | Notch1, | Notch1 | Notch2 | synthetic |
| | Notch1deltaNRR- | peptide, myc-tag, | Notch1 | | | zinc finger |
| | Notch2STS-ZF3 | anti-CD19 scFv | | | | ZF3 |
| pIZ343Z3 | antiCD19scFv-CD8Hinge2- | CD8a signal | truncated | Notch1 | Notch1 | synthetic |
| | Notch1TMD-ZF3 | peptide, myc-tag, | CD8 hinge | | | zinc finger |
| | | anti-CD19 scFv | | | | ZF3 |
| pIZ361Z3 | antiCD19scFv-CD8Hinge2- | CD8a signal | truncated | Notch1 | Notch2 | synthetic |
| | Notch1TMD-Notch2STS- | peptide, myc-tag, | CD8 hinge | | | zinc finger |
| | ZF3 | anti-CD19 scFv | | | | ZF3 |
| pDP1158 | antiCD19scFv-HingeNotch | CD8a signal | truncated | Notch1 | Notch2 | synthetic |
| | ZF2 p65 (Notch1TMD- | peptide, myc-tag, | CD8 hinge | | | zinc finger |
| | Notch2STS) | anti-CD19 scFv | | | | ZF2 |
| pDP1159 | antiCD19scFv-HingeNotch | CD8a signal | truncated | Notch1 | Notch2 | synthetic |
| | ZF4 p65 (Notch1TMD- | peptide, myc-tag, | CD8 hinge | | | zinc finger |
| | Notch2STS) | anti-CD19 scFv | | | | ZF4 |
| pDP1160 | antiCD19scFv-HingeNotch | CD8a signal | truncated | Notch1 | Notch2 | synthetic |
| | ZF6 p65 (Notch1TMD- | peptide, myc-tag, | CD8 hinge | | | zinc finger |
| | Notch2STS) | anti-CD19 scFv | | | | ZF6 |
| pDP1161 | antiCD19scFv-HingeNotch | CD8a signal | truncated | Notch1 | Notch2 | synthetic |
| | ZF10p65 (Notch1TMD- | peptide, myc-tag, | CD8 hinge | | | zinc finger |
| | Notch2STS) | anti-CD19 scFv | | | | ZF10 |
| pDP1162 | antiCD19scFv-HingeNotch | CD8a signal | truncated | Notch1 | Notch2 | synthetic |
| | ZF11 p65 (Notch1TMD- | peptide, myc-tag, | CD8 hinge | | | zinc finger |
| | Notch2STS) | anti-CD19 scFv | | | | ZF11 |

The chimeric receptors described in Tables 1-2 above were built by fusing a single-chain antigen-binding fragment CD19 scFv (Porter D L et al., 2011) to the corresponding receptor scaffold and a synZF protein domain fused to the human p65 transactivator.

The synthetic zinc finger-containing transcriptional effectors (synZTE) used in these experiments were minimal, modular fusions of DNA binding and effector domains that together could locally regulate the expression of genes at responsive promoters containing specific target binding sequences. The amino acid sequences of six exemplary synZTE (ZF2, ZF3, ZF4, ZF6, ZF10, and ZF11) are provided as SEQ ID NOS: 55-60 in the Sequence Listing. The engineered zinc finger (ZF) DNA binding arrays were coupled to a transcriptional effector domain (a p65 activation domain of NFκB). The engineered ZF arrays described herein were derived from native mammalian ZF scaffolds, but re-designed to target specific 18-20 nucleotide sequences that were demonstrated to be unique and orthogonal from human genome sequences. Thus, it was expected that this feature could confer reduced off-target binding and regulation potential in the human genome. The nucleotide sequences of the ZTE target binding sequences are also provided as SEQ ID NOS: 61-71 in the Sequence Listing. Corresponding responsive promoters were then designed by placing tandem ZF binding sites upstream of constitutive promoters to enable regulated gene expression control in mammalian cells. The transcriptional effector domain used in these experiments was a p65 transcriptional activator domain from NFκB.

All receptors contained an N-terminal CD8α signal peptide (SEQ ID NO: 72) for membrane targeting and a myc-tag (SEQ ID NO: 73) for suitable determination of surface expression with an antibody conjugated to a fluorescent dye (α-myc A647®, Cell Signaling Technology, Cat #2233). For the construction of these receptors, DNA fragments coding for the amino acid sequences provided in Table 1 and Sequence Listing were PCR amplified from synthesized gene fragments or plasmids containing DNA sequence for the indicated protein, and assembled using standard cloning techniques (e.g., overhang PCR, fusion PCR, and In-fusion cloning) with flanking translation start and stop sequences, into the modified lentiviral expression vector pHR′ SIN:CSW vector (K T Roybal et al., Cell (2016 Oct. 6) 167(2):419-32), which contained a phosphoglycerate kinase (PGK) promoter for all primary T cell experiments described in Example 6 below.

The pHR′ SIN:CSW vector was also modified to produce the response element plasmids. For this purpose, eight copies of the specific zinc finger target sequences were cloned 5′ to a minimal ybTATA promoter. The resulting target DNA stretches named ZF2, ZF3, ZF4, ZF6, ZF10, and ZF11. The nucleotide sequences and amino acid sequence encoded thereby are also provided in the Sequence Listing (SEQ ID NOS: 61-71). Also included in the response element plasmids was a PGK promoter that constitutively drives expression of a yellow fluorescent reporter protein (mCitrine) to suitably identify successfully transduced T cells.

For the construction of all inducible BFP vectors, the original ZF3-containing vector was modified between EcoRI and BamHI sites with a PCR-amplified copy of the corresponding zinc finger target site. All constructs were assembled using NEB HiFi Assembly (E2621S) according to the manufacturer's instructions.

### Example 2

### Primary Human T-Cell Isolation and Culture

This Example describes the isolation and culture of primary human T cells that were subsequently used in various cell transduction experiments described in Example 3 below.

In these experiments, primary CD4⁺ and CD8⁺ T cells were isolated from blood after apheresis and enriched by negative selection using human T-cell isolation kits (human CD4⁺ or CD8⁺ enrichment cocktail; STEMCELL Technologies Cat #15062 and 15063). Blood was obtained from Blood Centers of the Pacific (San Francisco, Calif.) as approved by the University Institutional Review Board. T cells were cryopreserved in growth medium (RPMI-1640, UCSF cell culture core) with 20% human AB serum (Valley Biomedical Inc., #HP1022) and 10% DMSO. After thawing, T cells were cultured in human T cell medium containing X-VIVO™ 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

### Example 3

### Human T Cells were Stably Transduced with Lentiviral Vectors

The Example describes a general protocol used for lentiviral transduction of human T cells.

Generally, lentiviral vectors pseudo-typed with vesicular stomatitis virus envelope G protein (VSV-G) (pantropic vectors) were produced via transfection of Lenti-X™ 293T cells (Clontech #11131D) with a pHR′ SIN:CSW transgene expression vector and the viral packaging plasmids pCMVdR8.91 and pMD2.G using Mirus TransIT®-Lenti (Mirus, #MIR 6606). Generally, primary T cells were thawed the same day and, after 24 hours in culture, were stimulated with beads having anti-CD3 and anti-CD28 antibodies bound to the surface (Human T-Activator CD3/CD28 Dynabeads®, Life Technologies #11131D) at a 1:3 cell:bead ratio. At 48 hours, viral supernatant was harvested and the primary T cells were exposed to the virus for 24 hours. At Day 5 post T-cell stimulation, the Dynabeads were removed, and the T cells expanded until Day 10 when they were rested and could be used in assays. T cells were sorted for assays with a Becton Dickinson (BD) FACSAria™ II flow cytometer.

### Example 4

### Cancer Cell Lines

The cancer cell lines used were K562 myelogenous leukemia cells (ATCC #CCL-243). K562 cells were lentivirally transduced to stably express human CD19 at equivalent levels as Daudi tumors. CD19 levels were determined by staining the cells with anti-CD19 APC (Biolegend #302212). All cell lines were sorted for expression of the transgenes.

### Example 5

### Generation of Reporter Jurkat T Cells

This Example describes the generation of reporter Jurkat T cells that were subsequent used for the testing of various ZTE-containing Notch receptors described herein.

In these experiments, E6-1 Jurkat T cells (ATCC #TIB-152) were lentivirally transduced with a reporter plasmid carrying an inducible BFP reporter gene and a constitutive mCitrine reporter gene, as described previously (K. T. Roybal et al., Cell, 164:1-10, 2016). Reporter-positive Jurkat cells were sorted for mCitrine expression using a Beckton Dickinson (BD) FACSAria™ II flow cytometer and expanded.

Lentiviral particles were produced with the receptor transgene expression vector as described previously (L. Morsut et al., Cell (2016) 164:780-91). Reporter-positive Jurkat cells were transduced with individual receptors and expanded for experimentation in 96 well plates.

### Example 6

### In Vitro Stimulation of Primary T Cells

This Example describes a general protocol used to demonstrate the stimulation of primary T cells in vitro by the chimeric synZTE-containing Notch receptors described herein.

For all in vitro T-cell stimulations, 1×105 T cells were co-cultured with sender cells at a 1:1 ratio in flat bottom 96-well tissue culture plates. The cultures were analyzed at 24 hours for reporter activation with a BD Fortessa™ X-50. All flow cytometry analysis was performed in FlowJo™ software (TreeStar, Inc.).

### Example 7

### Preliminary Characterization of synZTE-Containing Human Notch Receptors

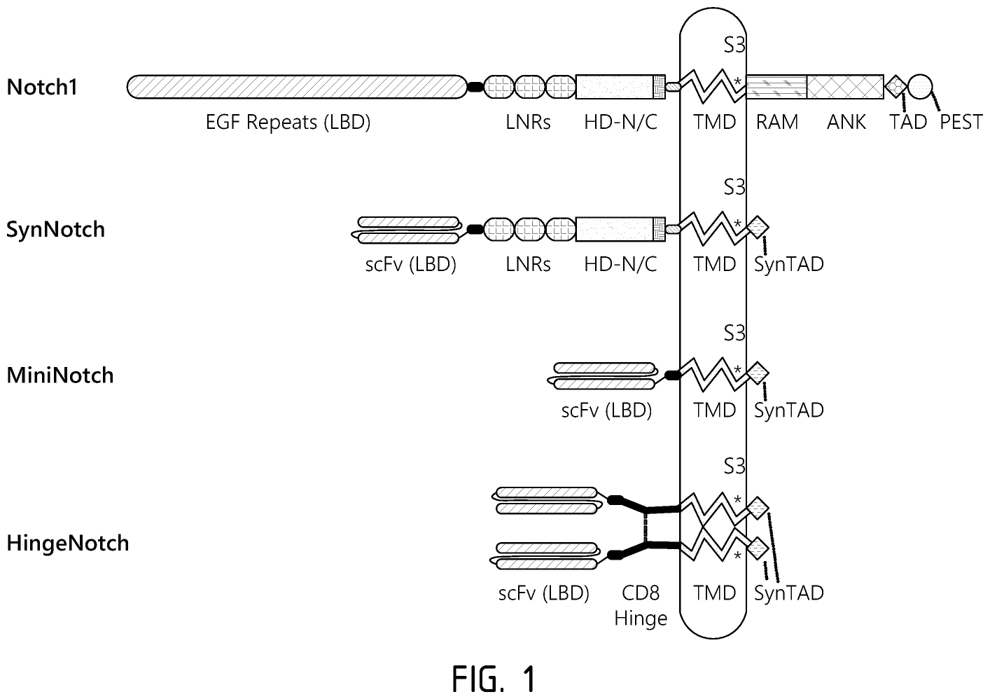

This Example describes the results of experiments performed to evaluate functionality of engineered synZTE-containing human Notch receptors. As schematically illustrated in FIG. 1, existing first-generation SynNotch variants are intended to rely on the core force-sensing module of Notch (the Notch regulatory region, NRR, stretching of which leads to ligand-inducible cleavage at the S3 site) to regulate customizable intracellular transactivators with user-defined ligand binding domains. In MiniNotch variants, much of the NRR is further truncated. HingeNotch variants additionally feature disulfide-mediated oligomerization due to the insertion of a hinge domain (e.g., a hinge domain from CD8).

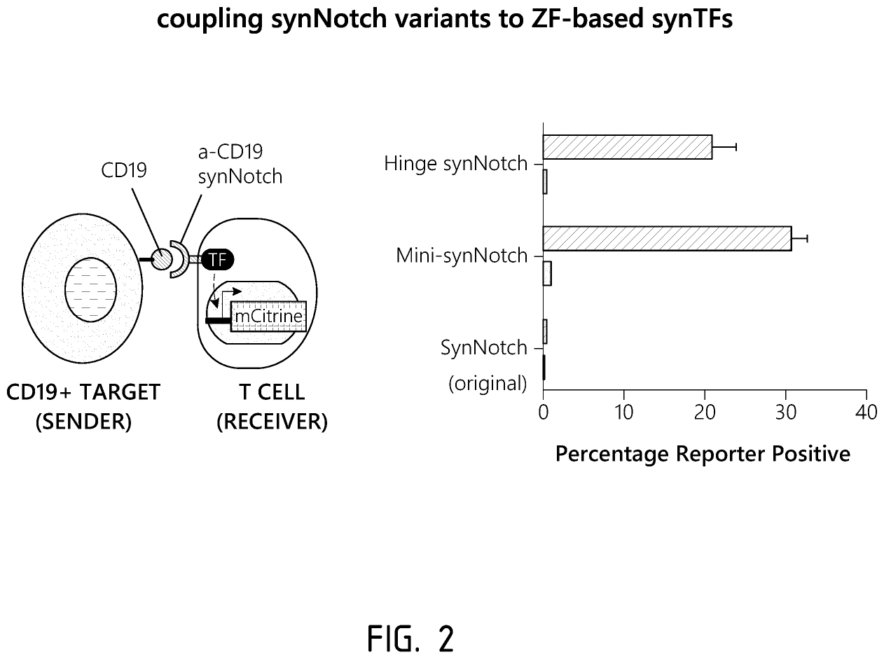

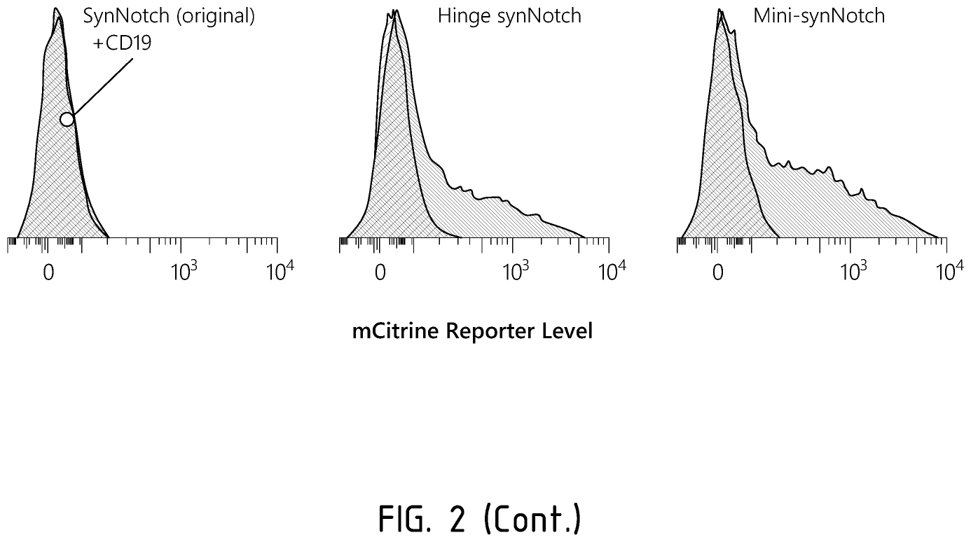

Three variants of synZTE-containing human Notch receptors were constructed by coupling each of the three engineered Notch receptor variants described in FIG. 1 with a synthetic zinc finger-based transcriptional effector (synZTE). As shown in FIG. 2, all of the newly constructed synZTE-containing Notch receptors contained an anti-CD19 scFv and were placed under control of a phosphoglycerate kinase (PGK) promoter. In the experiments, an 8×ZF3 response element with a synthetic ybTATA promoter was used to produce mCitrine upon activation. These experiments were all performed in a reporter+ Jurkat line that was not previously sorted. The percentage of reporter positive cells presented in FIG. 2 was calculated by dividing the +citrine positive cells by the +receptor (myc) positive cells. Separately, various response element numbers were also tested, where 8×ZF3 performs better than 4×ZF3, but 12×ZF3 and 16×ZF3 did not further improve response.

### Example 8

### Assessing Functionality of Engineered ZTE-Containing Human SynNotch Receptors

This Example describes the results of experiments performed to evaluate functionality of the new synZTE-containing human SynNotch receptors. Two variants of ZTE-containing human SynNotch receptors were constructed.

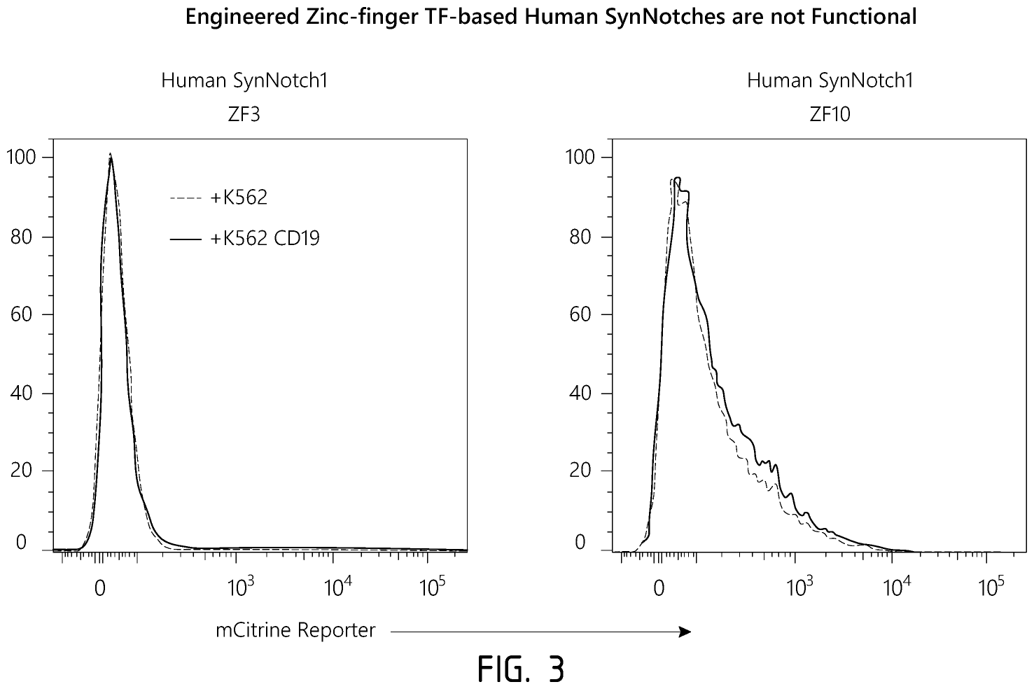

As shown in FIG. 3, an exemplary engineered human SynNotch receptor containing the synthetic zinc finger-containing transcriptional activator Z3 is shown on the left panel, which was designed based upon human Notch1 proteins. The right panel shows another exemplary engineered human SynNotch receptor containing the synthetic zinc finger-containing transcriptional activator Z10. In these experiments, Jurkat T-cells were transduced with anti-CD19 synZTE-containing Notch receptors containing either ZF3 or ZF10 with unique DNA binding specificities, along with their cognate mCitrine reporter. Reporter expression levels indicating receptor activation with antigen-negative vs. antigen-positive K562 cells was assessed after 24 hours of co-incubation. As shown in FIG. 3, both anti-CD19 synZTE-containing Notch receptors containing either ZF3 or ZF10 failed to activate expression of the reporter gene mCitrine.

The results described in this Example demonstrate that engineered ZTE-containing human SynNotch receptors fail to activate transcription of the reporter gene, unlike the Hinge-Notch receptors of the disclosure.

### Example 9

### ZTE-Containing Hinge-Notch Receptor Activation in Primary CD4⁺ T-Cells

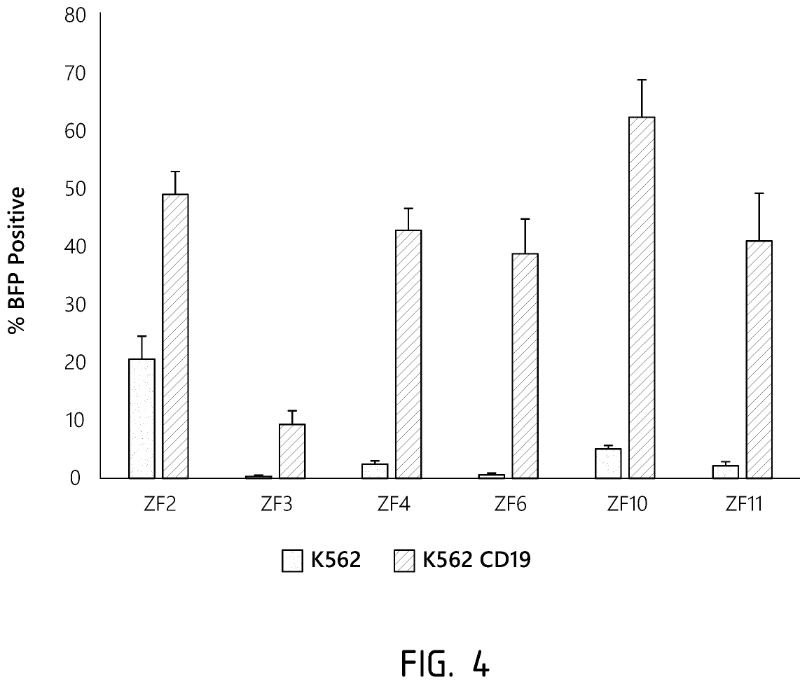

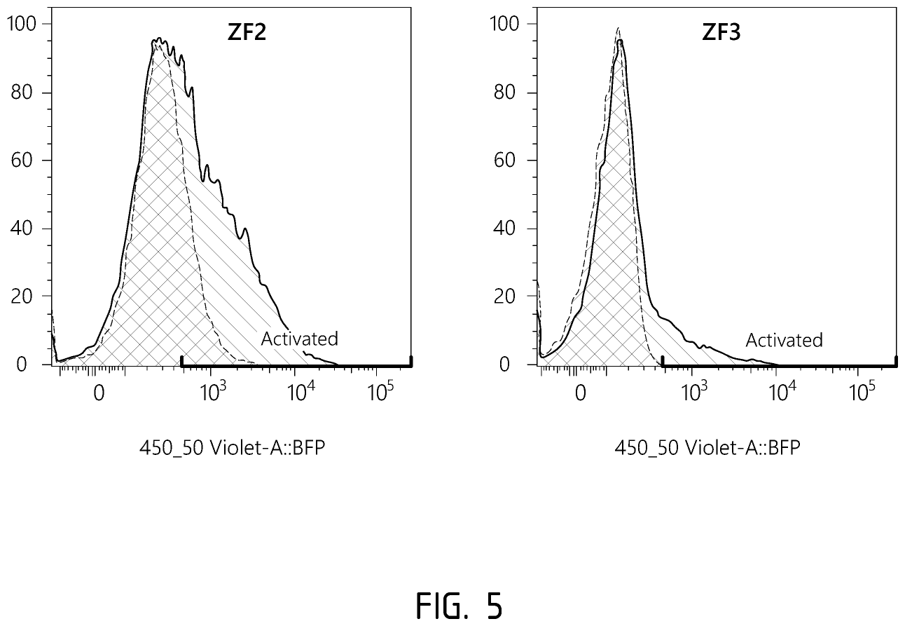

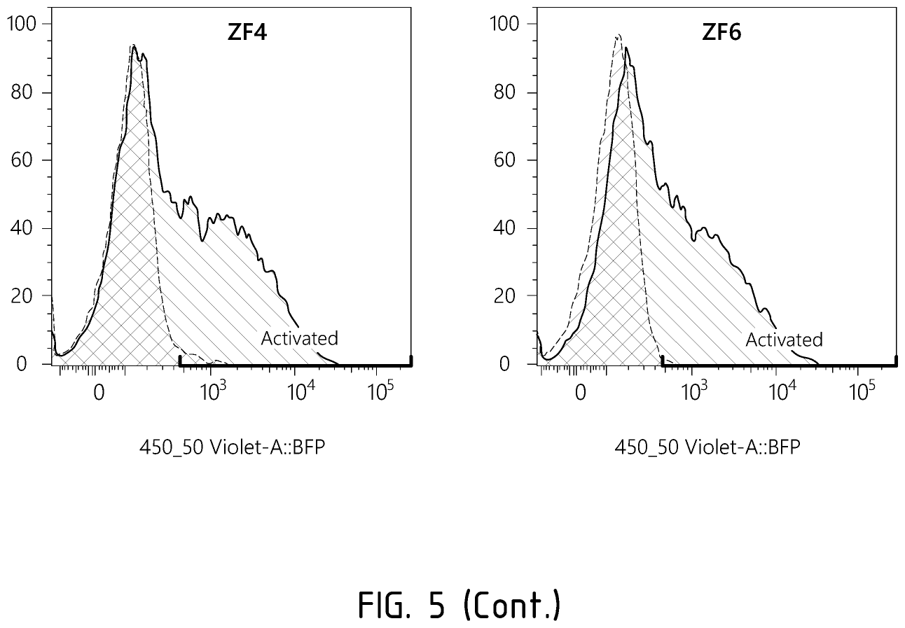

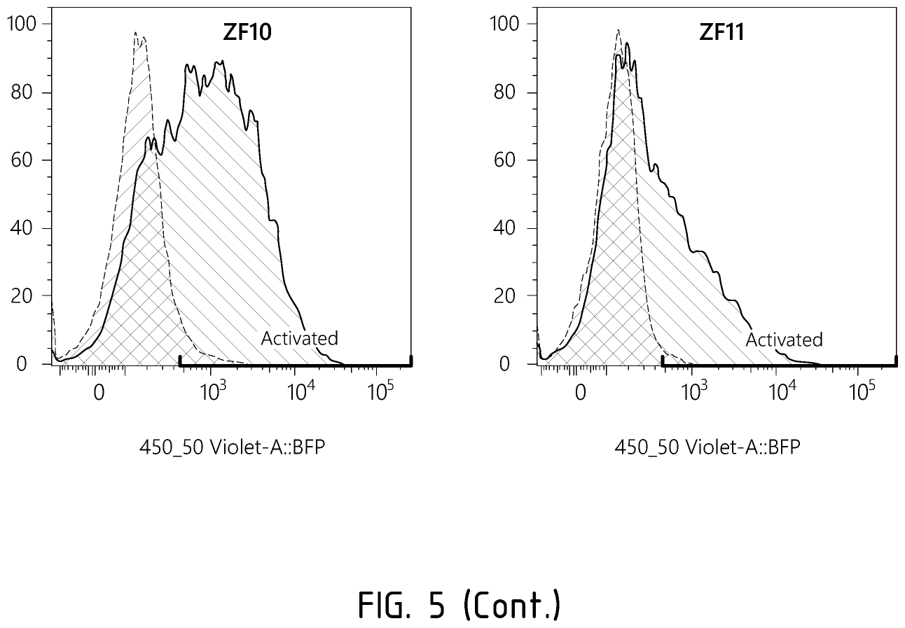

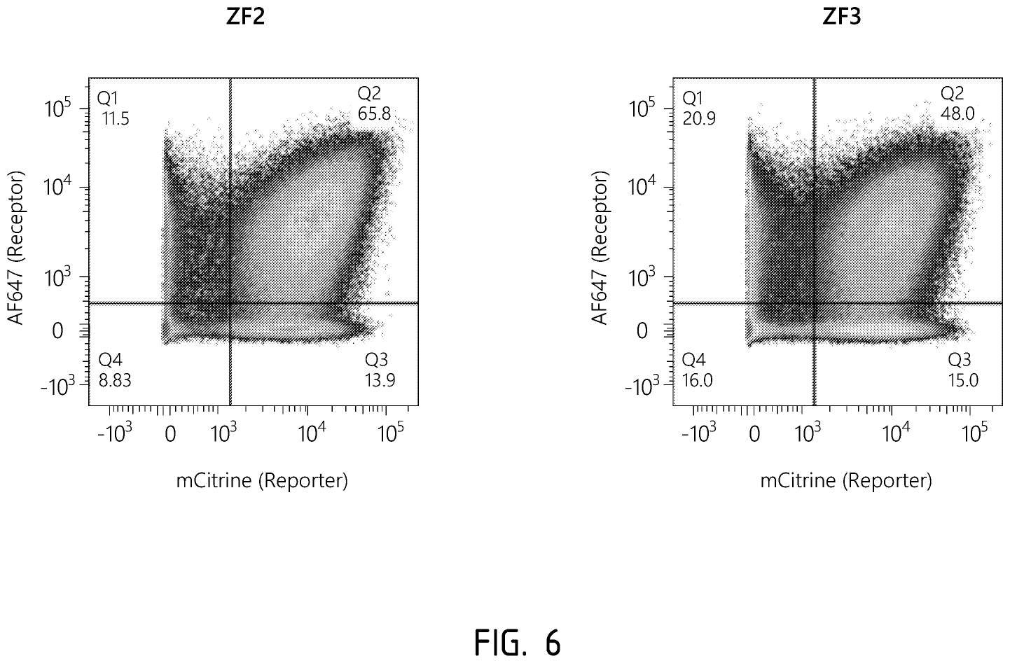

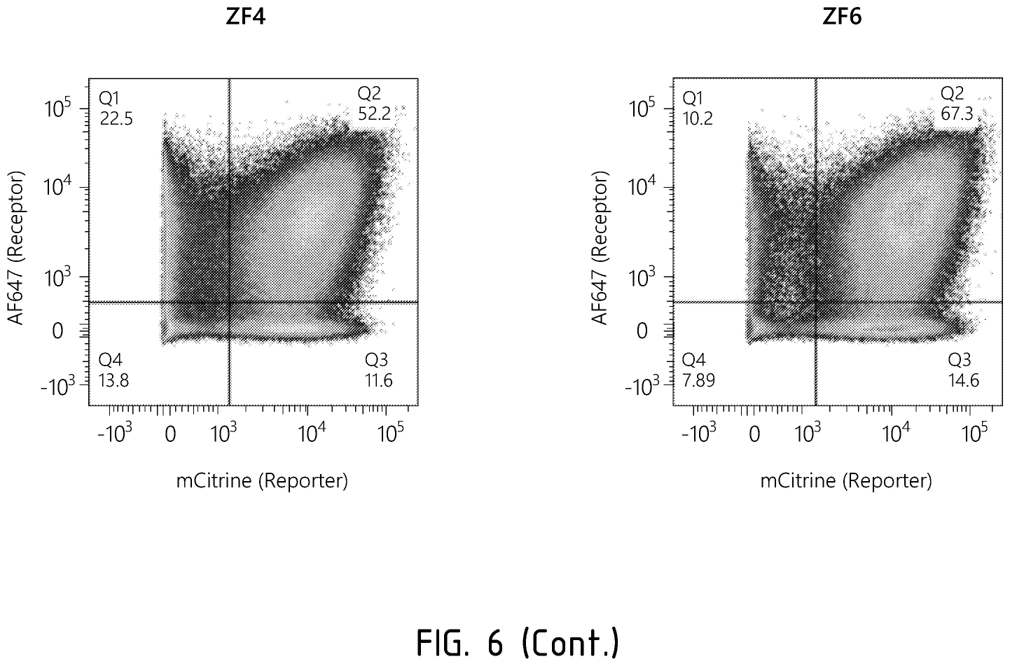

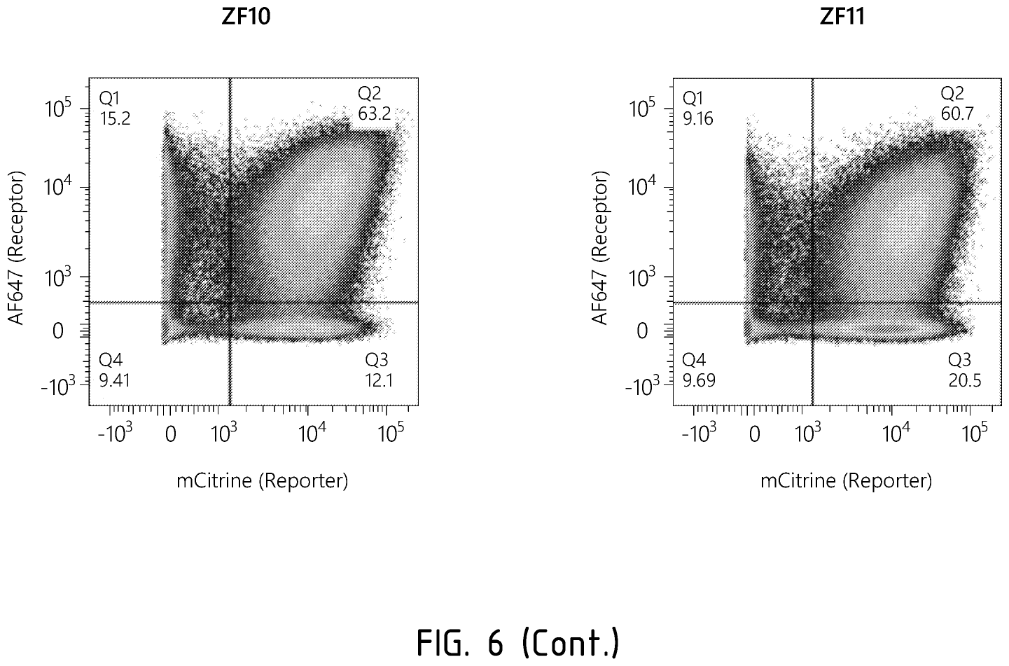

This Example describes the results of experiments performed to demonstrate gene activation mediated by novel synZTE-containing Hinge-Notch receptors described herein in primary CD4⁺ cells. These experiments were conducted using six exemplary ZTE-containing Hinge Notch receptors which contained an anti-CD19scFv and one of the following synthetic zinc finger-containing transcriptional activators (synTFs): ZF2, ZF3, ZF4, ZF6, ZF10, and ZF11. In these experiments, primary CD4+ T-cells from two different donors were transduced with anti-CD19 ZTE-containing Hinge Notch receptors containing one of six different SynTF transactivators with unique DNA binding specificities, along with their cognate BFP-expressing reporters. Reporter expression levels indicating receptor activation with antigen-negative vs. antigen-positive K562 cells was assessed after 24 hours of co-incubation (N=2; error bars represent standard deviation). As shown in FIG. 4, the expression of the reporter gene BFP was activated by all six ZTE-containing Hinge Notch receptors. FIG. 5 shows normalized fluorescence activation profiles of the T-cells described in FIG. 4 co-incubated with antigen-negative (red) or antigen-positive (blue) K562 target cells. In addition, expression levels of the receptor (vertical axis) vs. the cognate reporter (horizontal axis) for these six ZTE-containing Hinge Notch receptors were also illustrated in FIG. 6.

The results described in this Example demonstrate that engineered ZTE-containing Hinge-Notch receptors functionally activate transcription of the reporter gene in response to antigen stimulation of the receptor.

### Example 10

### Optimization of synZTE-Containing HingeNotch Receptors

This Example describes the results of experiments performed to further optimize synZTE-containing HingeNotch receptors. In these experiments, a number of synZTE-containing HingeNotch variants were constructed by removing various additional peptide sequences therefrom and subsequently tested in Jurkat T-cells.

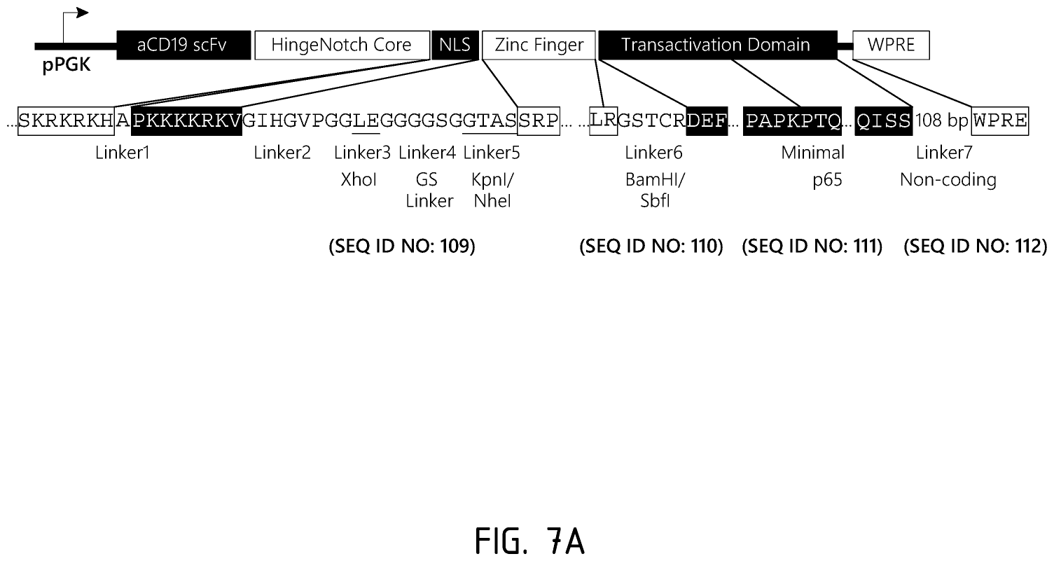

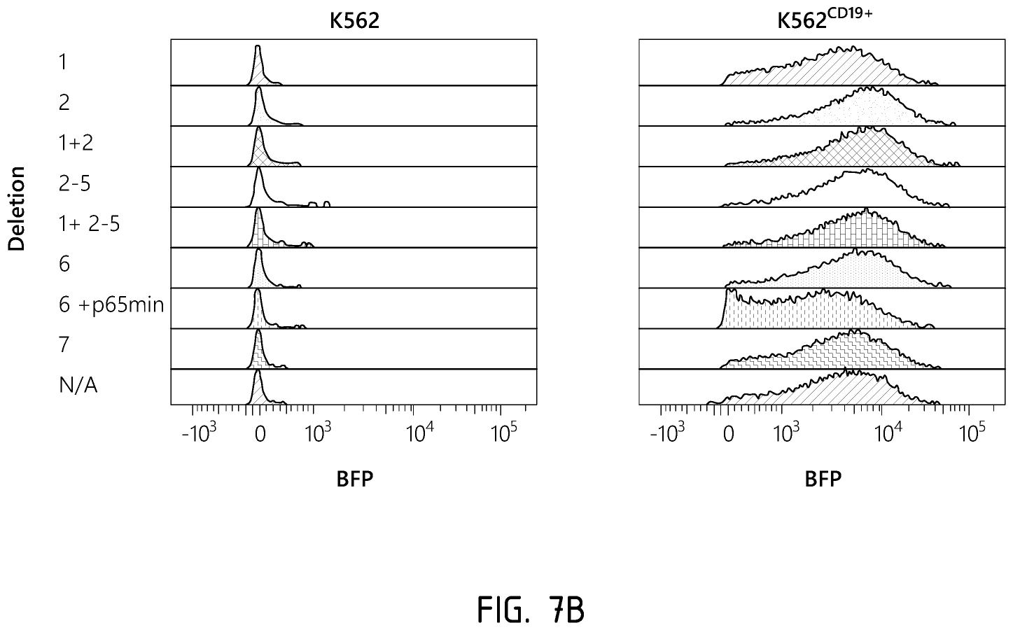

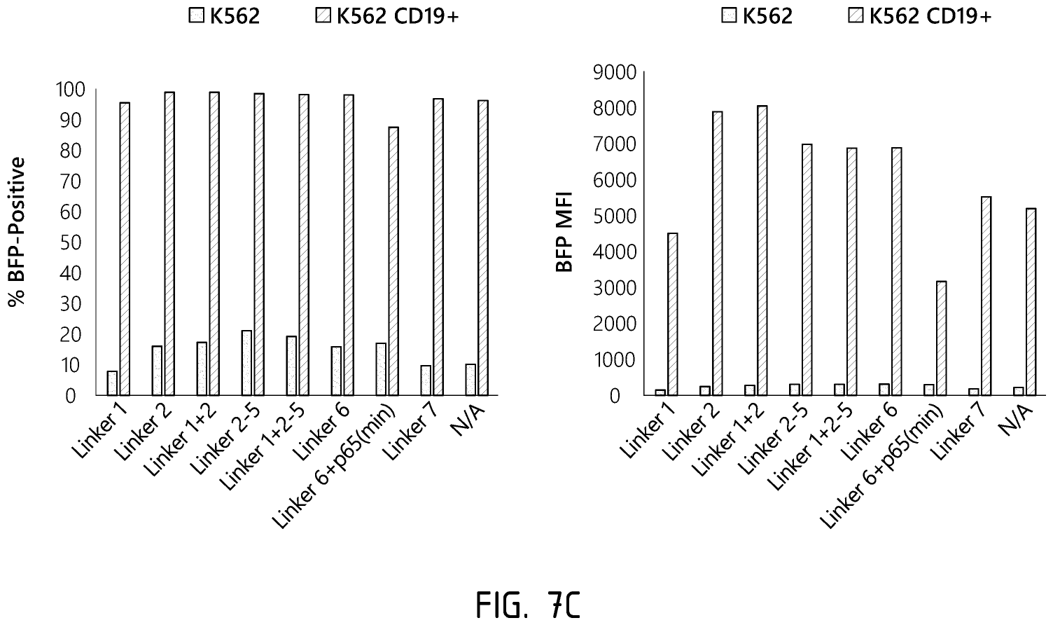

As shown in FIG. 7A is a sequence schematic of loci within a lentiviral expression construct for an exemplary synZTE-containing HingeNotch ZF6, i.e., pDP1160 (SEQ ID NO: 7), that were interspersed with functionally unannotated sequences. These include (i) an alanine between the HingeNotch core functional region and the nuclear localization sequence (NLS) of the synZTE-containing HingeNotch (Linker 1), (ii) several potentially non-essential regions between the NLS and zinc-finger domain consisting of a polypeptide (Linker 2), (iii) the expression product of an XhoI restriction enzyme site (Linker 3), (iv) a flexible linker glycine-serine (Linker 4), (v) the expression product KpnI and NheI restriction enzyme sites (Linker 5), and also (vi) the expression product of BamHI and SbfI site restriction enzyme sites between the zinc finger and transactivation domain p65 (Linker 6)). Additionally, the 108 bp between the p65 transactivation domain and the WPRE were replaced with an 8 bp NotI site (Linker 7). Moreover, in one construct, the full-length transactivation domain p65 was replaced with a minimal sequence starting at residue P68. The description and amino acid sequences of these anti-CD19 HingeNotch-ZF6 receptors bearing the indicated linker deletions or modifications are provided in Table 3 below and the Sequence Listing.

TABLE 3
This table provides a brief description for exemplary synZTE-containing
HingeNotch variants with their respective components.

| Construct | | | | SEQ ID |
|---|---|---|---|---|
| ID | Receptor Description | FIG. | Figure sub-region | NO |
| pDP1160 | anti-CD19 HingeNotch ZF6 p65 | 7 and 8 | 7B and 7C “N/A” | 7 |
| | | | (“Original” Construct), | |
| | | | 8 Panels A-C (“ZF6 | |
| | | | Original”) | |
| pDP1233 | anti-CD19 HingeNotch ZF6 p65 ΔA | 7 | 7B and 7C “Deletion 1” | 113 |
| pDP1234 | anti-CD19 HingeNotch ZF6 p65 | 7 | 7B and 7C “Deletion 2” | 114 |
| | Δ3xFlagseq | | | |
| pDP1235 | anti-CD19 HingeNotch ZF6 p65 | 7 | 7B and 7C “Deletion | 115 |
| | Δ3xFlagseq + A | | 1 + 2” | |
| pDP1236 | anti-CD19 HingeNotch ZF6 p65 | 7 | 7B and 7C “Deletion | 116 |
| | Δ3xFlagseq + GS Linker | | 2-5” | |
| pDP1237 | anti-CD19 HingeNotch ZF6 p65 | 7 | 7B and 7C “Deletion | 117 |
| | Δ3xFlagseq + GS Linker + A | | 1 + 2-5” | |
| pDP1238 | anti-CD19 HingeNotch ZF6 p65 | 7 | 7B and 7C “Deletion 6” | 118 |
| | ΔBamHI-TCR Linker | | | |
| pDP1239 | anti-CD19 HingeNotch ZF6 min-p65 | 7 | 7B and 7C “Deletion | 119 |
| | ΔBamHI | | 6 + p65 min” | |
| pDP1261 | anti-CD19 HingeNotch ZF6 p65 | 7 | 7B and 7C “Deletion 7” | 120 |
| | ΔPre-WPRE | | | |
| PDP1161 | anti-CD19 HingeNotch ZF10 p65 | 8 | 8A-8C (″ZF10 original) | 8 |
| pDP1306 | anti-CD19 HingeNotch ZF6 p65 | 8 | 8A-8C (″ZF6 | 121 |
| | (Minimized) ΔPre-WPRE | | Minimized) | |
| pDP1307 | anti-CD19 HingeNotch ZF10 p65 | 8 | 8A-8C (″ZF10 | 122 |
| | (Minimized) ΔPre-WPRE | | minimized) | |
| pIZ621Z3 | anti-CD19 MiniNotch ZF3 p65 | 9 | 9A and 9B “SV40 | 2 |
| | | | NLS” | |
| pDP1008 | anti-CD19miniNotch ZF3 single Notch1 | 9 | 9A and 9B “hNotch1 | 123 |
| | NLS | | NLS” | |

FIG. 7B summarizes BFP expression from Jurkat cells transduced with a ZF6BD-BFP reporter construct and a panel of anti-CD19 HingeNotch-ZF6 expression vectors bearing the indicated linker deletions or modifications. In these experiments, cells were stimulated with unmodified K562 cells (left panel) or CD19-expression K562 cells (right panel). FIG. 7C depicts percent BFP-expressing Jurkat cells (left panel) and BFP MFI (right panel) tabulated for the data presented in FIG. 7B. It was observed that all truncated receptors, with the exception of ΔLinker 1, demonstrated augmented activation relative to the original full-length receptor, i.e., pDP1160. The truncation of the p65 transactivation domain to p65 min demonstrated a noticeable decrease in activation, resulting in subsequent “fully minimized” construct designs that retain the full length transactivation domain p65.

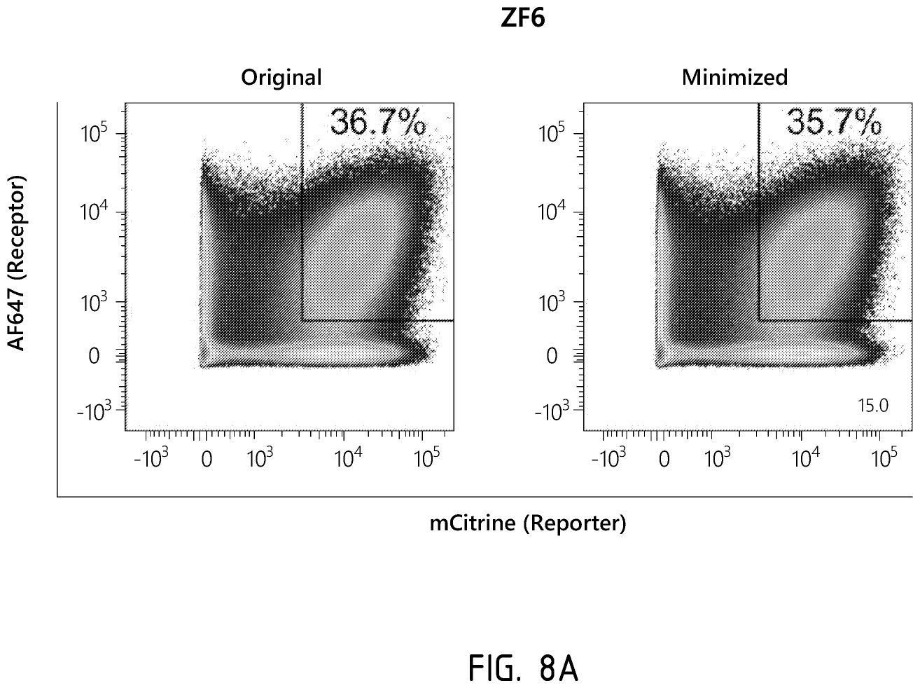

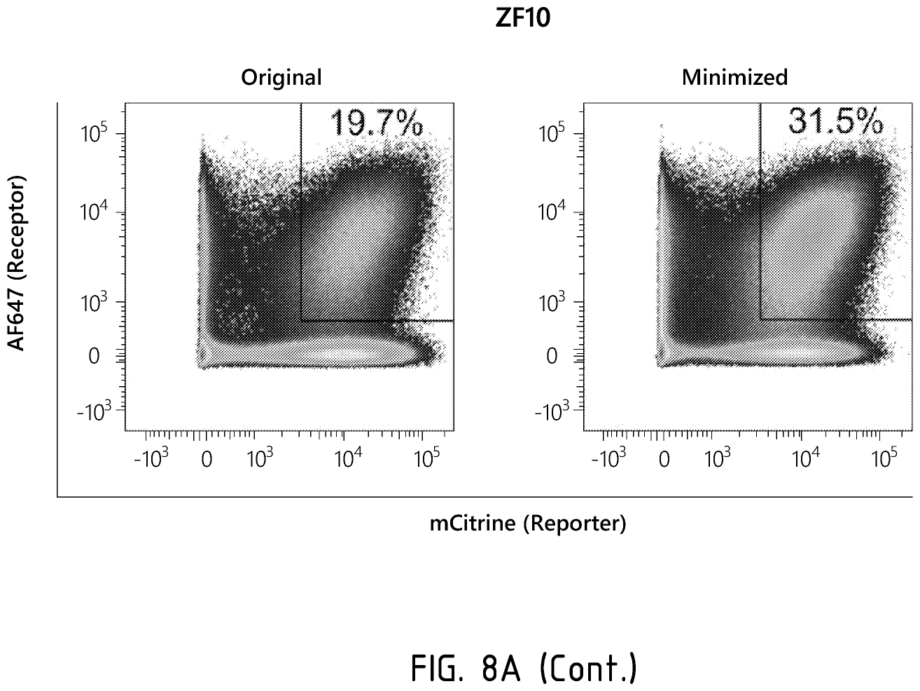

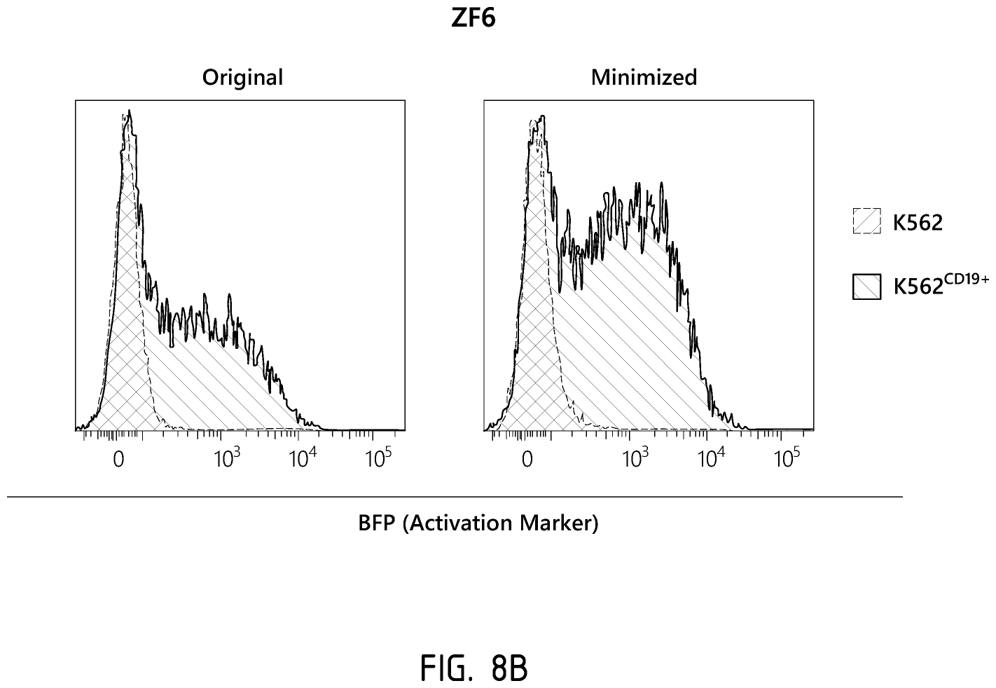

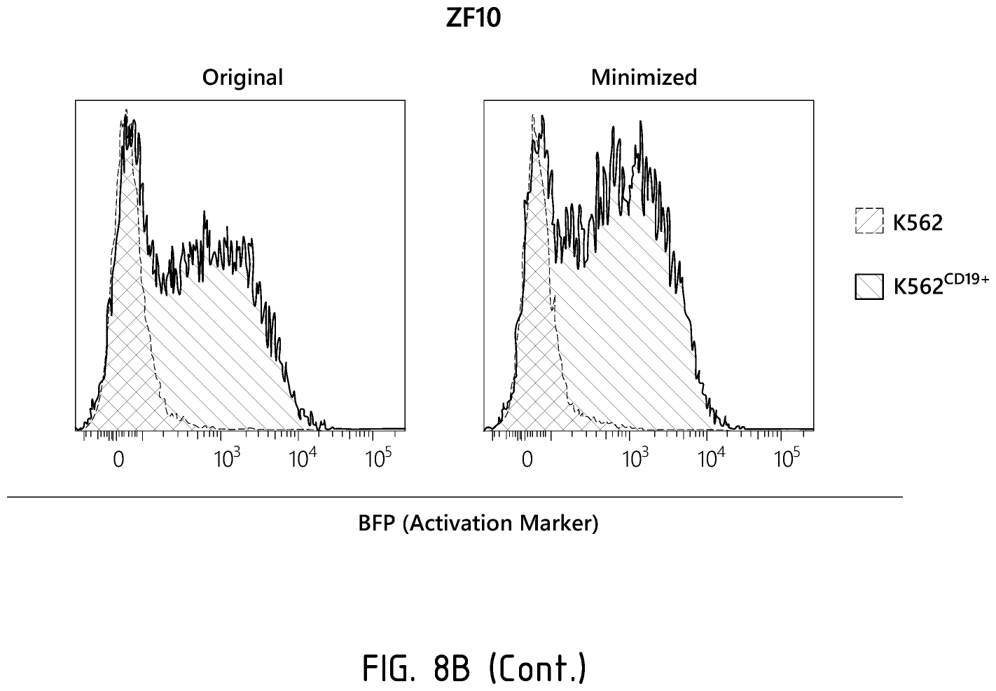

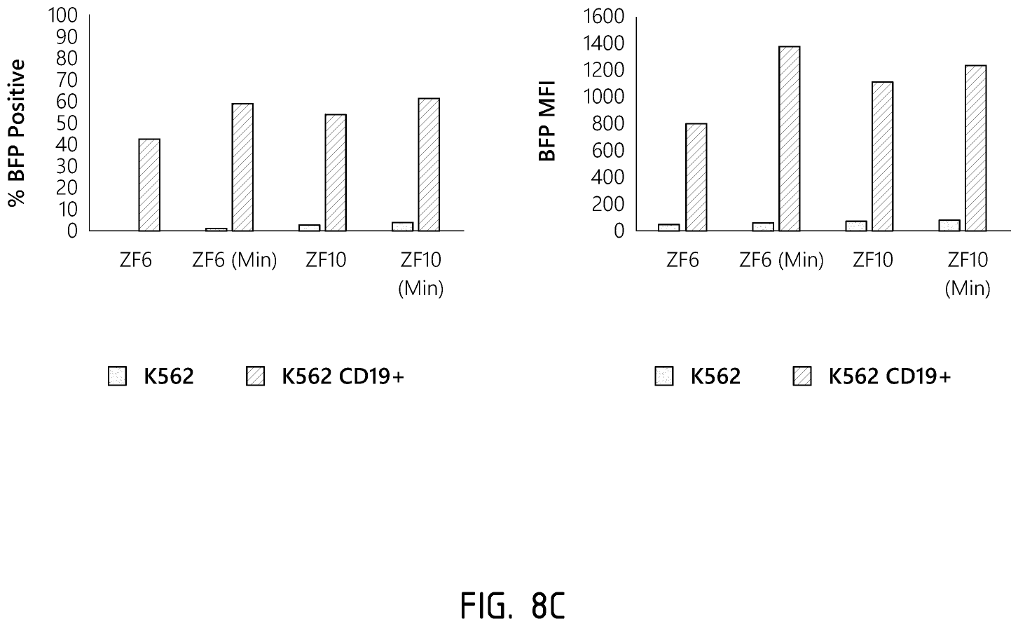

Additional experiments were performed to further evaluate a number of synZTE-containing HingeNotch variants described in FIGS. 7A-7C in CD8+ cells. In these experiments, the expression profiles of original synZTE-containing HingeNotch receptor were compared with the partially minimized synZTE-HingeNotch variants bearing ZF6 or ZF10, as described in FIGS. 7A-7C above. As shown in FIG. 8A, the minimized versions bear none of the linker sequences deleted but retain the full-length transactivation domain p65. Notably, the minimized form of the ZF10-bearing HingeNotch receptor demonstrated improved expression relative to the original full length sequence, although the same effect was not observed in the case of ZF6.

As presented in FIG. 8B, it was observed that the minimized forms of both ZF6 and ZF10-bearing HingeNotch receptors demonstrated augmented activation relative to their original counterparts, as determined by BFP expression from the construct referenced in FIG. 8A after stimulation with unmodified or CD19-expressing K562 cells. In addition, FIG. 8C depicts the percent BFP-expressing T-cells (left panel) and BFP MFI (right panel) tabulated for the data in FIG. 8B.

### Example 11

### Modification of NLS to Modulate Receptor Activity

This Example describes the results of experiments performed to further optimize synZTE-containing HingeNotch receptors by modifying the sequences of NLS.

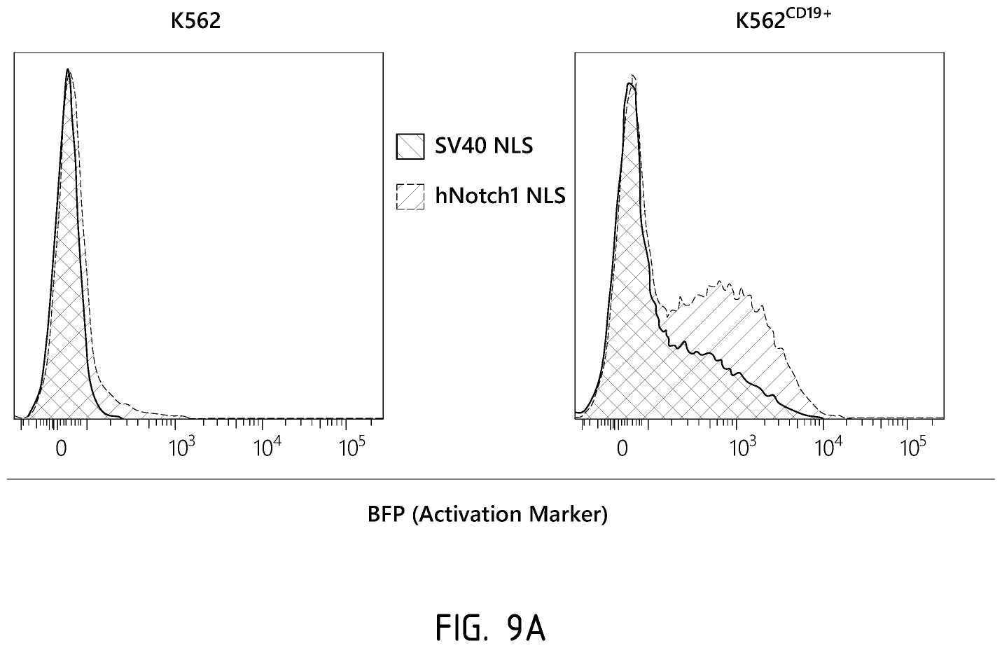

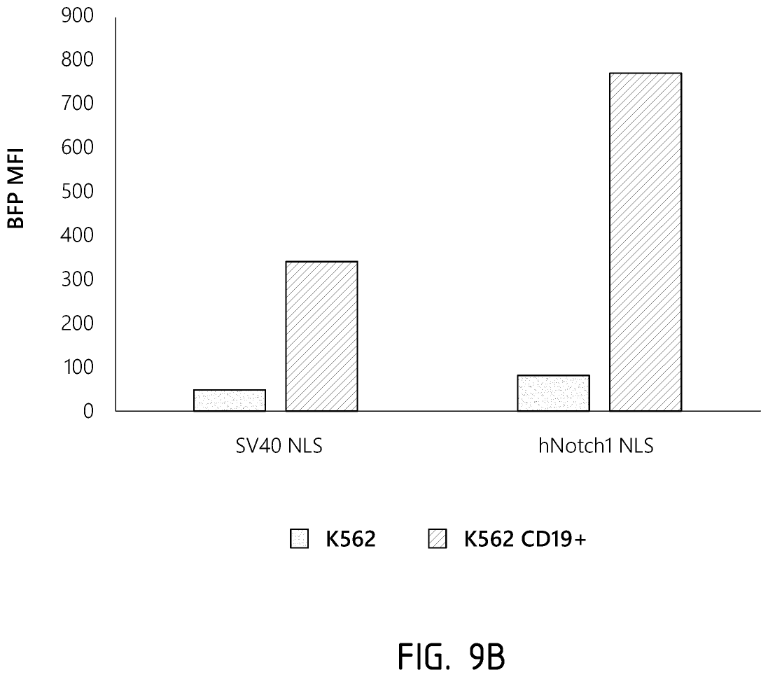

In these experiments, primary CD4+ T-cells were transduced with MiniNotch receptor variants bearing synthetic zinc finger-containing transcriptional activators (SynTFs) consisting of the ZF3 zinc finger and transactivation domain p65, with either the original SV40 NLS or the hNotch1 NLS. BFP expression of the transduced primary CD4+ T-cells is shown in FIG. 9A.

FIG. 9B shows mean fluorescence intensity (MFI) of BFP expression quantified for the experiment shown in FIG. 9A.

### Example 12

### Transcriptional Activation Activity of synZTE-Containing HingeNotch Receptors

This Example describes the results of experiments performed to evaluate the activation performance of synZTE-containing HingeNotch receptors relative to Gal4-VP64.

A number of exemplary synZTE-containing HingeNotch receptors with their respective components are provided in Table 4 below.

TABLE 4
Amino acid sequences of exemplary synZTE-containing HingeNotch receptors
with fused TAD domains.

| | | Seq | |
|---|---|---|---|
| | | ID | |
| Name | Description | NO | Protein Sequence |
| pDP1380 | α-CD19 | 124 | MALPVTALLLPLALLLHAARPEQKLISEEDLDIQMTQTTSSLSASL |
| | HingeNotch | | GDRVTISCRASQDISKYLNWYQQKPDGTVKLLIYHTSRLHSGVPSR |
| | ZF6-p65 | | FSGSGSGTDYSLTISNLEQEDIATYFCQQGNTLPYTFGGGTKLEIT |
| | | | GGGGSGGGGSGGGGSEVKLQESGPGLVAPSQSLSVTCTVSGVSLPD |
| | | | YGVSWIRQPPRKGLEWLGVIWGSETTYYNSALKSRLTIIKDNSKSQ |
| | | | VFLKMNSLQTDDTAIYYCAKHYYYGGSYAMDYWGQGTSVTVSSTTT |
| | | | PAPRPPTPAPTIASQPLSLRPEACFMYVAAAAFVLLFFVGCGVLLS |
| | | | KRKRKHPKKKRKVSRPGERPFQCRICMRNFSQQTNLTRHTRTHTGE |
| | | | KPFQCRICMRNFSQKANLGVHLRTHTGSQKPFQCRICMRNFSDHSS |
| | | | LKRHTRTHTGEKPFQCRICMRNFSRADMLRRHLRTHTGSQKPFQCR |
| | | | ICMRNFSEKQNLARHTRTHTGEKPFQCRICMRNFSDPSNLRRHLRT |
| | | | HLRDEFPTMVFPSGQISQASALAPAPPQVLPQAPAPAPAPAMVSAL |
| | | | AQAPAPVPVLAPGPPQAVAPPAPKPTQAGEGTLSEALLQLQFDDED |
| | | | LGALLGNSTDPAVFTDLASVDNSEFQQLLNQGIPVAPHTTEPMLME |
| | | | YPEAITRLVTGAQRPPDPAPAPLGAPGLPNGLLSGDEDFSSIADMD |
| | | | FSALLSQISS |
| pDP1381 | α-CD19 | 125 | MALPVTALLLPLALLLHAARPEQKLISEEDLDIQMTQTTSSLSASL |
| | HingeNotch | | GDRVTISCRASQDISKYLNWYQQKPDGTVKLLIYHTSRLHSGVPSR |
| | ZF6-VP64 | | FSGSGSGTDYSLTISNLEQEDIATYFCQQGNTLPYTFGGGTKLEIT |
| | | | GGGGSGGGGSGGGGSEVKLQESGPGLVAPSQSLSVTCTVSGVSLPD |
| | | | YGVSWIRQPPRKGLEWLGVIWGSETTYYNSALKSRLTIIKDNSKSQ |
| | | | VFLKMNSLQTDDTAIYYCAKHYYYGGSYAMDYWGQGTSVTVSSTTT |
| | | | PAPRPPTPAPTIASQPLSLRPEACFMYVAAAAFVLLFFVGCGVLLS |
| | | | KRKRKHPKKKRKVSRPGERPFQCRICMRNFSQQTNLTRHTRTHTGE |
| | | | KPFQCRICMRNFSQKANLGVHLRTHTGSQKPFQCRICMRNFSDHSS |
| | | | LKRHTRTHTGEKPFQCRICMRNFSRADMLRRHLRTHTGSQKPFQCR |
| | | | ICMRNFSEKQNLARHTRTHTGEKPFQCRICMRNFSDPSNLRRHLRT |
| | | | HLRDALDDFDLDMLGSDALDDFDLDMLGSDALDDFDLDMLGSDALD |
| | | | DFDLDMLGS |
| pDP1382 | α-CD19 | 126 | MALPVTALLLPLALLLHAARPEQKLISEEDLDIQMTQTTSSLSASL |
| | HingeNotch | | GDRVTISCRASQDISKYLNWYQQKPDGTVKLLIYHTSRLHSGVPSR |
| | ZF10-p65 | | FSGSGSGTDYSLTISNLEQEDIATYFCQQGNTLPYTFGGGTKLEIT |
| | | | GGGGSGGGGSGGGGSEVKLQESGPGLVAPSQSLSVTCTVSGVSLPD |
| | | | YGVSWIRQPPRKGLEWLGVIWGSETTYYNSALKSRLTIIKDNSKSQ |
| | | | VFLKMNSLQTDDTAIYYCAKHYYYGGSYAMDYWGQGTSVTVSSTTT |
| | | | PAPRPPTPAPTIASQPLSLRPEACFMYVAAAAFVLLFFVGCGVLLS |
| | | | KRKRKHPKKKRKVSRPGERPFQCRICMRNFSRRHGLDRHTRTHTGE |
| | | | KPFQCRICMRNFSDHSSLKRHLRTHTGSQKPFQCRICMRNFSVRHN |
| | | | LTRHLRTHTGEKPFQCRICMRNFSDHSNLSRHLKTHTGSQKPFQCR |
| | | | ICMRNFSQRSSLVRHLRTHTGEKPFQCRICMRNFSESGHLKRHLRT |
| | | | HLRDEFPTMVFPSGQISQASALAPAPPQVLPQAPAPAPAPAMVSAL |
| | | | AQAPAPVPVLAPGPPQAVAPPAPKPTQAGEGTLSEALLQLQFDDED |
| | | | LGALLGNSTDPAVFTDLASVDNSEFQQLLNQGIPVAPHTTEPMLME |
| | | | YPEAITRLVTGAQRPPDPAPAPLGAPGLPNGLLSGDEDFSSIADMD |
| | | | FSALLSQISS |
| pDP1383 | α-CD19 | 127 | MALPVTALLLPLALLLHAARPEQKLISEEDLDIQMTQTTSSLSASL |
| | HingeNotch | | GDRVTISCRASQDISKYLNWYQQKPDGTVKLLIYHTSRLHSGVPSR |
| | ZF10-VP64 | | FSGSGSGTDYSLTISNLEQEDIATYFCQQGNTLPYTFGGGTKLEIT |
| | | | GGGGSGGGGSGGGGSEVKLQESGPGLVAPSQSLSVTCTVSGVSLPD |
| | | | YGVSWIRQPPRKGLEWLGVIWGSETTYYNSALKSRLTIIKDNSKSQ |
| | | | VFLKMNSLQTDDTAIYYCAKHYYYGGSYAMDYWGQGTSVTVSSTTT |
| | | | PAPRPPTPAPTIASQPLSLRPEACFMYVAAAAFVLLFFVGCGVLLS |
| | | | KRKRKHPKKKRKVSRPGERPFQCRICMRNFSRRHGLDRHTRTHTGE |
| | | | KPFQCRICMRNFSDHSSLKRHLRTHTGSQKPFQCRICMRNFSVRHN |
| | | | LTRHLRTHTGEKPFQCRICMRNFSDHSNLSRHLKTHTGSQKPFQCR |
| | | | ICMRNFSQRSSLVRHLRTHTGEKPFQCRICMRNFSESGHLKRHLRT |
| | | | HLRDALDDFDLDMLGSDALDDFDLDMLGSDALDDFDLDMLGSDALD |
| | | | DFDLDMLGS |
| pDP1384 | α-CD19 | 128 | MALPVTALLLPLALLLHAARPEQKLISEEDLDIQMTQTTSSLSASL |
| | HingeNotch | | GDRVTISCRASQDISKYLNWYQQKPDGTVKLLIYHTSRLHSGVPSR |
| | Gal4-p65 | | FSGSGSGTDYSLTISNLEQEDIATYFCQQGNTLPYTFGGGTKLEIT |
| | | | GGGGSGGGGSGGGGSEVKLQESGPGLVAPSQSLSVTCTVSGVSLPD |
| | | | YGVSWIRQPPRKGLEWLGVIWGSETTYYNSALKSRLTIIKDNSKSQ |
| | | | VFLKMNSLQTDDTAIYYCAKHYYYGGSYAMDYWGQGTSVTVSSTTT |
| | | | PAPRPPTPAPTIASQPLSLRPEACFMYVAAAAFVLLFFVGCGVLLS |
| | | | KRKRKHMKLLSSIEQACDICRLKKLKCSKEKPKCAKCLKNNWECRY |
| | | | SPKTKRSPLTRAHLTEVESRLERLEQLFLLIFPREDLDMILKMDSL |
| | | | QDIKALLTGLFVQDNVNKDAVTDRLASVETDMPLTLRQHRISATSS |
| | | | SEESSNKGQRQLTVSAAAGGSGGSGGSDEFPTMVFPSGQISQASAL |
| | | | APAPPQVLPQAPAPAPAPAMVSALAQAPAPVPVLAPGPPQAVAPPA |
| | | | PKPTQAGEGTLSEALLQLQFDDEDLGALLGNSTDPAVFTDLASVDN |
| | | | SEFQQLLNQGIPVAPHTTEPMLMEYPEAITRLVTGAQRPPDPAPAP |
| | | | LGAPGLPNGLLSGDEDFSSIADMDFSALLSQISS |
| pDP1385 | α-CD19 | 129 | MALPVTALLLPLALLLHAARPEQKLISEEDLDIQMTQTTSSLSASL |
| | HingeNotch | | GDRVTISCRASQDISKYLNWYQQKPDGTVKLLIYHTSRLHSGVPSR |
| | Gal4-VP64 | | FSGSGSGTDYSLTISNLEQEDIATYFCQQGNTLPYTFGGGTKLEIT |
| | | | GGGGSGGGGSGGGGSEVKLQESGPGLVAPSQSLSVTCTVSGVSLPD |
| | | | YGVSWIRQPPRKGLEWLGVIWGSETTYYNSALKSRLTIIKDNSKSQ |
| | | | VFLKMNSLQTDDTAIYYCAKHYYYGGSYAMDYWGQGTSVTVSSTTT |
| | | | PAPRPPTPAPTIASQPLSLRPEACFMYVAAAAFVLLFFVGCGVLLS |
| | | | KRKRKHMKLLSSIEQACDICRLKKLKCSKEKPKCAKCLKNNWECRY |
| | | | SPKTKRSPLTRAHLTEVESRLERLEQLFLLIFPREDLDMILKMDSL |
| | | | QDIKALLTGLFVQDNVNKDAVTDRLASVETDMPLTLRQHRISATSS |
| | | | SEESSNKGQRQLTVSAAAGGSGGSGGSDALDDFDLDMLGSDALDDF |
| | | | DLDMLGSDALDDFDLDMLGSDALDDFDLDMLGS |
| pDP1386 | α-CD19 | 130 | MALPVTALLLPLALLLHAARPEQKLISEEDLDIQMTQTTSSLSASL |
| | HingeNotch | | GDRVTISCRASQDISKYLNWYQQKPDGTVKLLIYHTSRLHSGVPSR |
| | ZF6-p65- | | FSGSGSGTDYSLTISNLEQEDIATYFCQQGNTLPYTFGGGTKLEIT |
| | HSF1 | | GGGGSGGGGSGGGGSEVKLQESGPGLVAPSQSLSVTCTVSGVSLPD |
| | | | YGVSWIRQPPRKGLEWLGVIWGSETTYYNSALKSRLTIIKDNSKSQ |
| | | | VFLKMNSLQTDDTAIYYCAKHYYYGGSYAMDYWGQGTSVTVSSTTT |
| | | | PAPRPPTPAPTIASQPLSLRPEACFMYVAAAAFVLLFFVGCGVLLS |
| | | | KRKRKHPKKKRKVSRPGERPFQCRICMRNFSQQTNLTRHTRTHTGE |
| | | | KPFQCRICMRNFSQKANLGVHLRTHTGSQKPFQCRICMRNFSDHSS |
| | | | LKRHTRTHTGEKPFQCRICMRNFSRADMLRRHLRTHTGSQKPFQCR |
| | | | ICMRNFSEKQNLARHTRTHTGEKPFQCRICMRNFSDPSNLRRHLRT |
| | | | HLRDEFPTMVFPSGQISQASALAPAPPQVLPQAPAPAPAPAMVSAL |
| | | | AQAPAPVPVLAPGPPQAVAPPAPKPTQAGEGTLSEALLQLQFDDED |
| | | | LGALLGNSTDPAVFTDLASVDNSEFQQLLNQGIPVAPHTTEPMLME |
| | | | YPEAITRLVTGAQRPPDPAPAPLGAPGLPNGLLSGDEDFSSIADMD |
| | | | FSALLSQISSSGQGGGGSGFSVDTSALLDLFSPSVTVPDMSLPDLD |
| | | | SSLASIQELLSPQEPPRPPEAENSSPDSGKQLVHYTAQPLFLLDPG |
| | | | SVDTGSNDLPVLFELGEGSYFSEGDGFAEDPTISLLTGSEPPKAKD |
| | | | PTVS |
| pDP1387 | α-CD19 | 131 | MALPVTALLLPLALLLHAARPEQKLISEEDLDIQMTQTTSSLSASL |
| | HingeNotch | | GDRVTISCRASQDISKYLNWYQQKPDGTVKLLIYHTSRLHSGVPSR |
| | ZF6-p65- | | FSGSGSGTDYSLTISNLEQEDIATYFCQQGNTLPYTFGGGTKLEIT |
| | GR1 | | GGGGSGGGGSGGGGSEVKLQESGPGLVAPSQSLSVTCTVSGVSLPD |
| | | | YGVSWIRQPPRKGLEWLGVIWGSETTYYNSALKSRLTIIKDNSKSQ |
| | | | VFLKMNSLQTDDTAIYYCAKHYYYGGSYAMDYWGQGTSVTVSSTTT |
| | | | PAPRPPTPAPTIASQPLSLRPEACFMYVAAAAFVLLFFVGCGVLLS |
| | | | KRKRKHPKKKRKVSRPGERPFQCRICMRNFSQQTNLTRHTRTHTGE |
| | | | KPFQCRICMRNFSQKANLGVHLRTHTGSQKPFQCRICMRNFSDHSS |
| | | | LKRHTRTHTGEKPFQCRICMRNFSRADMLRRHLRTHTGSQKPFQCR |
| | | | ICMRNFSEKQNLARHTRTHTGEKPFQCRICMRNFSDPSNLRRHLRT |
| | | | HLRDEFPTMVFPSGQISQASALAPAPPQVLPQAPAPAPAPAMVSAL |
| | | | AQAPAPVPVLAPGPPQAVAPPAPKPTQAGEGTLSEALLQLQFDDED |
| | | | LGALLGNSTDPAVFTDLASVDNSEFQQLLNQGIPVAPHTTEPMLME |
| | | | YPEAITRLVTGAQRPPDPAPAPLGAPGLPNGLLSGDEDFSSIADMD |
| | | | FSALLSQISSSGQGGGGSDQSTFDILQDLEFSSGSPGKETNESPWR |
| | | | SDLLIDENCLLSPLAGEDDSFLLEGNSNED |
| pDP1388 | α-CD19 | 132 | MALPVTALLLPLALLLHAARPEQKLISEEDLDIQMTQTTSSLSASL |
| | HingeNotch | | GDRVTISCRASQDISKYLNWYQQKPDGTVKLLIYHTSRLHSGVPSR |
| | ZF10-p65- | | FSGSGSGTDYSLTISNLEQEDIATYFCQQGNTLPYTFGGGTKLEIT |
| | HSF1 | | GGGGSGGGGSGGGGSEVKLQESGPGLVAPSQSLSVTCTVSGVSLPD |
| | | | YGVSWIRQPPRKGLEWLGVIWGSETTYYNSALKSRLTIIKDNSKSQ |
| | | | VFLKMNSLQTDDTAIYYCAKHYYYGGSYAMDYWGQGTSVTVSSTTT |
| | | | PAPRPPTPAPTIASQPLSLRPEACFMYVAAAAFVLLFFVGCGVLLS |
| | | | KRKRKHPKKKRKVSRPGERPFQCRICMRNFSRRHGLDRHTRTHTGE |
| | | | KPFQCRICMRNFSDHSSLKRHLRTHTGSQKPFQCRICMRNFSVRHN |
| | | | LTRHLRTHTGEKPFQCRICMRNFSDHSNLSRHLKTHTGSQKPFQCR |
| | | | ICMRNFSQRSSLVRHLRTHTGEKPFQCRICMRNFSESGHLKRHLRT |
| | | | HLRDEFPTMVFPSGQISQASALAPAPPQVLPQAPAPAPAPAMVSAL |
| | | | AQAPAPVPVLAPGPPQAVAPPAPKPTQAGEGTLSEALLQLQFDDED |
| | | | LGALLGNSTDPAVFTDLASVDNSEFQQLLNQGIPVAPHTTEPMLME |
| | | | YPEAITRLVTGAQRPPDPAPAPLGAPGLPNGLLSGDEDFSSIADMD |
| | | | FSALLSQISSSGQGGGGSGFSVDTSALLDLFSPSVTVPDMSLPDLD |
| | | | SSLASIQELLSPQEPPRPPEAENSSPDSGKQLVHYTAQPLFLLDPG |
| | | | SVDTGSNDLPVLFELGEGSYFSEGDGFAEDPTISLLTGSEPPKAKD |
| | | | PTVS |
| pDP1389 | α-CD19 | 133 | MALPVTALLLPLALLLHAARPEQKLISEEDLDIQMTQTTSSLSASL |
| | HingeNotch | | GDRVTISCRASQDISKYLNWYQQKPDGTVKLLIYHTSRLHSGVPSR |
| | ZF10-p65- | | FSGSGSGTDYSLTISNLEQEDIATYFCQQGNTLPYTFGGGTKLEIT |
| | GR1 | | GGGGSGGGGSGGGGSEVKLQESGPGLVAPSQSLSVTCTVSGVSLPD |
| | | | YGVSWIRQPPRKGLEWLGVIWGSETTYYNSALKSRLTIIKDNSKSQ |
| | | | VFLKMNSLQTDDTAIYYCAKHYYYGGSYAMDYWGQGTSVTVSSTTT |
| | | | PAPRPPTPAPTIASQPLSLRPEACFMYVAAAAFVLLFFVGCGVLLS |
| | | | KRKRKHPKKKRKVSRPGERPFQCRICMRNFSRRHGLDRHTRTHTGE |
| | | | KPFQCRICMRNFSDHSSLKRHLRTHTGSQKPFQCRICMRNFSVRHN |
| | | | LTRHLRTHTGEKPFQCRICMRNFSDHSNLSRHLKTHTGSQKPFQCR |
| | | | ICMRNFSQRSSLVRHLRTHTGEKPFQCRICMRNFSESGHLKRHLRT |
| | | | HLRDEFPTMVFPSGQISQASALAPAPPQVLPQAPAPAPAPAMVSAL |
| | | | AQAPAPVPVLAPGPPQAVAPPAPKPTQAGEGTLSEALLQLQFDDED |
| | | | LGALLGNSTDPAVFTDLASVDNSEFQQLLNQGIPVAPHTTEPMLME |
| | | | YPEAITRLVTGAQRPPDPAPAPLGAPGLPNGLLSGDEDFSSIADMD |
| | | | FSALLSQISSSGQGGGGSDQSTFDILQDLEFSSGSPGKETNESPWR |
| | | | SDLLIDENCLLSPLAGEDDSFLLEGNSNED |

In these experiments, primary human CD4+ T-cells were transduced with α-CD19 HingeNotch receptors bearing Zinc Finger or Gal4 DNA binding domains fused to one of several transactivation domains. T-cells were co-incubated for 48 hours with K562 cells (unmodified or expressing the cognate CD19 antigen). T-cells were co-transduced with a reporter vector encoding BFP expressed by a promoter cognate to the DNA binding domain of the receptor (ZF6, ZF10, or Gal4). Fluorescence was assayed via flow cytometry. As shown in FIG. 10, the augmented transactivation domain fusions (p65-GR1 and p65-HSF1) enable HingeNotch receptors bearing zinc finger DNA binding domains to activate to an output level similar to that of the original Gal4-VP64-containing receptor, with protein expression approximately 5-7 fold higher than with a transactivation domain consisting solely of the p65 TAD.

### Example 13

### Specific Target Cell Killing Via synZTE-Containing HingeNotch Receptors

This Example describes the specific target cell killing via synZTE-containing HingeNotch receptors.

The exemplary synZTE-containing HingeNotch receptors with their respective components are provided in Table 5 below.

TABLE 5
Amino acid sequences of exemplary synZTE-containing HingeNotch receptors
with fused TAD domains used in this Example.

| | | Seq | |
|---|---|---|---|
| Name | Description | ID NO | Protein Sequence |
| pDP1338 | α-ALPPL2 | 134 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF6 | | FTISRDNSKNTLYLQMDSLRAEDTAVYYCAKEGDSSRWSYDLWGRGTLV |
| | p65 | | TVSSGGGGSGGGGSGGGGSQSALTQPASVSGSPGQSITISCTGTSSDVG |
| | | | GYNYVSWYQQHPGKAPKVMIYDVTNRPSGVSNRFSGSKSGNTASLTISG |
| | | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHPKKKRKVSRPG |
| | | | ERPFQCRICMRNFSQQTNLTRHTRTHTGEKPFQCRICMRNFSQKANLGV |
| | | | HLRTHTGSQKPFQCRICMRNFSDHSSLKRHTRTHTGEKPFQCRICMRNF |
| | | | SRADMLRRHLRTHTGSQKPFQCRICMRNFSEKQNLARHTRTHTGEKPFQ |
| | | | CRICMRNFSDPSNLRRHLRTHLRDEFPTMVFPSGQISQASALAPAPPQV |
| | | | LPQAPAPAPAPAMVSALAQAPAPVPVLAPGPPQAVAPPAPKPTQAGEGT |
| | | | LSEALLQLQFDDEDLGALLGNSTDPAVFTDLASVDNSEFQQLLNQGIPV |
| | | | APHTTEPMLMEYPEAITRLVTGAQRPPDPAPAPLGAPGLPNGLLSGDED |
| | | | FSSIADMDFSALLSQISS |
| pDP1339 | α-ALPPL2 | 135 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF6 | | FTISRDNSKNTLYLQMDSLRAEDTAVYYCAKEGDSSRWSYDLWGRGTLV |
| | p65 | | TVSSGGGGSGGGGSGGGGSQSALTQPASVSGSPGQSITISCTGTSSDVG |
| | HSF1 | | GYNYVSWYQQHPGKAPKVMIYDVTNRPSGVSNRFSGSKSGNTASLTISG |
| | | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHPKKKRKVSRPG |
| | | | ERPFQCRICMRNFSQQTNLTRHTRTHTGEKPFQCRICMRNFSQKANLGV |
| | | | HLRTHTGSQKPFQCRICMRNFSDHSSLKRHTRTHTGEKPFQCRICMRNF |
| | | | SRADMLRRHLRTHTGSQKPFQCRICMRNFSEKQNLARHTRTHTGEKPFQ |
| | | | CRICMRNFSDPSNLRRHLRTHLRDEFPTMVFPSGQISQASALAPAPPQV |
| | | | LPQAPAPAPAPAMVSALAQAPAPVPVLAPGPPQAVAPPAPKPTQAGEGT |
| | | | LSEALLQLQFDDEDLGALLGNSTDPAVFTDLASVDNSEFQQLLNQGIPV |
| | | | APHTTEPMLMEYPEAITRLVTGAQRPPDPAPAPLGAPGLPNGLLSGDED |
| | | | FSSIADMDFSALLSQISSSGQGGGGSGFSVDTSALLDLFSPSVTVPDMS |
| | | | LPDLDSSLASIQELLSPQEPPRPPEAENSSPDSGKQLVHYTAQPLFLLD |
| | | | PGSVDTGSNDLPVLFELGEGSYFSEGDGFAEDPTISLLTGSEPPKAKDP |
| | | | TVS |
| pDP1340 | α-ALPPL2 | 136 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF6 | | FTISRDNSKNTLYLQMDSLRAEDTAVYYCAKEGDSSRWSYDLWGRGTLV |
| | p65 GR1 | | TVSSGGGGSGGGGSGGGGSQSALTQPASVSGSPGQSITISCTGTSSDVG |
| | | | GYNYVSWYQQHPGKAPKVMIYDVTNRPSGVSNRFSGSKSGNTASLTISG |
| | | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHPKKKRKVSRPG |
| | | | ERPFQCRICMRNFSQQTNLTRHTRTHTGEKPFQCRICMRNFSQKANLGV |
| | | | HLRTHTGSQKPFQCRICMRNFSDHSSLKRHTRTHTGEKPFQCRICMRNF |
| | | | SRADMLRRHLRTHTGSQKPFQCRICMRNFSEKQNLARHTRTHTGEKPFQ |
| | | | CRICMRNFSDPSNLRRHLRTHLRDEFPTMVFPSGQISQASALAPAPPQV |
| | | | LPQAPAPAPAPAMVSALAQAPAPVPVLAPGPPQAVAPPAPKPTQAGEGT |
| | | | LSEALLQLQFDDEDLGALLGNSTDPAVFTDLASVDNSEFQQLLNQGIPV |
| | | | APHTTEPMLMEYPEAITRLVTGAQRPPDPAPAPLGAPGLPNGLLSGDED |
| | | | FSSIADMDFSALLSQISSSGQGGGGSDQSTFDILQDLEFSSGSPGKETN |
| | | | ESPWRSDLLIDENCLLSPLAGEDDSFLLEGNSNED |
| pDP1341 | α-ALPPL2 | 137 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF6 | | FTISRDNSKNTLYLQMDSLRAEDTAVYYCAKEGDSSRWSYDLWGRGTLV |
| | p65 | | TVSSGGGGSGGGGSGGGGSQSALTQPASVSGSPGQSITISCTGTSSDVG |
| | Notch1NLS | | GYNYVSWYQQHPGKAPKVMIYDVTNRPSGVSNRFSGSKSGNTASLTISG |
| | | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHRRKKSRPGERP |
| | | | FQCRICMRNFSQQTNLTRHTRTHTGEKPFQCRICMRNFSQKANLGVHLR |
| | | | THTGSQKPFQCRICMRNFSDHSSLKRHTRTHTGEKPFQCRICMRNFSRA |
| | | | DMLRRHLRTHTGSQKPFQCRICMRNFSEKQNLARHTRTHTGEKPFQCRI |
| | | | CMRNFSDPSNLRRHLRTHLRDEFPTMVFPSGQISQASALAPAPPQVLPQ |
| | | | APAPAPAPAMVSALAQAPAPVPVLAPGPPQAVAPPAPKPTQAGEGTLSE |
| | | | ALLQLQFDDEDLGALLGNSTDPAVFTDLASVDNSEFQQLLNQGIPVAPH |
| | | | TTEPMLMEYPEAITRLVTGAQRPPDPAPAPLGAPGLPNGLLSGDEDFSS |
| | | | IADMDFSALLSQISS |
| pDP1342 | α-ALPPL2 | 138 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF6 | | FTISRDNSKNTLYLQMDSLRAEDTAVYYCAKEGDSSRWSYDLWGRGTLV |
| | p65 | | TVSSGGGGSGGGGSGGGGSQSALTQPASVSGSPGQSITISCTGTSSDVG |
| | HSF1 | | GYNYVSWYQQHPGKAPKVMIYDVTNRPSGVSNRFSGSKSGNTASLTISG |
| | Notch1NLS | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHRRKKSRPGERP |
| | | | FQCRICMRNFSQQTNLTRHTRTHTGEKPFQCRICMRNFSQKANLGVHLR |
| | | | THTGSQKPFQCRICMRNFSDHSSLKRHTRTHTGEKPFQCRICMRNFSRA |
| | | | DMLRRHLRTHTGSQKPFQCRICMRNFSEKQNLARHTRTHTGEKPFQCRI |
| | | | CMRNFSDPSNLRRHLRTHLRDEFPTMVFPSGQISQASALAPAPPQVLPQ |
| | | | APAPAPAPAMVSALAQAPAPVPVLAPGPPQAVAPPAPKPTQAGEGTLSE |
| | | | ALLQLQFDDEDLGALLGNSTDPAVFTDLASVDNSEFQQLLNQGIPVAPH |
| | | | TTEPMLMEYPEAITRLVTGAQRPPDPAPAPLGAPGLPNGLLSGDEDFSS |
| | | | IADMDFSALLSQISSSGQGGGGSGFSVDTSALLDLFSPSVTVPDMSLPD |
| | | | LDSSLASIQELLSPQEPPRPPEAENSSPDSGKQLVHYTAQPLFLLDPGS |
| | | | VDTGSNDLPVLFELGEGSYFSEGDGFAEDPTISLLTGSEPPKAKDPTVS |
| pDP1343 | α-ALPPL2 | 139 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF6 | | FTISRDNSKNTLYLQMDSLRAEDTAVYYCAKEGDSSRWSYDLWGRGTLV |
| | p65 GR1 | | TVSSGGGGSGGGGSGGGGSQSALTQPASVSGSPGQSITISCTGTSSDVG |
| | Notch1NLS | | GYNYVSWYQQHPGKAPKVMIYDVTNRPSGVSNRFSGSKSGNTASLTISG |
| | | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHRRKKSRPGERP |
| | | | FQCRICMRNFSQQTNLTRHTRTHTGEKPFQCRICMRNFSQKANLGVHLR |
| | | | THTGSQKPFQCRICMRNFSDHSSLKRHTRTHTGEKPFQCRICMRNFSRA |
| | | | DMLRRHLRTHTGSQKPFQCRICMRNFSEKQNLARHTRTHTGEKPFQCRI |
| | | | CMRNFSDPSNLRRHLRTHLRDEFPTMVFPSGQISQASALAPAPPQVLPQ |
| | | | APAPAPAPAMVSALAQAPAPVPVLAPGPPQAVAPPAPKPTQAGEGTLSE |
| | | | ALLQLQFDDEDLGALLGNSTDPAVFTDLASVDNSEFQQLLNQGIPVAPH |
| | | | TTEPMLMEYPEAITRLVTGAQRPPDPAPAPLGAPGLPNGLLSGDEDFSS |
| | | | IADMDFSALLSQISSSGQGGGGSDQSTFDILQDLEFSSGSPGKETNESP |
| | | | WRSDLLIDENCLLSPLAGEDDSFLLEGNSNED |
| pDP1344 | α-ALPPL2 | 140 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF10 | | FTISRDNSKNTLYLQMDSLRAEDTAVYYCAKEGDSSRWSYDLWGRGTLV |
| | p65 | | TVSSGGGGSGGGGSGGGGSQSALTQPASVSGSPGQSITISCTGTSSDVG |
| | | | GYNYVSWYQQHPGKAPKVMIYDVTNRPSGVSNRFSGSKSGNTASLTISG |
| | | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHPKKKRKVSRPG |
| | | | ERPFQCRICMRNFSRRHGLDRHTRTHTGEKPFQCRICMRNFSDHSSLKR |
| | | | HLRTHTGSQKPFQCRICMRNFSVRHNLTRHLRTHTGEKPFQCRICMRNF |
| | | | SDHSNLSRHLKTHTGSQKPFQCRICMRNFSQRSSLVRHLRTHTGEKPFQ |
| | | | CRICMRNFSESGHLKRHLRTHLRDEFPTMVFPSGQISQASALAPAPPQV |
| | | | LPQAPAPAPAPAMVSALAQAPAPVPVLAPGPPQAVAPPAPKPTQAGEGT |
| | | | LSEALLQLQFDDEDLGALLGNSTDPAVFTDLASVDNSEFQQLLNQGIPV |
| | | | APHTTEPMLMEYPEAITRLVTGAQRPPDPAPAPLGAPGLPNGLLSGDED |
| | | | FSSIADMDFSALLSQISS |
| pDP1345 | α-ALPPL2 | 141 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF10 | | FTISRDNSKNTLYLQMDSLRAEDTAVYYCAKEGDSSRWSYDLWGRGTLV |
| | p65 | | TVSSGGGGSGGGGSGGGGSQSALTQPASVSGSPGQSITISCTGTSSDVG |
| | HSF1 | | GYNYVSWYQQHPGKAPKVMIYDVTNRPSGVSNRFSGSKSGNTASLTISG |
| | | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHPKKKRKVSRPG |
| | | | ERPFQCRICMRNFSRRHGLDRHTRTHTGEKPFQCRICMRNFSDHSSLKR |
| | | | HLRTHTGSQKPFQCRICMRNFSVRHNLTRHLRTHTGEKPFQCRICMRNF |
| | | | SDHSNLSRHLKTHTGSQKPFQCRICMRNFSQRSSLVRHLRTHTGEKPFQ |
| | | | CRICMRNFSESGHLKRHLRTHLRDEFPTMVFPSGQISQASALAPAPPQV |
| | | | LPQAPAPAPAPAMVSALAQAPAPVPVLAPGPPQAVAPPAPKPTQAGEGT |
| | | | LSEALLQLQFDDEDLGALLGNSTDPAVFTDLASVDNSEFQQLLNQGIPV |
| | | | APHTTEPMLMEYPEAITRLVTGAQRPPDPAPAPLGAPGLPNGLLSGDED |
| | | | FSSIADMDFSALLSQISSSGQGGGGSGFSVDTSALLDLFSPSVTVPDMS |
| | | | LPDLDSSLASIQELLSPQEPPRPPEAENSSPDSGKQLVHYTAQPLFLLD |
| | | | PGSVDTGSNDLPVLFELGEGSYFSEGDGFAEDPTISLLTGSEPPKAKDP |
| | | | TVS |
| pDP1346 | α-ALPPL2 | 142 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF10 | | FTISRDNSKNTLYLQMDSLRAEDTAVYYCAKEGDSSRWSYDLWGRGTLV |
| | p65 GR1 | | TVSSGGGGSGGGGSGGGGSQSALTQPASVSGSPGQSITISCTGTSSDVG |
| | | | GYNYVSWYQQHPGKAPKVMIYDVTNRPSGVSNRFSGSKSGNTASLTISG |
| | | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHPKKKRKVSRPG |
| | | | ERPFQCRICMRNFSRRHGLDRHTRTHTGEKPFQCRICMRNFSDHSSLKR |
| | | | HLRTHTGSQKPFQCRICMRNFSVRHNLTRHLRTHTGEKPFQCRICMRNF |
| | | | SDHSNLSRHLKTHTGSQKPFQCRICMRNFSQRSSLVRHLRTHTGEKPFQ |
| | | | CRICMRNFSESGHLKRHLRTHLRDEFPTMVFPSGQISQASALAPAPPQV |
| | | | LPQAPAPAPAPAMVSALAQAPAPVPVLAPGPPQAVAPPAPKPTQAGEGT |
| | | | LSEALLQLQFDDEDLGALLGNSTDPAVFTDLASVDNSEFQQLLNQGIPV |
| | | | APHTTEPMLMEYPEAITRLVTGAQRPPDPAPAPLGAPGLPNGLLSGDED |
| | | | FSSIADMDFSALLSQISSSGQGGGGSDQSTFDILQDLEFSSGSPGKETN |
| | | | ESPWRSDLLIDENCLLSPLAGEDDSFLLEGNSNED |
| pDP1347 | α-ALPPL2 | 143 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF10 | | FTISRDNSKNTLYLQMDSLRAEDTAVYYCAKEGDSSRWSYDLWGRGTLV |
| | p65 | | TVSSGGGGSGGGGSGGGGSQSALTQPASVSGSPGQSITISCTGTSSDVG |
| | Notch1NLS | | GYNYVSWYQQHPGKAPKVMIYDVTNRPSGVSNRFSGSKSGNTASLTISG |
| | | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHRRKKSRPGERP |
| | | | FQCRICMRNFSRRHGLDRHTRTHTGEKPFQCRICMRNFSDHSSLKRHLR |
| | | | THTGSQKPFQCRICMRNFSVRHNLTRHLRTHTGEKPFQCRICMRNFSDH |
| | | | SNLSRHLKTHTGSQKPFQCRICMRNFSQRSSLVRHLRTHTGEKPFQCRI |
| | | | CMRNFSESGHLKRHLRTHLRDEFPTMVFPSGQISQASALAPAPPQVLPQ |
| | | | APAPAPAPAMVSALAQAPAPVPVLAPGPPQAVAPPAPKPTQAGEGTLSE |
| | | | ALLQLQFDDEDLGALLGNSTDPAVFTDLASVDNSEFQQLLNQGIPVAPH |
| | | | TTEPMLMEYPEAITRLVTGAQRPPDPAPAPLGAPGLPNGLLSGDEDFSS |
| | | | IADMDFSALLSQISS |
| pDP1348 | α-ALPPL2 | 144 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF10 | | FTISRDNSKNTLYLQMDSLRAEDTAVYYCAKEGDSSRWSYDLWGRGTLV |
| | p65 | | TVSSGGGGSGGGGSGGGGSQSALTQPASVSGSPGQSITISCTGTSSDVG |
| | HSF1 | | GYNYVSWYQQHPGKAPKVMIYDVTNRPSGVSNRFSGSKSGNTASLTISG |
| | Notch1NLS | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHRRKKSRPGERP |
| | | | FQCRICMRNFSRRHGLDRHTRTHTGEKPFQCRICMRNFSDHSSLKRHLR |
| | | | THTGSQKPFQCRICMRNFSVRHNLTRHLRTHTGEKPFQCRICMRNFSDH |
| | | | SNLSRHLKTHTGSQKPFQCRICMRNFSQRSSLVRHLRTHTGEKPFQCRI |
| | | | CMRNFSESGHLKRHLRTHLRDEFPTMVFPSGQISQASALAPAPPQVLPQ |
| | | | APAPAPAPAMVSALAQAPAPVPVLAPGPPQAVAPPAPKPTQAGEGTLSE |
| | | | ALLQLQFDDEDLGALLGNSTDPAVFTDLASVDNSEFQQLLNQGIPVAPH |
| | | | TTEPMLMEYPEAITRLVTGAQRPPDPAPAPLGAPGLPNGLLSGDEDFSS |
| | | | IADMDFSALLSQISSSGQGGGGSGFSVDTSALLDLFSPSVTVPDMSLPD |
| | | | LDSSLASIQELLSPQEPPRPPEAENSSPDSGKQLVHYTAQPLFLLDPGS |
| | | | VDTGSNDLPVLFELGEGSYFSEGDGFAEDPTISLLTGSEPPKAKDPTVS |
| pDP1349 | α-ALPPL2 | 145 | MALPVTALLLPLALLLHAARPEQKLISEEDLQVQLQQSGGGLVKPGGSL |
| | HingeNotch | | RLSCAASGFTFSSYAMHWVRQAPGKGLEWVAVISYDGSNKYYADSVKGR |
| | ZF10 | | FTISRDNSKNTLYLQMDSLRAEDTAVYYCAKEGDSSRWSYDLWGRGTLV |
| | p65 GR1 | | TVSSGGGGSGGGGSGGGGSQSALTQPASVSGSPGQSITISCTGTSSDVG |
| | Notch1NLS | | GYNYVSWYQQHPGKAPKVMIYDVTNRPSGVSNRFSGSKSGNTASLTISG |
| | | | LQAEDEADYYCSSYTIASTLVVFGGGTKLTVLTTTPAPRPPTPAPTIAS |
| | | | QPLSLRPEACFMYVAAAAFVLLFFVGCGVLLSKRKRKHRRKKSRPGERP |
| | | | FQCRICMRNFSRRHGLDRHTRTHTGEKPFQCRICMRNFSDHSSLKRHLR |
| | | | THTGSQKPFQCRICMRNFSVRHNLTRHLRTHTGEKPFQCRICMRNFSDH |
| | | | SNLSRHLKTHTGSQKPFQCRICMRNFSQRSSLVRHLRTHTGEKPFQCRI |
| | | | CMRNFSESGHLKRHLRTHLRDEFPTMVFPSGQISQASALAPAPPQVLPQ |
| | | | APAPAPAPAMVSALAQAPAPVPVLAPGPPQAVAPPAPKPTQAGEGTLSE |
| | | | ALLQLQFDDEDLGALLGNSTDPAVFTDLASVDNSEFQQLLNQGIPVAPH |
| | | | TTEPMLMEYPEAITRLVTGAQRPPDPAPAPLGAPGLPNGLLSGDEDFSS |
| | | | IADMDFSALLSQISSSGQGGGGSDQSTFDILQDLEFSSGSPGKETNESP |
| | | | WRSDLLIDENCLLSPLAGEDDSFLLEGNSNED |
| pDP1 | α-ALPPL2 | 162 | ATGGCGTTGCCTGTCACGGCACTGCTCCTTCCCCTTGCACTTCTTCTTC |
| 344 | HingeNotch | | ATGCAGCTCGCCCCGAGCAGAAATTGATCTCCGAAGAAGACCTTCAGGT |
| | ZF10 p65 | | CCAACTGCAGCAATCAGGTGGAGGACTTGTGAAACCAGGAGGGAGTCTG |
| | | | AGGCTTAGCTGCGCAGCGAGTGGGTTTACTTTCTCTAGTTACGCCATGC |
| | | | ACTGGGTGCGCCAAGCCCCCGGGAAAGGACTGGAGTGGGTCGCTGTTAT |
| | | | TTCATATGACGGGTCAAATAAATACTACGCCGATTCAGTAAAGGGTCGA |
| | | | TTTACTATATCTAGGGACAACAGTAAGAACACACTTTATCTGCAGATGG |
| | | | ATAGCCTGCGGGCGGAGGACACTGCAGTCTACTATTGCGCGAAGGAAGG |
| | | | AGACAGCTCACGCTGGAGCTACGACCTCTGGGGAAGGGGGACTCTCGTA |
| | | | ACGGTGTCATCCGGCGGTGGCGGCAGCGGTGGTGGTGGATCAGGGGGAG |
| | | | GGGGGTCTCAGTCAGCACTCACCCAACCCGCATCCGTGTCTGGTAGTCC |
| | | | GGGCCAGAGTATTACTATCTCCTGTACTGGGACATCCTCCGACGTTGGG |
| | | | GGCTATAACTACGTCAGCTGGTATCAACAGCACCCTGGTAAGGCACCCA |
| | | | AGGTAATGATCTATGATGTGACAAACAGACCCAGCGGTGTTTCTAACCG |
| | | | ATTTAGCGGTTCAAAATCAGGTAACACCGCAAGCCTTACCATATCTGGT |
| | | | CTCCAGGCTGAAGACGAAGCCGACTATTACTGCAGCAGCTATACCATAG |
| | | | CCTCTACACTGGTTGTCTTCGGAGGAGGCACTAAGCTGACTGTGCTAAC |
| | | | CACGACGCCAGCGCCGCGACCACCAACACCGGCGCCCACCATCGCGTCG |
| | | | CAGCCCCTGTCCCTGCGCCCAGAGGCGTGCTTTATGTACGTGGCAGCCG |
| | | | CGGCATTTGTACTTCTGTTTTTCGTGGGGTGCGGTGTGCTTCTCAGCAA |
| | | | GAGGAAACGGAAGCACcccaagaaaaagaggaaggtgTCTAGACCCGGA |
| | | | GAGCGCCCATTCCAGTGTCGGATTTGCATGCGGAACTTTTCGAGAAGAC |
| | | | ACGGCCTGGACAGACATACCCGTACTCATACAGGTGAAAAACCCTTTCA |
| | | | GTGTCGGATCTGTATGCGAAATTTCTCCGACCACAGCAGCCTGAAGAGA |
| | | | CATCTACGTACCCACACCGGCAGCCAGAAGCCATTTCAGTGTCGGATCT |
| | | | GTATGCGGAACTTCTCCGTGAGACACAACCTGACCAGACATCTACGTAC |
| | | | GCACACCGGAGAGAAGCCATTCCAATGCCGAATATGCATGCGCAACTTC |
| | | | AGTGACCACAGCAACCTGAGCAGACACCTAAAAACCCACACCGGTTCCC |
| | | | AGAAGCCATTTCAGTGTCGGATCTGTATGCGGAACTTCTCCCAGCGCAG |
| | | | CAGCCTGGTGAGACATCTACGTACGCACACCGGAGAGAAGCCATTCCAA |
| | | | TGCCGAATATGCATGCGCAACTTCAGTGAGAGCGGCCACCTGAAGAGAC |
| | | | ACCTGCGTACGCACCTGAGGGATGAGTTTCCCACCATGGTGTTTCCTTC |
| | | | TGGGCAGATCAGCCAGGCCTCGGCCTTGGCCCCGGCCCCTCCCCAAGTC |
| | | | CTGCCCCAGGCTCCAGCCCCTGCCCCTGCTCCAGCCATGGTATCAGCTC |
| | | | TGGCCCAGGCCCCAGCCCCTGTCCCAGTCCTAGCCCCAGGCCCTCCTCA |
| | | | GGCTGTGGCCCCACCTGCCCCCAAGCCCACCCAGGCTGGGGAAGGAACG |
| | | | CTGTCAGAGGCCCTGCTGCAGCTGCAGTTTGATGATGAAGACCTGGGGG |
| | | | CCTTGCTTGGCAACAGCACAGACCCAGCTGTGTTCACAGACCTGGCATC |
| | | | CGTCGACAACTCCGAGTTTCAGCAGCTGCTGAACCAGGGCATACCTGTG |
| | | | GCCCCCCACACAACTGAGCCCATGCTGATGGAGTACCCTGAGGCTATAA |
| | | | CTCGCCTAGTGACAGGGGCCCAGAGGCCCCCCGACCCAGCTCCTGCTCC |
| | | | ACTGGGGGCCCCGGGGCTCCCCAATGGCCTCCTTTCAGGAGATGAAGAC |
| | | | TTCTCCTCCATTGCGGACATGGACTTCTCAGCCCTGCTGAGTCAGATCA |
| | | | GCTCC |
| pDP1 | α-ALPPL2 | 163 | ATGGCGTTGCCTGTCACGGCACTGCTCCTTCCCCTTGCACTTCTTCTTC |
| 345 | HingeNotch | | ATGCAGCTCGCCCCGAGCAGAAATTGATCTCCGAAGAAGACCTTCAGGT |
| | ZF10 p65 | | CCAACTGCAGCAATCAGGTGGAGGACTTGTGAAACCAGGAGGGAGTCTG |
| | HSF1 | | AGGCTTAGCTGCGCAGCGAGTGGGTTTACTTTCTCTAGTTACGCCATGC |
| | | | ACTGGGTGCGCCAAGCCCCCGGGAAAGGACTGGAGTGGGTCGCTGTTAT |
| | | | TTCATATGACGGGTCAAATAAATACTACGCCGATTCAGTAAAGGGTCGA |
| | | | TTTACTATATCTAGGGACAACAGTAAGAACACACTTTATCTGCAGATGG |
| | | | ATAGCCTGCGGGCGGAGGACACTGCAGTCTACTATTGCGCGAAGGAAGG |
| | | | AGACAGCTCACGCTGGAGCTACGACCTCTGGGGAAGGGGGACTCTCGTA |
| | | | ACGGTGTCATCCGGCGGTGGCGGCAGCGGTGGTGGTGGATCAGGGGGAG |
| | | | GGGGGTCTCAGTCAGCACTCACCCAACCCGCATCCGTGTCTGGTAGTCC |
| | | | GGGCCAGAGTATTACTATCTCCTGTACTGGGACATCCTCCGACGTTGGG |
| | | | GGCTATAACTACGTCAGCTGGTATCAACAGCACCCTGGTAAGGCACCCA |
| | | | AGGTAATGATCTATGATGTGACAAACAGACCCAGCGGTGTTTCTAACCG |
| | | | ATTTAGCGGTTCAAAATCAGGTAACACCGCAAGCCTTACCATATCTGGT |
| | | | CTCCAGGCTGAAGACGAAGCCGACTATTACTGCAGCAGCTATACCATAG |
| | | | CCTCTACACTGGTTGTCTTCGGAGGAGGCACTAAGCTGACTGTGCTAAC |
| | | | CACGACGCCAGCGCCGCGACCACCAACACCGGCGCCCACCATCGCGTCG |
| | | | CAGCCCCTGTCCCTGCGCCCAGAGGCGTGCTTTATGTACGTGGCAGCCG |
| | | | CGGCATTTGTACTTCTGTTTTTCGTGGGGTGCGGTGTGCTTCTCAGCAA |
| | | | GAGGAAACGGAAGCACcccaagaaaaagaggaaggtgTCTAGACCCGGA |
| | | | GAGCGCCCATTCCAGTGTCGGATTTGCATGCGGAACTTTTCGAGAAGAC |
| | | | ACGGCCTGGACAGACATACCCGTACTCATACAGGTGAAAAACCCTTTCA |
| | | | GTGTCGGATCTGTATGCGAAATTTCTCCGACCACAGCAGCCTGAAGAGA |
| | | | CATCTACGTACCCACACCGGCAGCCAGAAGCCATTTCAGTGTCGGATCT |
| | | | GTATGCGGAACTTCTCCGTGAGACACAACCTGACCAGACATCTACGTAC |
| | | | GCACACCGGAGAGAAGCCATTCCAATGCCGAATATGCATGCGCAACTTC |
| | | | AGTGACCACAGCAACCTGAGCAGACACCTAAAAACCCACACCGGTTCCC |
| | | | AGAAGCCATTTCAGTGTCGGATCTGTATGCGGAACTTCTCCCAGCGCAG |
| | | | CAGCCTGGTGAGACATCTACGTACGCACACCGGAGAGAAGCCATTCCAA |
| | | | TGCCGAATATGCATGCGCAACTTCAGTGAGAGCGGCCACCTGAAGAGAC |
| | | | ACCTGCGTACGCACCTGAGGGATGAGTTTCCCACCATGGTGTTTCCTTC |
| | | | TGGGCAGATCAGCCAGGCCTCGGCCTTGGCCCCGGCCCCTCCCCAAGTC |
| | | | CTGCCCCAGGCTCCAGCCCCTGCCCCTGCTCCAGCCATGGTATCAGCTC |
| | | | TGGCCCAGGCCCCAGCCCCTGTCCCAGTCCTAGCCCCAGGCCCTCCTCA |
| | | | GGCTGTGGCCCCACCTGCCCCCAAGCCCACCCAGGCTGGGGAAGGAACG |
| | | | CTGTCAGAGGCCCTGCTGCAGCTGCAGTTTGATGATGAAGACCTGGGGG |
| | | | CCTTGCTTGGCAACAGCACAGACCCAGCTGTGTTCACAGACCTGGCATC |
| | | | CGTCGACAACTCCGAGTTTCAGCAGCTGCTGAACCAGGGCATACCTGTG |
| | | | GCCCCCCACACAACTGAGCCCATGCTGATGGAGTACCCTGAGGCTATAA |
| | | | CTCGCCTAGTGACAGGGGCCCAGAGGCCCCCCGACCCAGCTCCTGCTCC |
| | | | ACTGGGGGCCCCGGGGCTCCCCAATGGCCTCCTTTCAGGAGATGAAGAC |
| | | | TTCTCCTCCATTGCGGACATGGACTTCTCAGCCCTGCTGAGTCAGATCA |
| | | | GCTCCAGTGGGCAGGGAGGAGGTGGAAGCGGCTTCAGCGTGGACACCAG |
| | | | TGCCCTGCTGGACCTGTTCAGCCCCTCGGTGACCGTGCCCGACATGAGC |
| | | | CTGCCTGACCTTGACAGCAGCCTGGCCAGTATCCAAGAGCTCCTGTCTC |
| | | | CCCAGGAGCCCCCCAGGCCTCCCGAGGCAGAGAACAGCAGCCCGGATTC |
| | | | AGGGAAGCAGCTGGTGCACTACACAGCGCAGCCGCTGTTCCTGCTGGAC |
| | | | CCCGGCTCCGTGGACACCGGGAGCAACGACCTGCCGGTGCTGTTTGAGC |
| | | | TGGGAGAGGGCTCCTACTTCTCCGAAGGGGACGGCTTCGCCGAGGACCC |
| | | | CACCATCTCCCTGCTGACAGGCTCGGAGCCTCCCAAAGCCAAGGACCCC |
| | | | ACTGTCTCC |
| pDP1 | α-ALPPL2 | 164 | ATGGCGTTGCCTGTCACGGCACTGCTCCTTCCCCTTGCACTTCTTCTTC |
| 346 | HingeNotch | | ATGCAGCTCGCCCCGAGCAGAAATTGATCTCCGAAGAAGACCTTCAGGT |
| | ZF10 p65 GR1 | | CCAACTGCAGCAATCAGGTGGAGGACTTGTGAAACCAGGAGGGAGTCTG |
| | | | AGGCTTAGCTGCGCAGCGAGTGGGTTTACTTTCTCTAGTTACGCCATGC |
| | | | ACTGGGTGCGCCAAGCCCCCGGGAAAGGACTGGAGTGGGTCGCTGTTAT |
| | | | TTCATATGACGGGTCAAATAAATACTACGCCGATTCAGTAAAGGGTCGA |
| | | | TTTACTATATCTAGGGACAACAGTAAGAACACACTTTATCTGCAGATGG |
| | | | ATAGCCTGCGGGCGGAGGACACTGCAGTCTACTATTGCGCGAAGGAAGG |
| | | | AGACAGCTCACGCTGGAGCTACGACCTCTGGGGAAGGGGGACTCTCGTA |
| | | | ACGGTGTCATCCGGCGGTGGCGGCAGCGGTGGTGGTGGATCAGGGGGAG |
| | | | GGGGGTCTCAGTCAGCACTCACCCAACCCGCATCCGTGTCTGGTAGTCC |
| | | | GGGCCAGAGTATTACTATCTCCTGTACTGGGACATCCTCCGACGTTGGG |
| | | | GGCTATAACTACGTCAGCTGGTATCAACAGCACCCTGGTAAGGCACCCA |
| | | | AGGTAATGATCTATGATGTGACAAACAGACCCAGCGGTGTTTCTAACCG |
| | | | ATTTAGCGGTTCAAAATCAGGTAACACCGCAAGCCTTACCATATCTGGT |
| | | | CTCCAGGCTGAAGACGAAGCCGACTATTACTGCAGCAGCTATACCATAG |
| | | | CCTCTACACTGGTTGTCTTCGGAGGAGGCACTAAGCTGACTGTGCTAAC |
| | | | CACGACGCCAGCGCCGCGACCACCAACACCGGCGCCCACCATCGCGTCG |
| | | | CAGCCCCTGTCCCTGCGCCCAGAGGCGTGCTTTATGTACGTGGCAGCCG |
| | | | CGGCATTTGTACTTCTGTTTTTCGTGGGGTGCGGTGTGCTTCTCAGCAA |
| | | | GAGGAAACGGAAGCACcccaagaaaaagaggaaggtgTCTAGACCCGGA |
| | | | GAGCGCCCATTCCAGTGTCGGATTTGCATGCGGAACTTTTCGAGAAGAC |
| | | | ACGGCCTGGACAGACATACCCGTACTCATACAGGTGAAAAACCCTTTCA |
| | | | GTGTCGGATCTGTATGCGAAATTTCTCCGACCACAGCAGCCTGAAGAGA |
| | | | CATCTACGTACCCACACCGGCAGCCAGAAGCCATTTCAGTGTCGGATCT |
| | | | GTATGCGGAACTTCTCCGTGAGACACAACCTGACCAGACATCTACGTAC |
| | | | GCACACCGGAGAGAAGCCATTCCAATGCCGAATATGCATGCGCAACTTC |
| | | | AGTGACCACAGCAACCTGAGCAGACACCTAAAAACCCACACCGGTTCCC |
| | | | AGAAGCCATTTCAGTGTCGGATCTGTATGCGGAACTTCTCCCAGCGCAG |
| | | | CAGCCTGGTGAGACATCTACGTACGCACACCGGAGAGAAGCCATTCCAA |
| | | | TGCCGAATATGCATGCGCAACTTCAGTGAGAGCGGCCACCTGAAGAGAC |
| | | | ACCTGCGTACGCACCTGAGGGATGAGTTTCCCACCATGGTGTTTCCTTC |
| | | | TGGGCAGATCAGCCAGGCCTCGGCCTTGGCCCCGGCCCCTCCCCAAGTC |
| | | | CTGCCCCAGGCTCCAGCCCCTGCCCCTGCTCCAGCCATGGTATCAGCTC |
| | | | TGGCCCAGGCCCCAGCCCCTGTCCCAGTCCTAGCCCCAGGCCCTCCTCA |
| | | | GGCTGTGGCCCCACCTGCCCCCAAGCCCACCCAGGCTGGGGAAGGAACG |
| | | | CTGTCAGAGGCCCTGCTGCAGCTGCAGTTTGATGATGAAGACCTGGGGG |
| | | | CCTTGCTTGGCAACAGCACAGACCCAGCTGTGTTCACAGACCTGGCATC |
| | | | CGTCGACAACTCCGAGTTTCAGCAGCTGCTGAACCAGGGCATACCTGTG |
| | | | GCCCCCCACACAACTGAGCCCATGCTGATGGAGTACCCTGAGGCTATAA |
| | | | CTCGCCTAGTGACAGGGGCCCAGAGGCCCCCCGACCCAGCTCCTGCTCC |
| | | | ACTGGGGGCCCCGGGGCTCCCCAATGGCCTCCTTTCAGGAGATGAAGAC |
| | | | TTCTCCTCCATTGCGGACATGGACTTCTCAGCCCTGCTGAGTCAGATCA |
| | | | GCTCCAGTGGGCAGGGAGGAGGTGGAAGCGACCAAAGCACCTTTGACAT |
| | | | TTTGCAGGATTTGGAGTTTTCTTCTGGGTCCCCAGGTAAAGAGACGAAT |
| | | | GAGAGTCCTTGGAGATCAGACCTGTTGATAGATGAAAACTGTTTGCTTT |
| | | | CTCCTCTGGCGGGAGAAGACGATTCATTCCTTTTGGAAGGAAACTCGAA |
| | | | TGAGGAC |
| pDP1 | α-ALPPL2 | 165 | ATGGCGTTGCCTGTCACGGCACTGCTCCTTCCCCTTGCACTTCTTCTTC |
| 347 | HingeNotch | | ATGCAGCTCGCCCCGAGCAGAAATTGATCTCCGAAGAAGACCTTCAGGT |
| | ZF10 p65 | | CCAACTGCAGCAATCAGGTGGAGGACTTGTGAAACCAGGAGGGAGTCTG |
| | NotchlNLS | | AGGCTTAGCTGCGCAGCGAGTGGGTTTACTTTCTCTAGTTACGCCATGC |
| | | | ACTGGGTGCGCCAAGCCCCCGGGAAAGGACTGGAGTGGGTCGCTGTTAT |
| | | | TTCATATGACGGGTCAAATAAATACTACGCCGATTCAGTAAAGGGTCGA |
| | | | TTTACTATATCTAGGGACAACAGTAAGAACACACTTTATCTGCAGATGG |
| | | | ATAGCCTGCGGGCGGAGGACACTGCAGTCTACTATTGCGCGAAGGAAGG |
| | | | AGACAGCTCACGCTGGAGCTACGACCTCTGGGGAAGGGGGACTCTCGTA |
| | | | ACGGTGTCATCCGGCGGTGGCGGCAGCGGTGGTGGTGGATCAGGGGGAG |
| | | | GGGGGTCTCAGTCAGCACTCACCCAACCCGCATCCGTGTCTGGTAGTCC |
| | | | GGGCCAGAGTATTACTATCTCCTGTACTGGGACATCCTCCGACGTTGGG |
| | | | GGCTATAACTACGTCAGCTGGTATCAACAGCACCCTGGTAAGGCACCCA |
| | | | AGGTAATGATCTATGATGTGACAAACAGACCCAGCGGTGTTTCTAACCG |
| | | | ATTTAGCGGTTCAAAATCAGGTAACACCGCAAGCCTTACCATATCTGGT |
| | | | CTCCAGGCTGAAGACGAAGCCGACTATTACTGCAGCAGCTATACCATAG |
| | | | CCTCTACACTGGTTGTCTTCGGAGGAGGCACTAAGCTGACTGTGCTAAC |
| | | | CACGACGCCAGCGCCGCGACCACCAACACCGGCGCCCACCATCGCGTCG |
| | | | CAGCCCCTGTCCCTGCGCCCAGAGGCGTGCTTTATGTACGTGGCAGCCG |
| | | | CGGCATTTGTACTTCTGTTTTTCGTGGGGTGCGGTGTGCTTCTCAGCAA |
| | | | GAGGAAACGGAAGCACcggaggaagaagTCTAGACCCGGAGAGCGCCCA |
| | | | TTCCAGTGTCGGATTTGCATGCGGAACTTTTCGAGAAGACACGGCCTGG |
| | | | ACAGACATACCCGTACTCATACAGGTGAAAAACCCTTTCAGTGTCGGAT |
| | | | CTGTATGCGAAATTTCTCCGACCACAGCAGCCTGAAGAGACATCTACGT |
| | | | ACCCACACCGGCAGCCAGAAGCCATTTCAGTGTCGGATCTGTATGCGGA |
| | | | ACTTCTCCGTGAGACACAACCTGACCAGACATCTACGTACGCACACCGG |
| | | | AGAGAAGCCATTCCAATGCCGAATATGCATGCGCAACTTCAGTGACCAC |
| | | | AGCAACCTGAGCAGACACCTAAAAACCCACACCGGTTCCCAGAAGCCAT |
| | | | TTCAGTGTCGGATCTGTATGCGGAACTTCTCCCAGCGCAGCAGCCTGGT |
| | | | GAGACATCTACGTACGCACACCGGAGAGAAGCCATTCCAATGCCGAATA |
| | | | TGCATGCGCAACTTCAGTGAGAGCGGCCACCTGAAGAGACACCTGCGTA |
| | | | CGCACCTGAGGGATGAGTTTCCCACCATGGTGTTTCCTTCTGGGCAGAT |
| | | | CAGCCAGGCCTCGGCCTTGGCCCCGGCCCCTCCCCAAGTCCTGCCCCAG |
| | | | GCTCCAGCCCCTGCCCCTGCTCCAGCCATGGTATCAGCTCTGGCCCAGG |
| | | | CCCCAGCCCCTGTCCCAGTCCTAGCCCCAGGCCCTCCTCAGGCTGTGGC |
| | | | CCCACCTGCCCCCAAGCCCACCCAGGCTGGGGAAGGAACGCTGTCAGAG |
| | | | GCCCTGCTGCAGCTGCAGTTTGATGATGAAGACCTGGGGGCCTTGCTTG |
| | | | GCAACAGCACAGACCCAGCTGTGTTCACAGACCTGGCATCCGTCGACAA |
| | | | CTCCGAGTTTCAGCAGCTGCTGAACCAGGGCATACCTGTGGCCCCCCAC |
| | | | ACAACTGAGCCCATGCTGATGGAGTACCCTGAGGCTATAACTCGCCTAG |
| | | | TGACAGGGGCCCAGAGGCCCCCCGACCCAGCTCCTGCTCCACTGGGGGC |
| | | | CCCGGGGCTCCCCAATGGCCTCCTTTCAGGAGATGAAGACTTCTCCTCC |
| | | | ATTGCGGACATGGACTTCTCAGCCCTGCTGAGTCAGATCAGCTCC |
| pDP1 | α-ALPPL2 | 166 | ATGGCGTTGCCTGTCACGGCACTGCTCCTTCCCCTTGCACTTCTTCTTC |
| 348 | HingeNotch | | ATGCAGCTCGCCCCGAGCAGAAATTGATCTCCGAAGAAGACCTTCAGGT |
| | ZF10 p65 | | CCAACTGCAGCAATCAGGTGGAGGACTTGTGAAACCAGGAGGGAGTCTG |
| | HSF1 | | AGGCTTAGCTGCGCAGCGAGTGGGTTTACTTTCTCTAGTTACGCCATGC |
| | NotchlNLS | | ACTGGGTGCGCCAAGCCCCCGGGAAAGGACTGGAGTGGGTCGCTGTTAT |
| | | | TTCATATGACGGGTCAAATAAATACTACGCCGATTCAGTAAAGGGTCGA |
| | | | TTTACTATATCTAGGGACAACAGTAAGAACACACTTTATCTGCAGATGG |
| | | | ATAGCCTGCGGGCGGAGGACACTGCAGTCTACTATTGCGCGAAGGAAGG |
| | | | AGACAGCTCACGCTGGAGCTACGACCTCTGGGGAAGGGGGACTCTCGTA |
| | | | ACGGTGTCATCCGGCGGTGGCGGCAGCGGTGGTGGTGGATCAGGGGGAG |
| | | | GGGGGTCTCAGTCAGCACTCACCCAACCCGCATCCGTGTCTGGTAGTCC |
| | | | GGGCCAGAGTATTACTATCTCCTGTACTGGGACATCCTCCGACGTTGGG |
| | | | GGCTATAACTACGTCAGCTGGTATCAACAGCACCCTGGTAAGGCACCCA |
| | | | AGGTAATGATCTATGATGTGACAAACAGACCCAGCGGTGTTTCTAACCG |
| | | | ATTTAGCGGTTCAAAATCAGGTAACACCGCAAGCCTTACCATATCTGGT |
| | | | CTCCAGGCTGAAGACGAAGCCGACTATTACTGCAGCAGCTATACCATAG |
| | | | CCTCTACACTGGTTGTCTTCGGAGGAGGCACTAAGCTGACTGTGCTAAC |
| | | | CACGACGCCAGCGCCGCGACCACCAACACCGGCGCCCACCATCGCGTCG |
| | | | CAGCCCCTGTCCCTGCGCCCAGAGGCGTGCTTTATGTACGTGGCAGCCG |
| | | | CGGCATTTGTACTTCTGTTTTTCGTGGGGTGCGGTGTGCTTCTCAGCAA |
| | | | GAGGAAACGGAAGCACcggaggaagaagTCTAGACCCGGAGAGCGCCCA |
| | | | TTCCAGTGTCGGATTTGCATGCGGAACTTTTCGAGAAGACACGGCCTGG |
| | | | ACAGACATACCCGTACTCATACAGGTGAAAAACCCTTTCAGTGTCGGAT |
| | | | CTGTATGCGAAATTTCTCCGACCACAGCAGCCTGAAGAGACATCTACGT |
| | | | ACCCACACCGGCAGCCAGAAGCCATTTCAGTGTCGGATCTGTATGCGGA |
| | | | ACTTCTCCGTGAGACACAACCTGACCAGACATCTACGTACGCACACCGG |
| | | | AGAGAAGCCATTCCAATGCCGAATATGCATGCGCAACTTCAGTGACCAC |
| | | | AGCAACCTGAGCAGACACCTAAAAACCCACACCGGTTCCCAGAAGCCAT |
| | | | TTCAGTGTCGGATCTGTATGCGGAACTTCTCCCAGCGCAGCAGCCTGGT |
| | | | GAGACATCTACGTACGCACACCGGAGAGAAGCCATTCCAATGCCGAATA |
| | | | TGCATGCGCAACTTCAGTGAGAGCGGCCACCTGAAGAGACACCTGCGTA |
| | | | CGCACCTGAGGGATGAGTTTCCCACCATGGTGTTTCCTTCTGGGCAGAT |
| | | | CAGCCAGGCCTCGGCCTTGGCCCCGGCCCCTCCCCAAGTCCTGCCCCAG |
| | | | GCTCCAGCCCCTGCCCCTGCTCCAGCCATGGTATCAGCTCTGGCCCAGG |
| | | | CCCCAGCCCCTGTCCCAGTCCTAGCCCCAGGCCCTCCTCAGGCTGTGGC |
| | | | CCCACCTGCCCCCAAGCCCACCCAGGCTGGGGAAGGAACGCTGTCAGAG |
| | | | GCCCTGCTGCAGCTGCAGTTTGATGATGAAGACCTGGGGGCCTTGCTTG |
| | | | GCAACAGCACAGACCCAGCTGTGTTCACAGACCTGGCATCCGTCGACAA |
| | | | CTCCGAGTTTCAGCAGCTGCTGAACCAGGGCATACCTGTGGCCCCCCAC |
| | | | ACAACTGAGCCCATGCTGATGGAGTACCCTGAGGCTATAACTCGCCTAG |
| | | | TGACAGGGGCCCAGAGGCCCCCCGACCCAGCTCCTGCTCCACTGGGGGC |
| | | | CCCGGGGCTCCCCAATGGCCTCCTTTCAGGAGATGAAGACTTCTCCTCC |
| | | | ATTGCGGACATGGACTTCTCAGCCCTGCTGAGTCAGATCAGCTCCAGTG |
| | | | GGCAGGGAGGAGGTGGAAGCGGCTTCAGCGTGGACACCAGTGCCCTGCT |
| | | | GGACCTGTTCAGCCCCTCGGTGACCGTGCCCGACATGAGCCTGCCTGAC |
| | | | CTTGACAGCAGCCTGGCCAGTATCCAAGAGCTCCTGTCTCCCCAGGAGC |
| | | | CCCCCAGGCCTCCCGAGGCAGAGAACAGCAGCCCGGATTCAGGGAAGCA |
| | | | GCTGGTGCACTACACAGCGCAGCCGCTGTTCCTGCTGGACCCCGGCTCC |
| | | | GTGGACACCGGGAGCAACGACCTGCCGGTGCTGTTTGAGCTGGGAGAGG |
| | | | GCTCCTACTTCTCCGAAGGGGACGGCTTCGCCGAGGACCCCACCATCTC |
| | | | CCTGCTGACAGGCTCGGAGCCTCCCAAAGCCAAGGACCCCACTGTCTCC |
| pDP1 | α-ALPPL2 | 167 | ATGGCGTTGCCTGTCACGGCACTGCTCCTTCCCCTTGCACTTCTTCTTC |
| 349 | HingeNotch | | ATGCAGCTCGCCCCGAGCAGAAATTGATCTCCGAAGAAGACCTTCAGGT |
| | ZF10 p65 GR1 | | CCAACTGCAGCAATCAGGTGGAGGACTTGTGAAACCAGGAGGGAGTCTG |
| | Notch1NLS | | AGGCTTAGCTGCGCAGCGAGTGGGTTTACTTTCTCTAGTTACGCCATGC |
| | | | ACTGGGTGCGCCAAGCCCCCGGGAAAGGACTGGAGTGGGTCGCTGTTAT |
| | | | TTCATATGACGGGTCAAATAAATACTACGCCGATTCAGTAAAGGGTCGA |
| | | | TTTACTATATCTAGGGACAACAGTAAGAACACACTTTATCTGCAGATGG |
| | | | ATAGCCTGCGGGCGGAGGACACTGCAGTCTACTATTGCGCGAAGGAAGG |
| | | | AGACAGCTCACGCTGGAGCTACGACCTCTGGGGAAGGGGGACTCTCGTA |
| | | | ACGGTGTCATCCGGCGGTGGCGGCAGCGGTGGTGGTGGATCAGGGGGAG |
| | | | GGGGGTCTCAGTCAGCACTCACCCAACCCGCATCCGTGTCTGGTAGTCC |
| | | | GGGCCAGAGTATTACTATCTCCTGTACTGGGACATCCTCCGACGTTGGG |
| | | | GGCTATAACTACGTCAGCTGGTATCAACAGCACCCTGGTAAGGCACCCA |
| | | | AGGTAATGATCTATGATGTGACAAACAGACCCAGCGGTGTTTCTAACCG |
| | | | ATTTAGCGGTTCAAAATCAGGTAACACCGCAAGCCTTACCATATCTGGT |
| | | | CTCCAGGCTGAAGACGAAGCCGACTATTACTGCAGCAGCTATACCATAG |
| | | | CCTCTACACTGGTTGTCTTCGGAGGAGGCACTAAGCTGACTGTGCTAAC |
| | | | CACGACGCCAGCGCCGCGACCACCAACACCGGCGCCCACCATCGCGTCG |
| | | | CAGCCCCTGTCCCTGCGCCCAGAGGCGTGCTTTATGTACGTGGCAGCCG |
| | | | CGGCATTTGTACTTCTGTTTTTCGTGGGGTGCGGTGTGCTTCTCAGCAA |
| | | | GAGGAAACGGAAGCACcggaggaagaagTCTAGACCCGGAGAGCGCCCA |
| | | | TTCCAGTGTCGGATTTGCATGCGGAACTTTTCGAGAAGACACGGCCTGG |
| | | | ACAGACATACCCGTACTCATACAGGTGAAAAACCCTTTCAGTGTCGGAT |
| | | | CTGTATGCGAAATTTCTCCGACCACAGCAGCCTGAAGAGACATCTACGT |
| | | | ACCCACACCGGCAGCCAGAAGCCATTTCAGTGTCGGATCTGTATGCGGA |
| | | | ACTTCTCCGTGAGACACAACCTGACCAGACATCTACGTACGCACACCGG |
| | | | AGAGAAGCCATTCCAATGCCGAATATGCATGCGCAACTTCAGTGACCAC |
| | | | AGCAACCTGAGCAGACACCTAAAAACCCACACCGGTTCCCAGAAGCCAT |
| | | | TTCAGTGTCGGATCTGTATGCGGAACTTCTCCCAGCGCAGCAGCCTGGT |
| | | | GAGACATCTACGTACGCACACCGGAGAGAAGCCATTCCAATGCCGAATA |
| | | | TGCATGCGCAACTTCAGTGAGAGCGGCCACCTGAAGAGACACCTGCGTA |
| | | | CGCACCTGAGGGATGAGTTTCCCACCATGGTGTTTCCTTCTGGGCAGAT |
| | | | CAGCCAGGCCTCGGCCTTGGCCCCGGCCCCTCCCCAAGTCCTGCCCCAG |
| | | | GCTCCAGCCCCTGCCCCTGCTCCAGCCATGGTATCAGCTCTGGCCCAGG |
| | | | CCCCAGCCCCTGTCCCAGTCCTAGCCCCAGGCCCTCCTCAGGCTGTGGC |
| | | | CCCACCTGCCCCCAAGCCCACCCAGGCTGGGGAAGGAACGCTGTCAGAG |
| | | | GCCCTGCTGCAGCTGCAGTTTGATGATGAAGACCTGGGGGCCTTGCTTG |
| | | | GCAACAGCACAGACCCAGCTGTGTTCACAGACCTGGCATCCGTCGACAA |
| | | | CTCCGAGTTTCAGCAGCTGCTGAACCAGGGCATACCTGTGGCCCCCCAC |
| | | | ACAACTGAGCCCATGCTGATGGAGTACCCTGAGGCTATAACTCGCCTAG |
| | | | TGACAGGGGCCCAGAGGCCCCCCGACCCAGCTCCTGCTCCACTGGGGGC |
| | | | CCCGGGGCTCCCCAATGGCCTCCTTTCAGGAGATGAAGACTTCTCCTCC |
| | | | ATTGCGGACATGGACTTCTCAGCCCTGCTGAGTCAGATCAGCTCCAGTG |
| | | | GGCAGGGAGGAGGTGGAAGCGACCAAAGCACCTTTGACATTTTGCAGGA |
| | | | TTTGGAGTTTTCTTCTGGGTCCCCAGGTAAAGAGACGAATGAGAGTCCT |
| | | | TGGAGATCAGACCTGTTGATAGATGAAAACTGTTTGCTTTCTCCTCTGG |
| | | | CGGGAGAAGACGATTCATTCCTTTTGGAAGGAAACTCGAATGAGGAC |

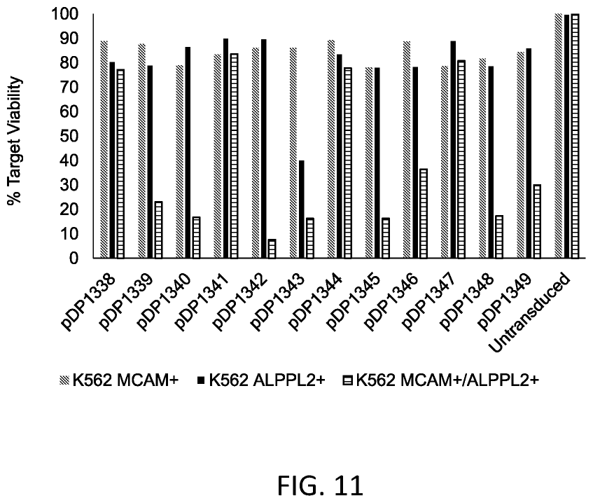

In these experiments, primary human CD8+ T-cells bearing α-ALPPL2 HingeNotch receptors containing (i) ZF6 or ZF10 DNA binding domains, (ii) SV40 or Notch1 NLS sequences, and (iii) p65, p65-HSF1, or p65-GR TADs were co-incubated with K562 target cells for 72 hours. All T-cells were co-transduced with a cognate zinc finger-responsive promoter driving expression of an α-MCAM CAR. K562s were either unmodified (MCAM+), transduced with ALPPL2 (MCAM+/ALPPL2+), or transduced with ALPPL2 and rendered deficient in MCAM expression via CRISPR knockout (ALPPL2+). As shown in FIG. 11, the ZF6 and ZF10-based receptor-driven CARs were able to specifically kill MCAM+/ALPPL2+K562 cells when incorporating the augmented p65-GR1 or p65-HSF1 TADs. Notably, the ALPPL2 bar (the dark black bar in the middle of the three) for construct pDP1343 is lower than expected, near the level of MCAM/ALPPL2 (the hatched bar). It is noted that this is likely due to a technical error, e.g., pipetting error, and thus could be considered an outlier.

### Example 14

### General Experimental Procedures

### Design of Receptor and Response Element Construct

Receptors were built by fusing the CD19 scFv to the corresponding SynTF obtained from the Khalil laboratory. All receptors contain an N-terminal CD8α signal peptide (MALPVTALLLPLALLLHAARP; SEQ ID NO: 72) for membrane targeting and a myc-tag (EQKLISEEDL; SEQ ID NO: 73) to facilitate determination of surface expression with α-myc A647 (Cell-Signaling #2233). The receptors were then cloned into a modified pHR′ SIN:CSW vector containing a phosphoglycerate kinase (PGK) promoter.

The pHR′ SIN:CSW vector was also modified to make the response element plasmids. Eight copies of the ZFA target sequence 3 ZF3 (aGACGTCGAAGTAGCCGTAg; SEQ ID NO: 63), ZFA target sequence ZF6 (gGACGACGCGGTCTAAGAAg; SEQ ID NO: 66), or ZFA target sequence ZF10 (cGGCGTAGCCGATGTCGCGc; SEQ ID NO: 70) DNA binding domain target sequence were cloned 5′ to a minimal ybTATA promoter driving a tagBFP gene. Also included in the response element plasmids was a PGK promoter that constitutively drives mCitrine expression to suitably identify transduced T cells. For all inducible BFP vectors, BFP was cloned via a BamHI site in the multiple cloning site 3′ to the ybTATA promoter. For evaluation of target-specific killing performance, the reporter construct was modified as follows. The constitutively-expressed mCitrine was replaced with BFP, while the inducible BFP was replaced with an anti-MCAM CAR (M1). For ALPPL2 targeting, the synZTE-containing HingeNotch receptor constructs were modified to replace the scFv with the appropriate ALPPL2-specific scFv variant M25FYIA. All constructs were cloned via Gibson HiFi assembly (NEB #E2621L).

### Isolation and Culture of Primary Human T Cells

Primary CD4+ or CD8+ T cells were isolated from anonymous donor blood after apheresis by negative selection (STEMCELL Technologies #15062 & 15063). Blood was obtained from Blood Centers of the Pacific (San Francisco, Calif.) as approved by the University Institutional Review Board. T cells were cryopreserved in RPMI-1640 (UCSF cell culture core) with 20% human AB serum (Valley Biomedical Inc., #HP1022) and 10% DMSO. After thawing, T cells were cultured in human T cell medium consisting of X-VIVO 15 (Lonza #04-418Q), 5% Human AB serum and 10 mM neutralized N-acetyl L-Cysteine (Sigma-Aldrich #A9165) supplemented with 30 units/mL IL-2 (NCI BRB Preclinical Repository) for all experiments.

### Lentiviral Transduction of Human T Cells

Pantropic VSV-G pseudotyped lentivirus was produced via transfection of Lenti-X 293T cells (Clontech #11131D) with a transgene expression vector constructed as described above and the viral packaging plasmids pCMVdR8.91 and pMD2.G using Mirus TransIT-Lenti (Mirus #MIR 6606). Primary T cells were thawed the same day, and after 24 hours in culture, were stimulated with Human T-Activator CD3/CD28 Dynabeads (Life Technologies #11131D) at a cell:bead ratio of 1:3. At 48 hours, viral supernatant was harvested and the primary T cells were exposed to the virus for 24 hours. At day 5 post T cell stimulation, the Dynabeads were removed, and the T cells expanded until day 10 when they were rested and could be used in assays. T cells were sorted for assays with a Beckton Dickinson (BD) FACs ARIA II.

### Generation of Reporter Jurkat T Cells for TMD/STS Screen

E6-1 Jurkat T cells (ATCC #TIB-152) were lentivirally transduced with a reporter plasmid with inducible BFP and constitutive mCitrine, as described previously (Roybal et al. 2016, supra). Reporter-positive Jurkat cells were sorted for mCitrine expression using a Beckton Dickinson (BD) FACs ARIA II and expanded. Lentivirus was produced with the receptor transgene expression vector as described previously. Reporter-positive Jurkat cells were transduced with receptor and expanded for experimentation in 96 well plates.

### Cancer Cell Lines

The target cell lines for all experiments were K562 myelogenous leukemia cells (ATCC #CCL-243). K562^{CD19+} were generated via lentiviral transduction to stably express human CD19 at equivalent levels as Daudi tumors. CD19 levels were determined by staining the cells with anti-CD19 APC (Biolegend #302212).

For ALPPL2 targeting, the K562 cells were transduced with a lentivirus expressing the ALPPL2 transgene (NP_112603.2) in a modified pHR′ SIN:CSW vector containing a silencing-prone spleen focus forming virus (SFFV) promoter to generate K562 MCAM+/ALPPL2+. To generate the K562 ALPPL2+ cell line, K562 MCAM+/ALPPL2+ were subjected to CRISPR knockout at the MCAM locus using the gRNA sequence AGGCGCAGCTCCCGGGCTGG (SEQ ID NO: 190), which was cloned into the pL-CRISPR.EFS.GFP vector, a gift from Benjamin Ebert (Addgene plasmid #57818). These constructs were cloned via In-Fusion cloning (Takara Bio).

### In Vitro Stimulation of Primary T Cells

For all in vitro T cell stimulations, 1×10⁵ T cells were co-cultured with sender cells at a 1:1 ratio in round bottom 96-well tissue culture plates. The cultures were analyzed at 48 hours for reporter activation with a BD Fortessa X-50. All flow cytometry analysis was performed in FlowJo software (TreeStar).

### In Vitro Stimulation of Jurkat T Cells

For all in vitro Jurkat T cell stimulations, 1×10⁵ Jurkat T cells were co-cultured with sender cells at a 1:1 ratio in round bottom 96-well tissue culture plates. The cultures were analyzed at 48 hours for reporter activation with a BD Fortessa X-50. All flow cytometry analysis was performed in FlowJo software (TreeStar).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

Dudani J. S., Warren A. D., and Bhatia S. N., Harnessing Protease Activity to Improve Cancer Care. Annu. Rev. Cancer Biol. 2018. 2:353-76.
David L. Porter, M. D., Bruce L. Levine, Ph.D., Michael Kalos, Ph.D., Adam Bagg, M. D., and Carl H. June, M.D. Chimeric Antigen Receptor-Modified T Cells in Chronic Lymphoid Leukemia. N. Engl J Med. 2011 Aug. 25; 365(8):725-33.
Gordon W R et al., The molecular logic of Notch signaling—a structural and biochemical perspective. J. Cell Sci. (2008) 121:3109-19.
Gordon W R et al., Mechanical Allostery: Evidence for a Force Requirement in the Proteolytic Activation of Notch. Dev Cell (2015) 33:729-36.
Morsut L, Roybal K T, Xiong X, Gordley R M, Coyle S M, Thomson M, and Lim W A. Engineering Customized Cell Sensing and Response Behaviors Using Synthetic Notch Receptors. Cell. 2016 Feb. 11; 164(4):780-91.
Naso M F, Tomkowicz B, Perry W L 3rd, Strohl W R. Adeno-Associated Virus (AAV) as a Vector for Gene Therapy. BioDrugs. 2017; 31(4):317-34.
Nasri M, Karimi A, Allahbakhshian Farsani M. Production, purification and titration of a lentivirus-based vector for gene delivery purposes. Cytotechnology. 2014; 66(6):1031-38.
Roybal K T, Jasper Z. Williams, Leonardo Morsut, Levi J. Rupp, Isabel Kolinko, Joseph H. Choe, Whitney J. Walker, Krista A. McNally, and Wendell A. Lim. Engineering T cells with Customized Therapeutic Response Programs Using Synthetic Notch Receptors. Cell. 2016 Oct. 6; 167(2):419-32.
Samulski and Muzyczka (2014). AAV-Mediated Gene Therapy for Research and Therapeutic Purposes. Annu. Rev. Virol. 1:427.
Sakuma, et al. (2012). Lentiviral vectors: basic to translational. Biochem. J. 443:603.
Watson D. J., Wolfe J. H. Viral vectors for gene therapy: methods and protocols. Totowa, N.J., USA: Humana Press; 2003. pp. 383-404.
Vidarsson G. et al., IgG subclasses and allotypes: from structure to effector functions. Frontiers Immunol. (2014) Oct. 20; 5:520.
F. An, D. C. Drummond, S. Wilson, D. B. Kirpotin, S. L. Nishimura, V. C. Broaddus, B. Liu, Targeted drug delivery to mesothelioma cells using functionally selected internalizing human single-chain antibodies, Mol. Cancer Ther. 7, 569-578 (2008).
Y. Su, X. Zhang, S. Bidlingmaier, C. R. Behrens, N.-K. Lee, B. Liu, ALPPL2 Is a Highly Specific and Targetable Tumor Cell Surface Antigen, Cancer Res. 80, 4552-4564 (2020).

What is claimed is:

1. A chimeric polypeptide comprising, from N-terminus to C-terminus:
a) an extracellular ligand-binding domain having a binding affinity for a selected ligand;
b) a linking polypeptide having:
(i) at least about 80%, 85%, 90%, 95%, 96%, 97%, 98%, 99%, or 100% sequence identity to a Notch juxtamembrane domain (JMD) wherein a LIN-12-Notch repeat (LNR) and/or a heterodimerization domain (HD) of a Notch receptor has been deleted;
(ii) at least about 80%, 85%, 90%, 95%, 96%, 97%, 98%, 99%, or 100% sequence identity to a polypeptide hinge domain; or
(iii) a sequence of about 2 to about 40 amino acid residues;
c) a transmembrane domain having at least about 80%, 85%, 90%, 95%, 96%, 97%, 98%, 99%, or 100% sequence identity to the transmembrane domain of a Type 1 transmembrane receptor and comprising one or more ligand-inducible proteolytic cleavage sites; and
d) an intracellular domain comprising a zinc finger-containing transcriptional effector (ZTE), wherein binding of the selected ligand to the extracellular binding domain induces cleavage at a ligand-inducible proteolytic cleavage site within the transmembrane domain.

2. The chimeric polypeptide of claim 1, further comprising a stop-transfer-sequence (STS) in between the transmembrane domain and the intracellular domain.

3. The chimeric polypeptide of any one of claims 1 to 2, wherein the linking polypeptide comprises an amino acid sequence having at least about 80%, 85%, 90%, 95%, 96%, 97%, 98%, 99%, or 100% sequence identity to a Notch JMD according to any one of SEQ ID NOS: 11-19.

4. The chimeric polypeptide of any one of claims 1 to 2, wherein the linking polypeptide has a length ranging from 1 to 40 amino acid residues.

5. The chimeric polypeptide of claim 4, wherein the linking polypeptide comprises a glycine-serine linker.

6. The chimeric polypeptide of any one of claims 4 to 5, wherein the linking polypeptide has the amino acid sequence (GGS)n wherein n is an integer from 1 to 50.

7. The chimeric polypeptide of claim 6, wherein n is 18, 15, 12, 9, 6, or 3.

8. The chimeric polypeptide of claim 7, wherein n is 3.

9. The polypeptide of any one of claims 4 to 8, wherein the linking polypeptide comprises an amino acid sequence at least about 80%, 85%, 90%, 95%, 96%, 97%, 98%, 99%, or 100% sequence identity to any one of SEQ ID NOS: 25-28.

10. The chimeric polypeptide of any one of claims 1 to 2, wherein the linking polypeptide comprises a hinge domain capable of promoting oligomer formation of the chimeric polypeptide via intermolecular disulfide bonding.

11. The chimeric polypeptide of claim 10, wherein the hinge domain is derived from a CD8α hinge domain, a CD28 hinge domain, a PD-1 hinge domain, a CTLA4 hinge domain, an OX40 hinge domain, an IgG1 hinge domain, an IgG2 hinge domain, an IgG3 hinge domain, and an IgG4 hinge domain, or a functional variant of any thereof.

12. The chimeric polypeptide of any one of claims 10 to 11, wherein the hinge domain is derived from a CD8α hinge domain or a functional variant thereof.

13. The chimeric polypeptide of any one of claims 10 to 11, wherein the hinge domain is derived from a CD28 hinge domain or a functional variant thereof.

14. The chimeric polypeptide of any one of claims 10 to 11, wherein the hinge domain is derived from an OX40 hinge domain or a functional variant thereof.

15. The chimeric polypeptide of any one of claims 10 to 11, wherein the hinge domain is derived from an IgG4 hinge domain or a functional variant thereof.

16. The chimeric polypeptide of any one of claims 10 to 15, wherein the hinge domain comprises an amino acid sequence having at least about 80%, 85%, 90%, 95%, 96%, 97%, 98%, 99%, or 100% sequence identity to any one of SEQ ID NOS: 20-24.

17. The chimeric polypeptide of any one of claims 2 to 16, wherein the stop-transfer-sequence comprises an amino acid sequence having at least about 80%, 85%, 90%, 95%, 96%, 97%, 98%, 99%, or 100% sequence identity to any one of SEQ ID NOS: 39-54.

18. The chimeric polypeptide of any one of claims 1 to 17, wherein the transmembrane domain comprises an amino acid sequence having at least 80%, 85%, 90%, 95%, 96%, 97%, 98%, 99%, or 100% sequence identity to any one of SEQ ID NOS: 29-38.

19. The chimeric polypeptide of any one of claims 1 to 18, wherein the extracellular domain comprises an antigen-binding moiety capable of binding to a ligand on the surface of a cell.

20. The chimeric polypeptide of any one of claims 1 to 19, wherein the cell is a pathogenic cell.

21. The chimeric polypeptide of any one of claims 1 to 20, wherein the ligand comprises a protein or a carbohydrate.

22. The chimeric polypeptide of any one of claims 1 to 21, wherein the ligand is selected from the group consisting of CD1, CD1a, CD1b, CD1c, CD1d, CD1e, CD2, CD3d, CD3e, CD3g, CD4, CD5, CD7, CD8a, CD8b, CD19, CD20, CD21, CD22, CD23, CD25, CD27, CD28, CD33, CD34, CD40, CD45, CD48, CD52, CD59, CD66, CD70, CD71, CD72, CD73, CD79A, CD79B, CD80 (B7.1), CD86 (B7.2), CD94, CD95, CD134, CD140 (PDGFR4), CD152, CD154, CD158, CD178, CD181 (CXCR1), CD182 (CXCR2), CD183 (CXCR3), CD210, CD246, CD252, CD253, CD261, CD262, CD273 (PD-L2), CD274 (PD-L1), CD276 (B7H3), CD279, CD295, CD339 (JAG1), CD340 (HER2), EGFR, FGFR2, CEA, AFP, CA125, MUC-1, MAGE, alkaline phosphatase, placental-like 2 (ALPPL2), B-cell maturation antigen (BCMA), green fluorescent protein (GFP), enhanced green fluorescent protein (EGFP), and signal regulatory protein α (SIRPα).

23. The chimeric polypeptide of any one of claims 1 to 22, wherein the ligand is selected from cell surface receptors, adhesion proteins, integrins, mucins, lectins, tumor-associated antigens, and tumor-specific antigens.

24. The chimeric polypeptide of any one of claims 1 to 23, wherein the ligand is a tumor-associated antigen or a tumor-specific associated antigen.

25. The chimeric polypeptide of any one of claims 1 to 24, wherein the extracellular binding domain comprises the ligand-binding portion of a receptor.

26. The chimeric polypeptide of any one of claims 19 to 25, wherein the antigen-binding moiety is selected from the group consisting of an antibody, a nanobody, a diabody, a triabody, or a minibody, a F(ab′)₂ fragment, a Fab fragment, a single chain variable fragment (scFv), a single domain antibody (sdAb), or a functional fragment thereof.

27. The chimeric polypeptide of claim 26, wherein the antigen-binding moiety comprises an scFv.

28. The chimeric polypeptide of any one of claims 19 to 27, wherein the antigen-binding moiety is a tumor-associated antigen selected from the group consisting of CD19, B7H3 (CD276), BCMA (CD269), ALPPL2, CD123, CD171, CD179a, CD20, CD213A2, CD22, CD24, CD246, CD272, CD30, CD33, CD38, CD44v6, CD46, CD71, CD97, CEA, CLDN6, CLECL1, CS-1, EGFR, EGFRvIII, ELF2M, EpCAM, EphA2, Ephrin B2, FAP, FLT3, GD2, GD3, GM3, GPRC5D, HER2 (ERBB2/neu), IGLL1, IL-11Rα, KIT (CD117), MUC1, NCAM, PAP, PDGFR-β, PRSS21, PSCA, PSMA, ROR1, SIRPα, SSEA-4, TAG72, TEM1/CD248, TEM7R, TSHR, VEGFR2, ALPI, citrullinated vimentin, cMet, and Axl.

29. The chimeric polypeptide of claim 28, wherein the tumor-associated antigen is CD19, CEA, HER2, MUC1, CD20, or EGFR.

30. The chimeric polypeptide of claim 29, wherein the tumor-associated antigen is CD19.

31. The chimeric polypeptide of any one of claims 1 to 30, wherein the ligand-inducible proteolytic cleavage site is a γ-secretase cleavage site.

32. The chimeric polypeptide of any one of claims 1 to 31, wherein the ZTE comprises:
(a) a first domain comprising a DNA-binding zinc finger protein domain (ZF protein domain), and
(b) a second domain through which the ZTE exerts its effect (effector domain), wherein the ZTE having the Formula I:
[effector domain]ₐ-[ZF protein domain]-[effector domain]_{b} (Formula I),
wherein a and b are each independently an integer from 0 to 5, and at least one of a and b is not 0;
wherein the ZF protein domain comprises 1 to about 10 zinc finger arrays (ZFA);
wherein the ZFA comprises about 6 to about 8 zinc finger motifs having the Formula II (from N-terminal to C-terminal):
X_{c}CX_{d}CXₑ-(helix)-HX_{f}H-L² (Formula II),
wherein L² is a linker peptide having about 4-6 amino acid residues, C is Cys, H is His, each X is independently any amino acid, c is an integer from 0 to 3, d is an integer from 1 to 5, e is an integer from 2 to 7, f is an integer from 3 to 6, and (helix) is a peptide domain of about 6 amino acids that forms an α-helix,
wherein the ZFA is capable of binding a specific nucleic acid sequence.

33. The chimeric polypeptide of claim 32, wherein the ZFA of the ZTE is capable of specifically binding to a target nucleic acid sequence selected from the group consisting of SEQ ID NOs: 61-71.

34. The chimeric polypeptide of claim 32, wherein the ZFA comprises a sequence having at least about 90% identity to a sequence selected from the group consisting of SEQ ID NOs: 55-60.

35. The chimeric polypeptide of claim 32, wherein the ZFA has a sequence having about 100% identity to a sequence selected from the group consisting of SEQ ID NOs: 55-60.

36. The chimeric polypeptide of any one of claims 32 to 35, wherein the effector domain comprises an effector domain selected from the group consisting of a transcription activating domain, a transcription repressor domain, or an epigenetic effector domain.

37. The chimeric polypeptide of claim 36, wherein the effector domain comprises a transcription activating domain selected from the group consisting of Herpes Simplex Virus Protein 16 (HSV VP16) activation domain; an activation domain consisting of four tandem copies of VP16 (VP64); a p65 activation domain of NFκB; an Epstein-Barr virus R transactivator activation domain (Rta); a tripartite activator consisting of VP64, and Rta activation domains (VPR); and a histone acetyltransferase core domain of the human E1A-associated protein p300 (p300 HAT core activation domain).

38. The chimeric polypeptide of claim 36, wherein the effector domain comprises a transcription repressor domain selected from the group consisting of a Kruppel associated box repression domain (KRAB); a Repressor Element Silencing Transcription Factor repression domain (REST); a WRPW motif of the hairy-related basic helix-loop-helix repressor proteins repression domain (WRPW); a DNA (cytosine-5)-methyltransferase 3B repression domain (DNMT3B); and an HP1 alpha chromoshadow repression domain.

39. The chimeric polypeptide of claim 36, wherein the effector domain comprises an epigenetic effector domain selected from the group consisting of a DNA methyltransferase DNMT (DNMT1, DNMT3), HAT1, GCN5, PCAF, MLL, SET, DOT1, SUV39H, G9a, KAT2A/B, EZH1/2, TET1/2, a SIRT family protein effector domain, a histone deacetylase, LSD1, and a KDM family protein effector domain.

40. The chimeric polypeptide of any one of claims 36 to 39, wherein the effector domain comprises a domain from a human protein.

41. The chimeric polypeptide of any one of claims 1 to 40, wherein the intracellular domain further comprises a nuclear transport signal sequence.

42. The chimeric polypeptide of any one of claims 1 to 41, wherein the chimeric polypeptide comprises an amino acid sequence having at least 80% sequence identity to any one of SEQ ID NOS: 1-9 and 113-123.

43. A recombinant nucleic acid comprising a nucleotide sequence that encodes a chimeric polypeptide according to any one of claims 1 to 42.

44. The recombinant nucleic acid of claim 43, wherein the nucleotide sequence is incorporated into an expression cassette or an expression vector.

45. The recombinant nucleic acid of claim 44, wherein the expression vector is a viral vector.

46. The recombinant nucleic acid of claim 45, wherein the viral vector is a lentiviral vector, an adenovirus vector, an adeno-associated virus vector, or a retroviral vector.

47. The recombinant nucleic acid of any one of claims 43 to 46, wherein the recombinant nucleic acid further comprises a response element, wherein the response element comprises:
a. a ZFA target sequence;
b. an engineered responsive promoter operably linked to the ZF target sequence; and
c. a polynucleotide of interest.

48. The recombinant nucleic acid of claim 47, wherein the polynucleotide of interest encodes a regulatory RNA, a regulatory protein, a therapeutic protein, or a detectable label.

49. The recombinant nucleic acid of claim 48, wherein the detectable label is a fluorescent protein.

50. The recombinant nucleic acid of claim 48, wherein the therapeutic protein is a chimeric antigen receptor (CAR).

51. The recombinant nucleic acid of claim 48, wherein the regulatory RNA is an siRNA, shRNA, or miRNA.

52. A recombinant cell comprising:
a) a chimeric polypeptide according to any one of claims 1 to 42; and/or
b) a recombinant nucleic acid according to any one of claims 43 to 51.

53. The recombinant cell of claim 52, wherein the recombinant cell is a eukaryotic cell.

54. The recombinant cell of claim 53, wherein the eukaryotic cell is a mammalian cell.

55. The recombinant cell of claim 54, wherein the mammalian cell is an immune cell, a neuron, an epithelial cell, and endothelial cell, or a stem cell.

56. The recombinant cell of any one of claims 52 to 55, further comprising an engineered response element comprising i) a ZFA target sequence to which a ZFA of the ZTE of the chimeric polypeptide specifically binds, ii) a promoter sequence, wherein the nucleic acid target sequence is operably linked to the 5′ end of the promoter sequence, and iii) a polynucleotide of interest operably linked to the promoter sequence, wherein binding of the ZTE to the ZFA target sequence modulates transcription initiation of a polynucleotide of interest.

57. The recombinant cell of claim 56, wherein the engineered response element is present in a nucleic acid vector, plasmid, DNA minicircle, minichromosome, or host chromosome.

58. The recombinant cell of claim 56 or 57, wherein the polynucleotide of interest encodes a protein, regulatory RNA, or an antisense oligonucleotide.

59. The recombinant cell of any one of claims 56 to 58, wherein the ZFA target sequence comprises a sequence that is orthogonal to the recombinant cell genome.

60. The recombinant cell of any one of claims 56 to 59, wherein the ZFA target sequence comprises a nucleotide sequence selected from the group consisting of SEQ ID NOs: 61-71.

61. The recombinant cell of any one of claims 55 to 60, wherein the immune cell is a B cell, a monocyte, a natural killer cell, a basophil, an eosinophil, a neutrophil, a dendritic cell, a macrophage, a regulatory T cell, a helper T cell, a cytotoxic T cell, or other T cell.

62. A cell culture comprising a recombinant cell according to any one of claims 52 to 61, and a culture medium.

63. A method for making an engineered cell, comprising:
a. providing a cell capable of protein expression; and
b. transducing the cell with the recombinant nucleic acid of any one of claims 43 to 51.

64. The method of claim 63, wherein the recombinant nucleic acid is a recombinant nucleic acid of any one of claims 43 to 51, and wherein the method further comprises:
c. transducing the cell with a recombinant nucleic acid that encodes a response element, wherein the response element comprises:
i. a ZFA target sequence;
ii. an engineered responsive promoter operably linked to the ZF target sequence; and
iii. a polynucleotide of interest.

65. The method of claim 63, wherein the recombinant nucleic acid is a recombinant nucleic acid of any one of claims 47 to 51.

66. A pharmaceutical composition comprising:
a) a recombinant nucleic acid according to any one of claims 43 to 51; and/or
b) a recombinant cell according to any one of claims 52 to 61; and
c) a pharmaceutically acceptable carrier.

67. The pharmaceutical composition of claim 66, wherein the composition comprises a recombinant nucleic acid according to any one of claims 43 to 51, and a pharmaceutically acceptable carrier.

68. The pharmaceutical composition of claim 67, wherein the recombinant nucleic acid is encapsulated in a viral capsid or a lipid nanoparticle.

69. A method for modulating an activity of a target cell in an individual, the method comprising administering to the individual an effective number of the recombinant cells according to any one of claims 52 to 61, wherein the recombinant cells modulate an activity of the target cell in the individual.

70. The method of claim 69, wherein the wherein the target cell is a pathogenic cell.

71. The method of claim 71, wherein the wherein the target cell is a cancer cell.

72. The method of any one of claims 69 to 71, wherein modulation of the activity the target cell results in the death of the target cell.

73. A method for modulating an activity of a cell, the method comprising:
a) providing a recombinant cell according to any one of claims 52 to 61; and
b) contacting the recombinant cell with the selected ligand, wherein binding of the selected ligand to the extracellular ligand-binding domain results in cleavage of a ligand-inducible proteolytic cleavage site and release of the intracellular domain, wherein the release of the intracellular domain results in modulation of an activity of the recombinant cell.

74. The method of claim 73, wherein the contacting is carried out in vivo, ex vivo, or in vitro.

75. The method of claim 73 or 74, wherein the release of the intracellular domain results in binding of the ZTE of the released intracellular domain to a ZFA target sequence, which results in modulation of the expression initiation of a polynucleotide of interest, which results in modulation of an activity of the recombinant cell.

76. The method of any one of claims 73 to 75, wherein the activity of the cell to be modulated is selected from the group consisting of: expression of a selected gene, proliferation, apoptosis, non-apoptotic death, differentiation, dedifferentiation, migration, secretion of a molecule, cellular adhesion, and cytolytic activity.

77. The method of any one of claims 73 to 76, wherein the ZTE modulates expression of a gene.

78. The method of any one of claims 73 to 76, wherein the ZTE modulates expression of a heterologous gene product.

79. The method of claim 77 or 78, wherein the gene product is selected from the group consisting of chemokine, a chemokine receptor, a chimeric antigen receptor, a cytokine, a cytokine receptor, a differentiation factor, a growth factor, a growth factor receptor, a hormone, a metabolic enzyme, a pathogen-derived protein, a proliferation inducer, a receptor, an RNA guided nuclease, a site-specific nuclease, a T cell receptor, a toxin, a toxin derived protein, a transcriptional regulator, a transcriptional activator, a transcriptional repressor, a translational regulator, a translational activator, a translational repressor, an activating immuno-receptor, an antibody, an apoptosis inhibitor, an apoptosis inducer, an engineered T cell receptor, an immuno-activator, an immuno-inhibitor, an immune cell receptor, and an inhibiting immuno-receptor.

80. The method of any one of claims 73 to 79, wherein the released ZTE modulates differentiation of the cell, and wherein the cell is an immune cell, a stem cell, a progenitor cell, or a precursor cell.

81. A method for the treatment of a health condition in an individual in need thereof, the method comprising administering to the individual a first therapy comprising an effective number of the recombinant cell according to any one of claims 52 to 61, wherein the recombinant cell treats the health condition in the individual.

82. A kit for modulating an activity of a cell, inhibiting a target cell, or treating a health condition in an individual in need thereof, the system comprising:
a) a chimeric polypeptide according to any one of claims 1 to 42;
b) a recombinant nucleic acid according to any one of claims 43 to 51;
c) a recombinant cell according to any one of claims 52 to 61; and/or
d) a pharmaceutical composition according to any one of claims 66 to 68.

83. A kit for modulating an activity of a cell, the kit comprising:
a) a chimeric polypeptide according to any one of claims 1 to 42;
b) a recombinant nucleic acid according to any one of claims 43 to 51; and/or
c) an engineered response element comprising:
i) a ZFA target sequence;
ii) an engineered responsive promoter operably linked to the ZFA target sequence; and
iii) a polynucleotide of interest;
wherein binding of the ZTE to the nucleic acid target sequence modulates transcription initiation of the polynucleotide of interest.

84. The use of a pharmaceutical composition according to any one of claims 66 to 68 for the treatment of a health condition.
